(19) United States
(12) Patent Application Publication
(10) Pub. No.: US 2003/0166026 A1
(43) Pub. Date: Sep. 4, 2003

(54) Identification of specific biomarkers for breast cancer cells

(75) Inventors: Laurie J. Goodman, El Granada, CA (US); Benjamin A. Bowen, Berkeley, CA (US)

Correspondence Address: QUINE INTELLECTUAL PROPERTY LAW GROUP, P.C., P O BOX 458, ALAMEDA, CA, 94501, US

(73) Assignee: Lynx Therapeutics, Inc., Hayward, CA (US)

(21) Appl. No.: 10/339,782

(22) Filed: Jan. 8, 2003

Related U.S. Application Data
(60) Provisional application No. 60/348,053, filed on Jan. 9, 2002.

(51) Int. Cl. C12Q 1/68; G01N 33/574; C07H 21/04; C12N 9/00; C12P 21/02; C12N 5/06

(52) U.S. Cl. 435/7.23; 435/69.3; 435/183; 435/320.1; 435/325; 435/6; 536/23.2

(57) ABSTRACT

Nucleic acids, proteins, antibodies, marker sets and arrays are provided for biomarkers for breast cancer. Methods for detecting breast cancer, modulating breast cancer phenotypes in cells, and for treating a subject with breast cancer are also provided.

### CROSS-REFERENCE TO RELATED APPLICATIONS

This application claims the benefit of U.S. Provisional Application No. 60/348,053 filed Jan. 9, 2002, entitled “IDENTIFICATION OF SPECIFIC BIOMARKERS FOR BREAST CANCER CELLS” and naming Laurie Goodman et al. as the inventors. This prior application is hereby incorporated by reference in its entirety.

### STATEMENT AS TO RIGHTS TO INVENTIONS MADE UNDER FEDERALLY SPONSORED RESEARCH AND DEVELOPMENT

Not Applicable.

### FIELD OF THE INVENTION

This invention relates to biomarkers that are relevant to breast cancer, e.g., for identifying breast cancer cells and distinguishing types and/or stages of the breast cancer. The present invention provides nucleotide sequences, polypeptides encoded by these sequences, related probes, marker sets, methods for detecting and monitoring subjects for breast cancer, treatment for breast cancer, and cellular and transgenic models relevant to breast cancer.

### BACKGROUND OF THE INVENTION

Breast cancer is one of the leading causes of death among women worldwide. Human breast tumors are diverse in their development and in their response to treatment. Although various factors have been implicated in causing breast cancer, such as proto-oncogenes, suppressor genes, certain polymorphisms, hereditary factors and hormonal imbalances, the exact causes of breast cancer are still unknown. Therefore, a better understanding of the molecular events that cause breast cancer can lead to early detection and more effective treatment.

Two factors that have been implicated in breast cancer are estrogen and progesterone. Estrogen and progesterone, which are ovarian hormones, have been shown to be responsible for the proliferation of normal and malignant mammary glands. The estrogen receptor (ER) is another molecule that has been implicated as playing a role in breast cancer. At the outset of most breast cancers, the cells are estrogen receptor positive (ER+) and progesterone receptor positive (PgR+). Many of these cells are responsive to hormonal treatment, however there is a subset (about 30-45%) of ER positive breast cancers that do not respond to hormone therapy.

Breast cancer cells that are estrogen receptor negative (ER−) and/or progesterone receptor negative (PgR−) are typically resistant to hormone treatment and often present a more aggressive metastatic phenotype. See, e.g., Sheikh et al., (1994-1995), Why are Estrogen-Receptor-Negative Breast Cancers More Aggressive than the Estrogen-Receptor-Positive Breast Cancers?, Invasion Metastasis 14:329-336. Furthermore, breast cancer cells that were once responsive to hormonal therapy, e.g., estrogen receptor positive (ER+), can become hormone independent by loss the of functional estrogen receptors, which results in the more aggressive metastatic phenotype.

In general, an estrogen receptor positive breast cancer cell is an indicator of differentiation of the cancer and a predictor of disease-free survival between node-negative and node-positive breast cancer. ER− breast cancers are generally more aggressive and metastatic than the ER+ tumors. Metastasis consists of a multiple number of steps between the tumor and the host. When a tumor metastasizes, there is destruction of host barriers and invasion of the tumor into surrounding tissues. Invasion and metastasis are responsible for most cancer-related deaths. See, Sheikh, supra.

Therefore, it would be advantageous to be able to screen and to distinguish those breast cancers that are ER+ from ER− as a way to predict responsiveness to hormonal treatment and a favorable clinical outcome. The present invention provides compositions, methods and kits for distinguishing breast cancer cells from normal mammary epithelium. In addition, compositions, methods and kits are also provided for distinguishing an ER+ from an ER− breast cancer cell. Other features that will become apparent upon review of the accompanying disclosure are also provided.

### SUMMARY OF THE INVENTION

The present invention relates to a set of polynucleotide sequences associated with breast cancer, exemplified by SEQ ID NO: 1 through SEQ ID NO: 491 and polypeptide sequences associated with breast cancer, exemplified by SEQ ID NO: 492. In a first aspect, the invention relates to compositions including one or more nucleic acid expression vectors including the polynucleotides sequences of the invention. For example, such expression vectors include nucleic acids including at least one polynucleotide sequence selected from SEQ ID NOs: 1-491. Similarly, sequences that hybridize under stringent hybridization conditions, or that are at least about 70%, (or at least about 75%, about 80%, about 85%, about 90%, about 95%, about 97%, about 98%, or at least about 99%) identical to one or more of SEQ ID NO: 1-491 can be included in the expression vectors of the invention. In addition, expression vectors, including polynucleotide sequences that encode a polypeptide sequence, e.g., SEQ ID NO: 492 or encoded by a polynucleotide sequence selected from SEQ ID NO: 1-491, or conservative variations thereof, are compositions of the invention. Likewise, expression vectors incorporating nucleic acids with subsequences of at least about 10 contiguous nucleotides of, e.g., SEQ ID NOs: 1-491 (or at least about 12, about 14, about 16, or about 17 or more contiguous nucleotides of one of the designated sequences) are included among the compositions of the invention. Polynucleotide sequences that correspond to sequences that are physically linked in the human genome to a nucleic acid comprising one or more of the above polynucleotide sequences are also polynucleotides of the invention. The polynucleotide sequences of the invention also include polynucleotide sequences complementary to any one of the above polynucleotide sequences described above. In some embodiments, the expression vector includes a promoter operably linked to one or more of the nucleic acids described above. Such expression vectors can encode expression products such as sense or antisense RNAs, or polypeptides.

Polypeptides having an amino acid sequence, e.g., of SEQ ID NO: 492, and conservative variants thereof, are also a feature of the invention, as are polypeptides encoded by a polynucleotide sequence of the invention (e.g., SEQ ID NO: 1-SEQ ID NO: 491, sequences that hybridize under stringent conditions to any one of SEQ ID NO: 1-SEQ ID NO: 491, sequences that are at least about 70% identical to any one of SEQ ID NO: 1-SEQ ID NO: 491, sequences that encode a polypeptide or conservative variations thereof encoded by any one of SEQ ID NO: 1-SEQ ID NO: 491, sequences complementary to any such sequences, or subsequences thereof). Polypeptides (and oligopeptides and peptides) including amino acid subsequences of, e.g., SEQ ID NO: 492, are also a feature of the invention. For example, fusion proteins including a polypeptide of, e.g., SEQ ID NO: 492 or encoded by any one of SEQ ID NO: 1 through SEQ ID NO: 491, or a subsequence, e.g., an antigenic subsequence thereof, are included in the polypeptides of the invention. Likewise, proteins having a sequence, e.g., of SEQ ID NO: 492 or encoded by any one of SEQ ID NO: 1 through SEQ ID NO: 491, and homologous or variant polypeptides and a peptide or polypeptide tag, such as a reporter peptide or polypeptide, localization signal or sequence, or antigenic epitope, are included among the polypeptides of the invention. An array of polypeptides comprising two or more different isolated or recombinant polypeptides, described above, is also a feature of the present invention.

Cells, including an expression vector, and/or expressing a polypeptide as described above, are also a feature of the invention. In certain embodiments, the expressed polypeptide is encoded by an exogenous polynucleotide, e.g., an expression vector. Such expression vectors typically include a polynucleotide sequence encoding the polypeptide of interest, operably linked to, and under the transcriptional regulation of, a constitutive or inducible promoter. In other embodiments, the polypeptide is encoded by an endogenous polynucleotide sequence activated by an exogenous promoter and/or enhancer.

Antibodies specific for isolated or recombinant polypeptides or peptides of the invention, e.g., polypeptide having a sequence or subsequence of SEQ ID NO: 492 and/or encoded by SEQ ID NO: 1-SEQ ID NO: 491 (or a subsequence thereof) or a sequence complementary thereto, and conservatively modified variants, etc., are also a feature of the invention. Such specific antibodies can be either derived from a polyclonal antiserum or can be monoclonal antibodies. For example, such antibodies are specific for an epitope including or derived from a sequence or subsequence of SEQ ID NO: 492 and/or encoded by one of SEQ ID NO: 1-SEQ ID NO: 491. One or more isolated or recombinant polypeptides that bind to the antibodies of the present Invention are also included.

Compositions comprising any of the above nucleic acid, isolated or recombinant polypeptides, peptides, antibodies or cells optionally include an excipient to facilitate admninistration, e.g., a pharmaceutically acceptable excipient. Transgenic animals, which include the compositions described above, are also a feature of the invention. In one embodiment of the invention, methods include treating breast cancer by administering to a patient an effective amount of at least one expression vector and/or an effective amount of at least one isolated or recombinant polypeptide described above are also provided in the present invention.

Another aspect of the invention provides labeled nucleic acid or polypeptide probes. For example, nucleic acid probes of the invention include DNA or RNA molecules incorporating a polynucleotide sequence of the invention e.g., selected from SEQ ID NO: 1-SEQ ID NO: 491, sequences that hybridize under stringent conditions to any one of SEQ ID NO: 1-SEQ ID NO: 491, sequences that are at least about 70% identical to any one of SEQ ID NO: 1-SEQ ID NO: 491, sequences that encode a polypeptide or peptide comprising a subsequence encoded by any one of SEQ ID NO: 1-SEQ ID NO: 491, sequences that are physically linked in the human genome to any one of SEQ ID NO: 1-SEQ ID NO: 491, sequences complementary to any such sequences, or subsequences thereof including at least about 10 contiguous nucleotides. Optionally, the subsequences include at least about 12 contiguous nucleotides of one of, e.g., SEQ ID NOs: 1-491. Often such subsequences include at least about 14 contiguous nucleotides, typically at least 16 contiguous nucleotides, and usually at least about 17 or more contiguous nucleotides, e.g., of SEQ ID NO: 1 to SEQ ID NO: 491. These nucleic acid probes can be, e.g., synthetic oligonucleotides and probes, cDNA molecules, amplification products (e.g., produced by PCR or LCR), transcripts, or restriction fragments.

In other embodiments, the labeled probes are polypeptides, such as, polypeptides or peptides with an amino acid sequence or subsequence (e.g., peptide subsequence comprising at least 6 amino acids) of SEQ ID NO: 492, or encoded by a polynucleotide of the invention, e.g., selected from SEQ ID NO: 1 through SEQ ID NO: 491, including peptide subsequences. Antibodies specific for such polypeptides or peptides are also a feature of the invention (as are polypeptides which bind to such antibodies). For example, a polypeptide probe can be a fusion protein, or a polypeptide with an epitope tag. A peptide probe can be an antigenic peptide derived from, e.g., SEQ ID NO: 492 or encoded by one of SEQ ID NO: 1 through SEQ ID NO: 491.

The label of the nucleic acid, polypeptide or antibody probe can be any of a variety of detectable moieties including isotopic, fluorescent, fluorogenic, or colorimetric labels.

The labeled probe can include an array of probes comprising a plurality of nucleic acids. The plurality of nucleic acids include two or more polynucleotides of the invention, e.g., selected from SEQ ID NO: 1-SEQ ID NO: 491, sequences that hybridize under stringent conditions to any one of SEQ ID NO: 1-SEQ ID NO: 491, sequences that are at least about 70% identical to any one of SEQ ID NO: 1-SEQ ID NO: 491, sequences that encode a polypeptide or peptide comprising a subsequence encoded by any one of SEQ ID NO: 1-SEQ ID NO: 491, sequences that are physically linked in the human genome to any one of SEQ ID NO: 1-SEQ ID NO: 491, and sequences complementary to any such sequences, or subsequences thereof. The nucleic acids are optionally logically or physically arrayed.

In another aspect, the invention relates to a marker set, e.g., for predicting breast cancer, e.g., by identifying breast cancer cells and/or distinguishing breast cancer cell types. Marker sets can be used to predict at least one characteristic of a breast cancer cell, e.g., transformation state, invasiveness, progression stage, a specific protein induced or suppressed, a specific protein expressed on or absent from the surface of the breast cell, and the like. Such marker sets can include a plurality of members, where the plurality of members include nucleic acids, polypeptides or peptides and/or antibodies. Marker sets can include two or more of one type of member or optionally can include one or more of two or more different types of members. For example, marker sets can include a plurality of nucleic acids including one or more polynucleotide sequence selected from SEQ ID NO: 1 to SEQ ID NO: 491, or conservative modifications thereof; polynucleotide sequences that hybridize under stringent hybridization conditions, or that are at least about 70%, (or at least about 75%, about 80%, about 85%, about 90%, about 95%, about 97%, about 98%, or at least about 99%) identical to one or more of SEQ ID NOs: 1-491; sequences complementary to any such sequences or subsequences thereof including at least about 10 contiguous nucleotides, e.g., of SEQ ID NOs: 1-491 (or at least about 12, about 14, about 16, or about 17 or more contiguous nucleotides of one of the designated sequences). For example, a marker set can be used for predicting a type of breast cancer, e.g., an ER+ breast cancer from an ER− breast cancer, or e.g., such as a subset of breast cancer, or e.g., such as a cancer type that is indicative of a favorable or unfavorable outcome, using at least one polynucleotide of invention described above, e.g., selected from SEQ ID NO: 1-SEQ ID NO: 286.

In one embodiment, the marker set includes a plurality of oligonucleotides, such as synthetic oligonucleotides. In other embodiments, the marker set includes expression products, amplification products, nucleic acid probes, labeled nucleic acid probes or the like. The marker set of the invention can also include multiple nucleic acids selected from among different molecular classifications, e.g., oligonucleotides, expression products (such as cDNAs), amplification products, restriction fragments, etc. In one embodiment, the marker set is made up of nucleic acids including polynucleotide sequences corresponding to each of SEQ ID NO: 1 through SEQ ID NO: 491. In another embodiment, each member of the marker set comprises at least about 10 contiguous nucleotides, e.g., selected from SEQ ID NO: 1-SEQ ID NO: 491. In other aspects, the plurality of members of the marker set together comprise a plurality of sequences or subsequences selected from a plurality of nucleic acids represented by the polynucleotides of the invention. In another embodiment, the marker set includes a plurality of members, where a majority of members of the marker set together comprise a majority of subsequences from a majority of the polynucleotides of the invention.

Markers of the invention can also be polypeptides, e.g., polypeptide of SEQ ID NO: 492 and/or polypeptides encoded by polynucleotides of the invention, e.g., selected from SEQ ID NO: 1-SEQ ID NO: 491, or polypeptide or peptide subsequences thereof. For example, a peptide subsequence of, e.g., SEQ ID NO: 492, comprises at least about 10 contiguous amino acids, often at least about 15 contiguous amino acids, frequently at least about 20 contiguous amino acids. Marker sets can include one or more polypeptides or peptides comprising an amino acid, e.g., SEQ ID NO: 492 and/or encoded by a polynucleotide sequence of the invention, e.g., sequence selected from SEQ ID NO: 1 to SEQ ID NO: 491. Typically, the marker set can include a plurality of polypeptides or peptides.

Markers of the invention can also be antibodies. Marker sets can include one or more antibodies specific for a polypeptide or peptide or subsequence of a polypeptide of the invention, e.g., SEQ ID NO: 492 and/or encoded by a polynucleotide of the invention, e.g., a sequence selected from SEQ ID NO: 1 to SEQ ID NO: 491, e.g., monoclonal or polyclonal antibodies or antisera specific for an epitope derived an amino acid, e.g., SEQ ID NO: 492 and/or encoded by a sequence of a polynucleotide of the invention, e.g., selected from one of SEQ ID NO: 1 through SEQ ID NO: 491. Optionally, the marker set can include a plurality of antibodies.

In certain embodiments, the marker set is logically or physically arrayed. For example, the members of the marker set, whether nucleic acid, polypeptide, peptide, antibody, or a combination thereof, can be physically arrayed in a solid phase or liquid phase array, such as a bead (or microbead) array. Arrays including a plurality of SEQ ID NO: 1 to SEQ ID NO: 492, or antibodies specific therefor, are also a feature of the invention. In some embodiments, the arrays include members corresponding to majority of SEQ ID NO: 1 to SEQ ID NO: 492, or antibodies specific therefor. In one embodiment, the array includes members corresponding to each of SEQ ID NO: 1 to SEQ ID NO: 492, or antibodies specific therefor. In an embodiment, the marker set is comprised of at least about 10 contiguous nucleotides of each of SEQ ID NO: 1-SEQ ID NO: 491, at least about 10 contiguous nucleotides of a plurality of SEQ ID NO: 1-SEQ ID NO: 491, at least about 10 contiguous nucleotides of a majority of SEQ ID NO: 1-SEQ ID NO: 491, or complimentary sequences thereof. In an embodiment, the marker set is a mixed marker set including members that are selected from nucleic acids, polypeptides or peptides, and antibodies.

In one embodiment, the marker set of the invention is used to predict breast cancer, e.g., identifying breast cancer cells and/or distinguishing breast cancer cell types by hybridizing one or more nucleic acids of the marker set to a DNA or RNA sample from a cell or tissue (e.g., a patient, e.g., a tissue array, e.g., cDNA microarray), and detecting at least one polymorphic polynucleotide or differentially expressed expression product in the sample. In another related embodiment, differentially expressed expression products are detected using an array, e.g., an antibody array.

Another aspect of the invention provides methods for modulating at least one characteristic of a breast cell, e.g., the transformation and/or the progression of a breast cell, in a cell, tissue or organism, such as a cell line or tissue of a human mammal or a non-human mammal, e.g., a mouse, a rat, a rabbit, a dog, a pig, a sheep or a non-human primate. Such characteristics include, e.g., transformation state, invasiveness, progression stage, a specific protein induced or suppressed, a specific protein expressed on or absent from the surface of the breast cell, and the like. For example, the methods of the invention for modulating at least one characteristic of a breast cell optionally include modulating, expression or activity of at least one polypeptide of the invention, e.g., SEQ ID NO: 492, or at least one polypeptide encoded by a polynucleotide of the invention. The above mentioned polypeptides can be involved in, e.g., the transformation of normal mammary epithelium to breast cancer cells, e.g., which can be either ER+or ER−, or e.g., which can be ductal carcinoma in situ, or e.g., which can be a non-infiltating cancer, and/or involved in, e.g., the regulation of the critical transition in the progression of malignant breast cancer marked by a type of breast cancer cells and/or the transition of a breast cancer cell, e.g., ER+ breast cancer cells to ER− breast cancer cells, e.g., polypeptide sequence or subsequence encoded by SEQ ID Nos: 1-286.

In one embodiment, methods for modulating at least one characteristic of a breast cancer cell, e.g., the transformation of breast cells, e.g., from normal mammary epithelial cells to breast cancer cells, optionally include modulating expression or activity of at least one polypeptide of the invention, e.g., SEQ ID NO: 492, or at least one polypeptide encoded by a polynucleotide of the invention, such as a nucleic acid with a polynucleotide sequence selected from SEQ ID NO: 1-SEQ ID NO: 491, sequences that hybridize under stringent conditions to any one of SEQ ID NO: 1-SEQ ID NO: 491, sequences that are at least about 70% identical to any one of SEQ ID NO: 1-SEQ ID NO: 491, sequences that encode a polypeptide or peptide comprising a subsequence encoded by any one of SEQ ID NO: 1-SEQ ID NO: 491, sequences that are physically linked in the human genome to any one of SEQ ID NO: 1-SEQ ID NO: 491, sequences complementary to any such sequences, or subsequences thereof including at least about 10 contiguous nucleotide of, e.g., SEQ ID Nos: 1-491 (or at least about 12, about 14, about 16, or about 17 or more contiguous nucleotides of one of the designated sequences). In another embodiment, modulating at least one characteristic of a breast cancer cell, e.g., the type of breast cancer cell, e.g., from ER+ to ER− breast cancer cell, optionally include modulating expression or activity of at least one polypeptide, e.g., SEQ ID NO: 492 and/or at least one polynucleotide of the invention, e.g., selected from SEQ ID NO: 1 to SEQ ID NO: 286.

In one embodiment, breast cell transformation and/or breast cancer progression is modulated by modulating expression or activity of at least one polypeptide contributing to a breast cancer phenotype. In an embodiment, expression is modulated by expressing an exogenous nucleic acid including a polynucleotide of the invention, e.g., a sequence selected from SEQ ID NO: 1 to SEQ ID NO: 491. In other embodiments, expression of an endogenous nucleic acid including a subsequence corresponding to one of the polynucleotide sequences of the invention, e.g., selected from SEQ ID NO: 1 through SEQ ID NO: 491, is induced or suppressed, for example, by introducing and/or integrating an exogenous nucleic acid including at least one promoter that regulates expression of the endogenous nucleic acid. In other embodiments, expression or activity is modulated in response to a carcinogenic signal, a pharmaceutical agent or the like.

In some embodiments, the methods involve detecting altered expression or activity of an expression product, such as an RNA or polypeptide, encoded by a nucleic acid including a polynucleotide sequence selected from SEQ ID NO: 1 to SEQ ID NO: 491. In some cases, altered expression or activity in response to a carcinogenic signal is detected. In other cases, altered expression or activity in response to a pharmaceutical agent is detected. In certain embodiments, a plurality of expression products are detected, e.g., in a high-throughput assay. For example, a plurality of expression products can be detected in an array, such as a bead array, or a tissue array.

In an embodiment, a data record related to the altered expression or activity is recorded in a database. For example, a data record can be a character string recorded in a database made up of a plurality of character strings recorded in a computer or on a computer readable medium.

In one embodiment, the methods involve identifying a breast cancer gene. The methods of the invention for identifying a breast cancer gene involve providing at least one nucleic acid, such as, a polynucleotide sequence, e.g., selected from SEQ ID NO: 1-SEQ ID NO: 491, or a sequence that hybridize under stringent conditions to any one of SEQ ID NO: 1-SEQ ID NO: 491, or a sequence that is at least about 70% identical to any one of SEQ ID NO: 1-SEQ ID NO: 491, or a sequence that encodes a polypeptide or peptide comprising a subsequence encoded by any one of SEQ ID NO: 1-SEQ ID NO: 491, or a sequence that is physically linked in the human genome to any one of SEQ ID NO: 1-SEQ ID NO: 491, or a sequence complementary to any such sequences, or subsequences thereof including at least about 10 contiguous nucleotides of, e.g., SEQ ID NO; 1-491, and identifying at least one nucleic acid corresponding to a breast cancer gene. Optionally, the subsequence comprises at least about 12 contiguous nucleotides of, e.g., SEQ ID NO: 1-SEQ ID NO: 491, or at least about 14 contiguous nucleotides of, e.g., SEQ ID NO: 1-SEQ ID NO: 491, or at least about 15 contiguous nucleotides of, e.g., SEQ ID NO: 1-SEQ ID NO: 491 or at least about 17 contiguous nucleotides of, e.g., SEQ ID NO: 1-SEQ ID NO: 491. The methods can include providing at least one expression vector comprising a polynucleotide sequence of invention. Optionally, the methods include providing at least one probe comprising a polynucleotide sequence of the invention or polypeptide of the invention or antibody thereof; and, hybridizing at least one probe to an expression product of a breast cancer gene. In another embodiment, at least one nucleic acid comprises amplifying a target sequence comprising a polynucleotide sequence of the invention, e.g., selected from SEQ ID NO: 1-SEQ ID NO: 491. For example, the amplification can be accomplished using a quantitative reverse transcriptase-polymerase chain reaction (RT-PCR) method.

Methods for identifying a breast cancer gene can further comprise identifying a target sequence that is differentially expressed in a transformed breast cell compared to a non-transformed breast cell, e.g., when a normal mammary epithelia cell is transformed to a breast cancer cell, or that is differentially expressed in the progression of a breast cancer cell to a different stage, e.g., when a breast cancer cell is an ER+ cell or an ER− cell type. Methods for identifying a breast cancer can also further comprise detecting altered expression or activity of a product encoded by the nucleic acid comprising the polynucleotide sequence, e.g., mRNA. The detection of the altered expression or activity can be accomplished by the analysis of data from a number of techniques, e.g., massively parallel signature sequences (MPSS), differential hybridization screening, subtractive library construction, representative difference analysis (RDA), differential display, conventional cDNA array hybridization, serial analysis of gene expression (SAGE), a combination of suppression subtractive hybridization and cDNA microarrays and the like. Optionally, the altered expression or activity is determined to be differentially expressed to a p<0.05 level of confidence, optionally, to a p<0.01 level of confidence, or optionally, to a p<0.001 level of confidence. Many factors can cause and contribute to an altered expression or activity, such as a drug, e.g., tamoxifen. Other examples of altered expression or activity include, e.g., the transformation of a cell from a normal cell to a cancerous cell, e.g., normal mammary epithelia to breast cancer cell, the progression of breast cancer, e.g., ER+ breast cancer cell to an ER− breast cancer cell, can be caused by a carcinogenic signal.

In another aspect, the invention provides methods for detecting breast cancer in a subject, such as a human subject. The methods of the invention for detecting breast cancer involve providing a subject cell or tissue sample of nucleic acids and detecting at least one polymorphic polynucleotide sequence or expression product corresponding to a polynucleotide sequence of the invention, such as: a polynucleotide selected from SEQ ID NO: 1-SEQ ID NO: 491, sequences that hybridize under stringent conditions to any one of SEQ ID NO: 1-SEQ ID NO: 491, sequences that are at least about 70% identical to any one of SEQ ID NO: 1-SEQ ID NO: 491, sequences that encode a polypeptide or peptide comprising a subsequence encoded by any one of SEQ ID NO: 1-SEQ ID NO: 491, sequences that are physically linked in the human genome to any one of SEQ ID NO: 1-SEQ ID NO: 491, sequences complementary to any such sequences, or subsequences thereof including at least about 10 contiguous nucleotides of SEQ ID NOs: 1-491 (or at least about 12, about 14, about 16, or about 17 or more contiguous nucleotides of one of the designated sequences), wherein the polymorphic nucleic acid or expression or activity of the expression product, e.g., an RNA and/or a protein or polypeptide, is correlatable to breast cancer.

Detection of expression products is performed either qualitatively (presence or absence of one or more product of interest) or quantitatively (by monitoring the level of expression of one or more product of interest). In one embodiment, the polymorphic nucleic acid or expression product corresponds to or is encoded by a breast cancer locus on a human chromosome, e.g., 11, for example, 11q13-q14 region. In an embodiment, the expression product is an RNA expression product, such as differentially expressed RNA. The present invention optionally includes monitoring an expression level of a nucleic acid or polypeptide as noted herein for detection of breast cancer in an individual, such as a human, or in a population, such as a human population.

Kits that incorporate one or more of the nucleic acids, polypeptides, antibodies, or arrays noted above are also a feature of the invention. Such kits can include any of the above noted components and further include, e.g., instructions for use of the components in any of the methods noted herein, packaging materials, containers for holding components, and/or the like.

Digital systems which incorporate one or more representations (e.g., character string, data table, or the like) of one or more of the nucleic acids or polypeptides herein are also a feature of the invention.

### BRIEF DESCRIPTION OF THE DRAWINGS

FIG. 1 illustrates one example of the progression of breast cancer. It starts with normal ductal epithelia cells and moves through different stages, ductal carcinoma in situ (DCIS), Infiltrating, Ductal Carcinoma-ER+, Infiltrating Ductal Carcinoma-ER−, to metastatic Infiltrating ductal carcinoma cells (ER−, Her2+).

FIG. 2 illustrates 59-4 expression along with Glutathione-S-Transferase, a known ER+ marker, in ER+ and ER− cell lines.

FIG. 3 illustrates 59-4 expression in tumors and cells-lines, e.g., characterized by being either the ER+ or ER− phenotype.

FIG. 4 illustrates tissue specific expression of 59-4.

FIG. 5 illustrates the mapping of 59-4 to Chromosome 11q13-q14.

FIG. 6 illustrates 59-4 mapping to approximately MEN1.

FIG. 7 illustrates that Tamoxifen increases the expression of 59-4 in an ER+ cell line that showed increased expression of 59-4.

FIG. 8 shows contrast photographs of three different human breast cancer cell lines, MCF-7, BT-20 and MDA-MB-23 1.

### DETAILED DESCRIPTION

Breast cancer is a disease that effects many women worldwide. It is a complex process that involves many mechanisms and epithelial cells displaying different phenotypes. As mentioned above, estrogen and the estrogen receptor have been implicated as being involved in evolution/progression of breast cancer. The estrogen receptor is a member of the steroid/thyroid hormone nuclear receptor superfamily. The receptor is activated by the ligand binding of estrogens. When the receptor is activated by ligand binding, it becomes a transcription factor and regulates a number of genes. In breast cancer, the activated receptor can regulate a number of genes involved in the progression of breast cancer. At the outset of most breast cancers, the cells are estrogen receptor positive (ER+). Cells with this phenotype are generally responsive to hormonal treatment, however there are a percentage of ER+ cells that do not respond to hormonal treatment.

Unlike most ER+ breast cancers, ER− breast cancers are generally resistant to hormonal treatment and are often more aggressive metastatic cancer. Furthermore, breast cancer cells that were once estrogen receptor positive can become, during the progression of the disease, hormone independent and develop into a more aggressive cancer.

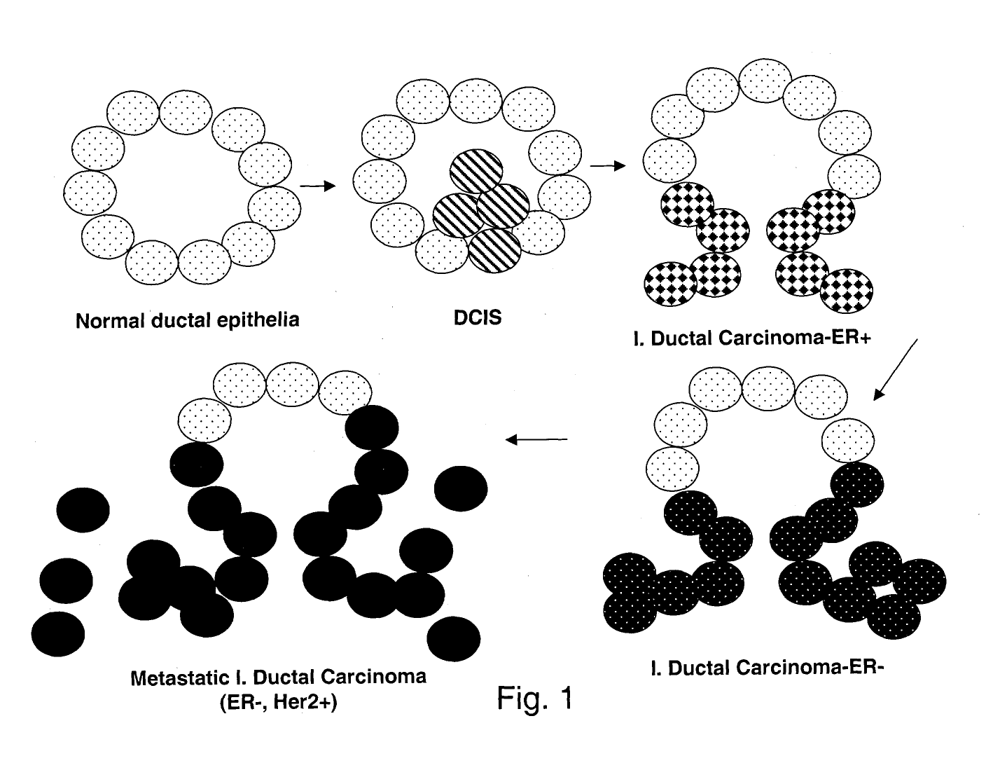

FIG. 1 illustrates one type of progression of breast cancer, e.g., the progression of normal ductal epithelia to metastatic infiltrating ductal carcinoma, which are ER− and Her2+. As seen in FIG. 1, during the progression of the disease, the cells that were once ER+, become ER−. This can be due to the differential expression of genes during the progression of the disease. Differential expression of genes is also seen in normal, verses malignant, human breast tissues, e.g., infiltrating ductal carcinomas, lobular carcinomas, and in situ (non-invasive) ductal carcinomas. See, e.g., Perou et al., (2000), Molecular portraits of human breast tumors, Nature, 406: 747-752.

Some genes have already been identified as being differentially expressed in ER+ breast cancer cell lines compared to ER− breast cancer cell lines, e.g., genes such as cytokeratin 19, GATA-3, CD24, glutathione-S-transferase μ-3, cytokeratin 8, cytokeratin 18, Hsp27, a member of the G protein-coupled receptor superfamily (GPCR-Br), HEK8, neuropeptide Y receptor Y1, p21^{waf-l}, p55^{PIK}, TGFβ1 binding protein, elongation factor 1β2, fructose-1,6, biphosphate, pS2, DEME-2, DEME-31, DEME-47 and DEME-6 are found in ER+ breast cancer cell lines. See Yang et al., (1999), Combining SSH and cDNA microarrays for rapid identification of differentially expressed genes, Nucleic Acids Research 27(6): 1517-1523; Kirschmann et al., (1999) Differentially expressed genes associated with the metastatic phenotype in breast cancer, Breast Cancer Research and Treatment, 55: 127-136; and, Kuang et al., (1998) Differential screening and suppression subtractive hybridization identified genes differentially expressed in an estrogen receptor-positive breast carcinoma cell line, Nucleic Acids Research, 26(4): 1116-1123. Similar expression patterns are also seen in the corresponding tumor from where the cell lines were derived. See, e.g., Wistuba et al., (1998), Comparison of Features of Human Breast Cancer Cell Lines and Their Corresponding Tumors, Clinical Cancer Research, 4: 2931-2938. Other genes can be differentially expressed in ER− cells. For example, high levels of mitogenic growth factors and their receptors are generally present in ER− tumors. There is also generally amplification or altered expression of oncogenes and tumor suppressor genes in ER− tumors. In addition, the expression of moesin, vimentin and the gene called, inversely correlated with estrogen receptor expression (ICERE-1) is associated with an ER− breast cancer phenotype. See, e.g., Carmeci et al., (1998), Moesin expression is associated with the estrogen receptor-negative breast cancer phenotype, Surgery, 124(2): 211-217 and Thompson and Weigel (1998), Characterization of a gene that is inversely correlated with estrogen receptor expression (ICERE-1) in breast carcinomas, Em. J. Biochem., 252: 169-177.

Some genes involved in breast cancer are localized to human chromosome 11. See, e.g., Bekri et al., (1997) Detailed map of a region commonly amplified at 11q12→q14 in human breast carcinoma, Cytogenet. Cell Genet. 79:125-131. Other chromosomes have also been implicated, e.g., human chromosome 17 and 20. The present invention defines an additional sequence, SEQ ID NO: 1, which localizes to a similar region nearby (but probably not similar) on human chromosome 11.

The present invention makes use of tissue culture models of breast cancer cells and types of breast cancer cells to identify expression products that exhibit a significant change in abundance in response to the transformation of a normal mammary epithelial cell to a breast cancer cell, e.g., SEQ ID NO: 1 through SEQ ID NO: 492, and exhibit a significant change in abundance due to the type or progression of a breast cancer cell, e.g., ER+ verses a ER− breast cancer cell, e.g., SEQ ID NO: 1 through SEQ ID NO: 286. These sequences SEQ ID NO: 1 through SEQ ID NO: 492 and sequences complementary thereto are significant as markers and probes for evaluating breast cancer, as well as for identifying the type and/or progression of breast cancer, and for the production of animal and cell culture models useful for the evaluation and monitoring of therapeutic agents and protocols aimed at predicting breast cancer.

### DEFINITIONS

Before describing the present invention in detail, it is to be understood that this invention is not limited to particular compositions, which can, of course vary. It is also to be understood that the terminology used herein is for the purpose of describing particular embodiments only, and is not intended to be limiting. As used in this specification and appended claims, the singular forms “a”, “an”, and “the” include plural referents unless the content and context clearly dictates otherwise. Thus, for example, reference to “an excipient” includes a combination of two or more such excipients, and the like.

Unless defined otherwise, all scientific and technical terms are understood to have the same meaning as commonly used in the art to which they pertain. For the purpose of the present invention, the following terms are defined below.

The term “correlatable,” when used relative to breast cancer, indicates that the designated subject, e.g., a polymorphic nucleic acid or the expression or activity of an expression product, is statistically associated with breast cancer.

The term “nucleic acid” is generally used in its art-recognized meaning to refer to a ribose nucleic acid (RNA) or deoxyribose nucleic acid (DNA) polymer or analog thereof, e.g., a nucleotide polymer comprising modifications of the nucleotides, a peptide nucleic acid (PNA), or the like. In certain applications, the nucleic acid can be a polymer that includes both RNA and DNA subunits. A nucleic acid can be, e.g., a chromosome or chromosomal segment, a vector (e.g., an expression vector), a naked DNA or RNA polymer, the product of a polymerase chain reaction (PCR), an oligonucleotide, a probe, etc.

The term “polynucleotide sequence” refers to a contiguous sequence of nucleotides in a nucleic acid or to a representation, e.g., a character string, thereof, depending on context. “Polymorphic polynucleotides” are polynucleotide sequences corresponding to a single locus, i.e., alleles at a locus, characterized by at least one variant (or alternative) nucleotide subunit. Thus, a polymorphic polynucleotide is a polynucleotide that differs, e.g., from another allele at the same locus, or between an otherwise homologous or similar polynucleotide, at one or more nucleotide positions.

The term “unique nucleotides” refers to a polynucleotide sequence corresponding to a unique locus, e.g., a non-repetitive, or unduplicated, locus in the human genome.

An “expression vector” is a vector, e.g., a plasmid, capable of producing transcripts and, potentially, polypeptides encoded by a polynucleotide sequence. Typically, an expression vector is capable of producing transcripts in an exogenous cell, e.g., a bacterial cell, a mammalian cultured cell, or a mammalian cell. Expression of a product can be either constitutive or inducible depending, e.g., on the promoter selected. In the context of an expression vector, a promoter is said to be “operably linked” to a polynucleotide sequence if it is capable of regulating expression of the associated polynucleotide sequence. The term also applies to alternative exogenous gene constructs, such as expressed or integrated transgenes. Similarly, the term operably linked applies equally to alternative or additional transcriptional regulatory sequences such as enhancers, associated with a polynucleotide sequence.

An “expression product” is a transcribed sense or antisense RNA (e.g., an mRNA or an nRNA), or a translated polypeptide corresponding to or derived from a polynucleotide sequence. Depending on the context, the term also can be used to refer to an amplification product (amplicon) or cDNA corresponding to the RNA expression product transcribed from the polynucleotide sequence.

A polynucleotide sequence is said to “encode” a sense or antisense RNA molecule, or a polypeptide, if the polynucleotide sequence can be transcribed (in spliced or unspliced form) or translated into the RNA or into a polypeptide, or a fragment thereof.

A probe and a gene (or expression product) are said to “correspond” when they share substantial structural identity or complimentary, depending on the context. For example, a probe or an expression product, e.g., a messenger RNA, corresponds to a gene when it is derived from a genetic element with substantial sequence identity.

An antibody refers to a protein that comprises one or more polypeptides substantially or partially encoded by immunoglobulin genes or fragments of immunoglobulin genes. The term “antibody,” as used herein includes antibody fragments either produced by the modification of whole antibodies or synthesized de novo using molecular biology techniques. Antibodies include single chain antibodies, including single chain Fv (sFv) antibodies in which a variable heavy and a variable light chain are joined together (directly or through a peptide linker) to form a continuous polypeptide.

The term “subject” as used herein includes, but is not limited to, an organism; a mammal, including, e.g., a human, non-human primate (e.g., monkey), mouse, pig, cow, goat, rabbit, rat, guinea pig, hamster, horse, monkey, sheep, or other non-human mammal.

The term “pharmaceutical composition” means a composition suitable for pharmaceutical use in a subject, including an animal or human. A pharmaceutical composition generally comprises an effective amount of an active agent and a pharmaceutically acceptable excipient or carrier.

The term “effective amount” means a dosage or amount sufficient to produce a desired result. The desired result can comprise an objective or subjective improvement in the recipient of the dosage or amount.

A prophylactic treatment is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A prophylactic activity is an activity of an agent, such as a nucleic acid, vector, gene, polypeptide, protein, substance, or composition thereof that, when administered to a subject who does not display signs or symptoms of pathology, disease or disorder, or who displays only early signs or symptoms of pathology, disease, or disorder, diminishes, prevents, or decreases the risk of the subject developing a pathology, disease, or disorder. A prophylactically useful agent or compound (e.g., nucleic acid or polypeptide) refers to an agent or compound that is useful in diminishing, preventing, treating, or decreasing development of pathology, disease or disorder.

A therapeutic treatment is a treatment administered to a subject who displays symptoms or signs of pathology, disease, or disorder, in which treatment is administered to the subject for the purpose of diminishing or eliminating those signs or symptoms of pathology, disease, or disorder. A therapeutic activity is an activity of an agent, such as a nucleic acid, vector, gene, polypeptide, protein, substance, or composition thereof, that eliminates or diminishes signs or symptoms of pathology, disease or disorder, when administered to a subject suffering from such signs or symptoms. A therapeutically useful agent or compound (e.g., nucleic acid or polypeptide or peptide) indicates that an agent or compound is useful in diminishing, treating, or eliminating such signs or symptoms of a pathology, disease or disorder.

### Polynucleotides of the Invention

The present invention is based on the identification and isolation of a set of differentially expressed genes associated with breast cancer. The unique utility of these polynucleotide sequences, designated herein SEQ ID NO: 1 through SEQ ID NO: 491, resides in their satisfaction of the following criteria. SEQ ID NO: 1-491 are implicated in the transformation and/or progression of a normal mammary epithelia to breast cancer by their differential regulation in normal mammary epithelial cells, compared to breast cancer cells. For example, SEQ ID NO: 1-286 can be used to distinguish ER+ breast cancer cells from ER− breast cancer cells and/or are implicated in the progression of an ER+ breast cancer cell to an ER− breast cancer cell by their differential regulation in ER+ breast cancer cells compared to ER− breast cancer cells. The specified sequences can also correspond to loci on chromosomes involved in breast cancer. For example, SEQ ID NO: 1 corresponds to loci on human chromosome 11 in a narrowly delimited chromosomal region, 11q13→q14, in which a breast cancer locus is found. That the specified sequences satisfy these independent and distinct conditions confers certain unique descriptive and marker utilities, individually and collectively, on the polynucleotide sequence of the invention.

Accordingly, in one aspect, the polynucleotide sequences of the invention are useful for identifying corresponding cDNAs associated with breast cancer, corresponding cDNAs associated with a type of breast cancer and/or chromosomal segments associated with breast cancer. More generally, the polynucleotide sequences of the invention and corresponding polypeptides are useful, individually and/or collectively, as probes (e.g., probes labeled with a detectable moiety) and markers. Such probes and markers are useful not only for identifying breast cancer genes, but also for evaluating breast cancer susceptibility (e.g., for diagnostic assays for determining breast cancer susceptibility in a subject, such as a human subject, or patient) and responsiveness to certain treatment(s), e.g., hormonal treatment. In addition, the polynucleotide sequences of the invention are useful for the production of animal and cell culture models useful for the evaluation of monitoring of therapeutic agents and protocols aimed at treating breast cancer.

Polynucleotide sequences of the invention include, e.g., the polynucleotide sequences represented by SEQ ID NO: 1 through SEQ ID NO: 491. In addition to the sequences expressly provided in the accompanying sequence listing, polynucleotide sequences that are highly related both structurally and functionally are polynucleotides of the invention. For example, polynucleotides encoding a polypeptide having a sequence or subsequence encoded by SEQ ID NO: 1 through SEQ ID NO: 491, or subsequences thereof are one embodiment of the invention. In addition, polynucleotide sequences of the invention include polynucleotide sequences that hybridize under stringent conditions to a polynucleotide sequence comprising any of SEQ ID NO: 1-SEQ ID NO: 491.

In addition to the polynucleotide sequences of the invention, e.g., enumerated in SEQ ID NO: 1 to SEQ ID NO: 491, polynucleotide sequences that are substantially identical to a polynucleotide of the invention can be used in the compositions and methods of the invention. Substantially identical, or substantially similar polynucleotide (or polypeptide) sequences are defined as polynucleotide (or polypeptide) sequences that are identical, on a nucleotide by nucleotide basis, with at least a subsequence of a reference polynucleotide (or polypeptide), e.g., selected from SEQ ID NO: 1-491 (or 492). Such polynucleotides can include, e.g., insertions, deletions, and substitutions relative to any of SEQ ID NO: 1-491. For example, such polynucleotides are typically at least about 70% identical to a reference polynucleotide (or polypeptide) selected from among SEQ ID NO: 1 through SEQ ID NO: 491 (492). That is, at least 7 out of 10 nucleotides (or amino acids) within a window of comparison are identical to the reference sequence selected SEQ ID NO: 1-491 (492). Frequently, such sequences are at least about 80%, usually at least about 90%, and often at least about 95%, or even at least about 98%, or about 99%, identical to the reference sequence, e.g., at least one of SEQ ID NO: 1 to SEQ ID NO: 491.

Subsequences of the polynucleotides of the invention described above, e.g., SEQ ID Nos: 1-491, including at least about 10 contiguous nucleotides or complementary subsequences thereof are also a feature of the invention. More commonly a subsequence includes at least about 12 contiguous nucleotides, e.g., of one or more of SEQ ID NO: 1 through SEQ ID NO: 491. Typically, the subsequence includes at least about 14, frequently at least about 16, and usually at least about 17 or more contiguous nucleotides of one of the specified polynucleotide sequences. Such subsequences can be, e.g., oligonucleotides, such as synthetic oligonucleotides, or full-length genes or cDNAs.

Additionally, the polynucleotides sequences of the invention include polynucleotide sequences that are proximally linked in the human genome to any one of SEQ ID NO: 1 through SEQ ID NO: 491. In the context of the invention, the term “proximally linked” or “linked” is used to indicate that the sequences reside on the same physical nucleic acid. Most typically, the nucleic acid is an expression product, such as a full-length cDNA, or chromosomal segment including the coding domain of an expression product. Using well-known procedures such as genome or chromosome walking (using molecular or bioinformatic approaches), it is a routine matter to identify and isolate such linked nucleic acids. Chromosome walking (and jumping procedures) are well known in the art and are further described, e.g., in Poustka et al. (1987) Construction and use of human chromosome jumping libraries from NotI-digested DNA Nature 325:353-5; Jones et al. (1993) Genome walking with 2- to 4-kb steps using panhandle PCR PCR Methods Appl 2:197-203; Shyamala and Ames (1989) Genome walking by single-specific primer polymerase chain reaction: SSP-PCR Gene 84:1-8; Kere et al. (1992) Mapping human chromosomes by walking with sequence-tagged sites from end fragments of yeast artificial chromosome inserts Genomics 14:241-8; Sandford and Elgar (1992) A novel methodfor rapid genomic walking using lambda vectors Nucleic Acids Res 20:4665-6; and, Cross and Little (1986) A cosmid vectorfor systematic chromosome walking Gene 49: 9-22.

For example, as described in further detail below, labeled probes corresponding to any one or more of SEQ ID NO: 1-491 can be used to screen expression (e.g., cDNA) or genomic (e.g., chromosomal) libraries to identify expression products or genomic segments that include adjacent polynucleotide sequences along with the polynucleotide sequence hybridizing to the probe selected from SEQ ID NO: 1 to SEQ ID NO: 491. Such linked polynucleotide sequences are also a feature of the invention and are useful in the methods and compositions described herein.

Polynucleotides encoding polypeptides having amino acids sequences or subsequences encoded by SEQ ID NOs: 1-491 are also an embodiment of the invention. Subsequences of SEQ ID NO: 1-491 including at least about 10 contiguous nucleotides or complementary subsequences are also a feature of the invention. More commonly a subsequence includes, e.g., at least about 12 contiguous nucleotides of one or more of SEQ ID NO: 1 through SEQ ID NO: 491. Typically, the subsequence includes at least about 14, frequently at least about 16, and usually at least about 17 contiguous nucleotides of one of the specified polynucleotide sequences. Such subsequences are typically oligonucleotides, such as synthetic oligonucleotides.

In addition, polynucleotide sequences complementary to any of the above described sequences are included among the polynucleotide sequences of the invention.

Where the polynucleotide sequences are translated to form a polypeptide or subsequence of a polypeptide, the nucleotide changes can result in either conservative or non-conservative amino acid substitutions. Conservative amino acid substitutions refer to the interchangeability of residues having functionally similar side chains. Conservative substitution tables providing functionally similar amino acids are well known in the art. Table 1 sets forth six groups that contain amino acids that are “conservative substitutions” for one another. Other conservative substitution charts are available in the art, and can be used in a similar manner.

TABLE 1
Conservative Substitution Groups

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (Q) | | |
| 4 | Arginine (R) | Lysine (K) | | |
| 5 | Isoleucine (I) | Leucine (L) | Methionine (M) | Valine (V) |
| 6 | Phenylalanine (F) | Tyrosine (Y) | Tryptophan (W) | |

One of skill will appreciate that many conservative variations of the nucleic acid constructs, which are disclosed, yield a functionally identical construct. For example, as discussed above, owing to the degeneracy of the genetic code, “silent substitutions” (i.e., substitutions in a nucleic acid sequence which do not result in an alteration in an encoded polypeptide) are an implied feature of every nucleic acid sequence that encodes an amino acid. Similarly, “conservative amino acid substitutions,” in one or a few amino acids in an amino acid sequence (e.g., about 1%, about 2%, about 3%, about 4%, about 5%, about 6%, about 7%, about 8%, about 9%, about 10% or more) are substituted with different amino acids with highly similar properties, are also readily identified as being highly similar to a disclosed construct. Such conservative variations of each disclosed sequence are a feature of the present invention.

Methods for obtaining conservative variants, as well as more divergent versions of the nucleic acids and polypeptides of the invention are widely known in the art. In addition to naturally occurring homologues which can be obtained, e.g., by screening genomic or expression libraries according to any of a variety of well-established protocols, see, e.g., Ausubel et al. Current Protocols in Molecular Biology (supplemented through 2001) John Wiley & Sons, New York (“Ausubel”); Sambrook et al. Molecular Cloning-A Laboratory Manual (2nd Ed.), Vol. 1-3, Cold Spring Harbor Laboratory, Cold Spring Harbor, N.Y, 1989 (“Sambrook”), and Berger and Kimmel Guide to Molecular Cloning Techniques, Methods in Enzymology volume 152 Academic Press, Inc., San Diego, Calif. (“Berger”), additional variants can be produced by a variety of mutagenesis procedures. Many such procedures are known in the art, including site directed mutagenesis, oligonucleotide-directed mutagenesis, and many others. For example, site directed mutagenesis is described, e.g., in Smith (1985) “In vitro mutagenesis” Ann. Rev. Genet. 19:423-462, and references therein, Botstein & Shortle (1985) “Strategies and applications of in vitro mutagenesis” Science 229:1193-1201; and Carter (1986) “Site-directed mutagenesis” Biochem. J. 237:1-7. Oligonucleotide-directed mutagenesis is described, e.g., in Zoller & Smith (1982) “Oligonucleotide-directed mutagenesis using M13-derived vectors: an efficient and general procedure for the production of point mutations in any DNA fragment” Nucleic Acids Res. 10:6487-6500). Mutagenesis using modified bases is described e.g., in Kunkel (1985) “Rapid and efficient site-specific mutagenesis without phenotypic selection” Proc. Natl. Acad. Sci. USA 82:488-492, and Taylor et al. (1985) “The rapid generation of oligonucleotide-directed mutations at high frequency using phosphorothioate-modified DNA” Nucl. Acids Res. 13: 8765-8787. Mutagenesis using gapped duplex DNA is described, e.g., in Kramer et al. (1984) “The gapped duplex DNA approach to oligonucleotide-directed mutation construction” Nucl. Acids Res. 12: 9441-9456). Point mismatch repair is described, e.g., by Kramer et al. (1984) “Point Mismatch Repair” Cell 38:879-887). Double-strand break repair is described, e.g., in Mandecki (1986) “Oligonucleotide-directed double-strand break repair in plasmids of Escherichia coli: a method for site-specific mutagenesis” Proc. Natl. Acad. Sci. USA, 83:7177-7181, and in Arnold (1993) “Protein engineering for unusual environments” Current Opinion in Biotechnology 4:450-455). Mutagenesis using repair-deficient host strains is described, e.g., in Carter et al. (1985) “Improved oligonucleotide site-directed mutagenesis using M13 vectors” Nucl. Acids Res. 13: 4431-4443. Mutagenesis by total gene synthesis is described e.g., by Nambiar et al. (1984) “Total synthesis and cloning of a gene coding for the ribonuclease S protein” Science 223: 1299-1301. DNA shuffling is described, e.g., by Stemmer (1994) “Rapid evolution of a protein in vitro by DNA shuffling” Nature 370:389-391, and Stemmer (1994) “DNA shuffling by random fragmentation and reassembly: In vitro recombination for molecular evolution.” Proc. Natl. Acad. Sci. USA 91:10747-10751.

Many of the above methods are further described in Methods in Enzymology Volume 154, which also describes useful controls for trouble-shooting problems with various mutagenesis methods. Kits for mutagenesis, library construction and other diversity generation methods are also commercially available. For example, kits are available from, e.g., Amersham International plc (e.g., using the Eckstein method above), Anglian Biotechnology Ltd (e.g., using the Carter/Winter method above), Bio/Can Scientific, Bio-Rad (e.g., using the Kunkel method described above), Boehringer Mannheim Corp., Clonetech Laboratories, DNA Technologies, Epicentre Technologies (e.g., the 5 prime 3 prime kit); Genpak Inc, Lemargo Inc, Life Technologies (Gibco BRL), New England Biolabs, Pharmacia Biotech, Promega Corp., Quantum Biotechnologies, Stratagene (e.g., QuickChange™ site-directed mutagenesis kit; and Chameleon™ double-stranded, site-directed mutagenesis kit).

### Determining Sequence Relationships

A variety of methods for determining relationships between two or more sequences (e.g., identity, similarity and/or homology) are available, and well known in the art. The methods include manual alignment and computer assisted sequence alignment and analysis. A number of algorithms for performing sequence alignment are widely available, or can be produced by one of skill, including: the local homology algorithm of Smith and Waterman (1981) Adv. Appl. Math. 2:482; the homology alignment algorithm of Needleman and Wunsch (1970) J. Mol. Biol. 48:443; the search for similarity method of Pearson and Lipman (1988) Proc. Natl. Acad. Sci. (USA) 85:2444; and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.).

For example, software for performing sequence identity (and sequence similarity) analysis using the BLAST algorithm, described in Altschul et al. (1990) J. Mol. Biol. 215:403-410, is publicly available through the National Center for Biotechnology Information (on the World Wide Web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=-4, and a comparison of both strands. For amino acid sequences, the BLASTP program uses as defaults a wordlength (W) of 3, an expectation (E) of 10, and the BLOSUM62 scoring matrix (see, Henikoff & Henikoff (1989) Proc. Natl. Acad. Sci. USA 89:10915).

Additionally, the BLAST algorithm performs a statistical analysis of the similarity between two sequences (see, e.g., Karlin & Altschul (1993) Proc. Nat'l. Acad. Sci. USA 90:5873-5787). One measure of similarity provided by the BLAST algorithm is the smallest sum probability (p(N)), which provides an indication of the probability by which a match between two nucleotide or amino acid sequences would occur by chance. For example, a nucleic acid is considered similar to a reference sequence (and, therefore, in this context, homologous) if the smallest sum probability in a comparison of the test nucleic acid to the reference nucleic acid is less than about 0.1, or less than about 0.01, and or even less than about 0.001.

Another example of a useful sequence alignment algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS5:151-153. The program can align, e.g., up to 300 sequences of a maximum length of 5,000 letters. The multiple alignment procedure begins with the pairwise alignment of the two most similar sequences, producing a cluster of two aligned sequences. This cluster can then be aligned to the next most related sequence or cluster of aligned sequences. Two clusters of sequences can be aligned by a simple extension of the pairwise alignment of two individual sequences. The final alignment is achieved by a series of progressive, pairwise alignments. The program can also be used to plot a dendogram or tree representation of clustering relationships. The program is run by designating specific sequences and their amino acid or nucleotide coordinates for regions of sequence comparison.

An additional example of an algorithm that is suitable for multiple DNA (or amino acid) sequence alignments is the CLUSTALW program (Thompson, J. D. et al. (1994) Nucl. Acids. Res. 22: 4673-4680). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff (1992) Proc. Natl. Acad. Sci. USA 89: 10915-10919).

Nucleic Acid Hybridization

Similarity between nucleic acids can also be evaluated by “hybridization” between single stranded (or single stranded regions of) nucleic acids with complementary or partially complementary polynucleotide sequences. Hybridization is a measure of the physical association between nucleic acids, typically, in solution, or with one of the nucleic acid strands immobilized on a solid support, e.g., a membrane, a bead, a chip, a filter, etc. Nucleic acid hybridization occurs based on a variety of well characterized physico-chemical forces, such as hydrogen bonding, solvent exclusion, base stacking and the like. Numerous protocols for nucleic acid hybridization are well known in the art. An extensive guide to the hybridization of nucleic acids is found in Tijssen (1993) Laboratory Techniques in Biochemistry and Molecular Biology—Hybridization with Nucleic Acid Probes, part I, chapter 2, “Overview of principles of hybridization and the strategy of nucleic acid probe assays,” (Elsevier, N.Y.), as well as in Ausubel, supra, Sambrook, supra and Berger, supra. Hames and Higgins (1995) Gene Probes 1, IRL Press at Oxford University Press, Oxford, England (“Hames and Higgins 1”) and Hames and Higgins (1995) Gene Probes 2, IRL Press at Oxford University Press, Oxford, England (“Hames and Higgins 2”) provide details on the synthesis, labeling, detection and quantification of DNA and RNA, including oligonucleotides.

Conditions suitable for obtaining hybridization, including differential hybridization, are selected according to the theoretical melting temperature (Tₘ) between complementary and partially complementary nucleic acids. Under a given set of conditions, e.g., solvent composition, ionic strength, etc., the Tₘ is the temperature at which the duplex between the hybridizing nucleic acid strands is 50% denatured. That is, the Tₘ corresponds to the temperature corresponding to the midpoint in transition from helix to random coil; it depends on length, nucleotide composition, and ionic strength for long stretches of nucleotides.

After hybridization, unhybridized nucleic acids can be removed by a series of washes, the stringency of which can be adjusted depending upon the desired results. Low stringency washing conditions (e.g., using higher salt and lower temperature) increase sensitivity, but can produce nonspecific hybridization signals and high background signals. Higher stringency conditions (e.g., using lower salt and higher temperature that is closer to the hybridization temperature) lower the background signal, typically with only the specific signal remaining. See, Rapley, R. and Walker, J. M. eds., Molecular Biomethods Handbook (Humana Press, Inc. 1998).

“Stringent hybridization wash conditions” or “stringent conditions” in the context of nucleic acid hybridization experiments, such as Southern and northern hybridizations, are sequence dependent, and are different under different environmental parameters. An extensive guide to the hybridization of nucleic acids is found in Tijssen (1993), supra, and in Hames and Higgins 1 and Hames and Higgins 2, supra.

An example of stringent hybridization conditions for hybridization of complementary nucleic acids which have more than 100 complementary residues on a filter in a Southern or northern blot is 2×SSC, 50% formamide at 42° C., with the hybridization being carried out overnight (e.g., for approximately 20 hours). An example of stringent wash conditions is a 0.2×SSC wash at 65° C. for about 15 minutes (see Sambrook, supra for a description of SSC buffer). Often the wash determining the stringency is preceded by a low stringency wash to remove signal due to residual unhybridized probe. An example low stringency wash is 2×SSC at room temperature (e.g., 20° C. for about 15 minutes).

In general, a signal to noise ratio of at a level of 2.5×-5× (and typically higher) than that observed for an unrelated probe in the particular hybridization assay indicates detection of a specific hybridization. Detection of at least stringent hybridization between two sequences in the context of the present invention indicates relatively strong structural similarity to, e.g., the nucleic acids of the present invention provided in the sequence listings herein.

For purposes of the present invention, generally, “highly stringent” hybridization and wash conditions are selected to be about 5° C. or less, lower than the thermal melting point (Tₘ) for the specific sequence at a defined ionic strength and pH (as noted below, highly stringent conditions can also be referred to in comparative terms). Target sequences that are closely related or identical to the nucleotide sequence of interest (e.g., “probe”) can be identified under stringent or highly stringent conditions. Lower stringency conditions are appropriate for sequences that are less complementary.

For example, in determining stringent or highly stringent hybridization (or even more stringent hybridization) and wash conditions, the hybridization and wash conditions are gradually increased (e.g., by increasing temperature, decreasing salt concentration, increasing detergent concentration and/or increasing the concentration of organic solvents, such as formamide, in the hybridization or wash), until selected sets of criteria are met. For example, the hybridization and wash conditions are gradually increased until a probe comprising one or more polynucleotide sequences of the invention, e.g., selected from SEQ ID NO: 1 to SEQ ID NO: 491, and/or complementary polynucleotide sequences thereof, binds to a perfectly matched complementary target (again, a nucleic acid comprising one or more nucleic acid sequences or subsequences selected from SEQ ID NO: 1 to SEQ ID NO: 491, and complementary polynucleotide sequences thereof), with a signal to noise ratio that is at least 2.5×, and optionally 5× or 10× or 100× or more as high as that observed for hybridization of the probe to an unmatched target, as desired.

Using the polynucleotides of the invention, or subsequences thereof, novel target nucleic acids can be obtained, such target nucleic acids are also a feature of the present invention. For example, such target nucleic acids include sequences that hybridize under stringent conditions to an oligonucleotide probe that encodes a unique subsequence in any of the polypeptides of the invention, e.g., SEQ ID NO: 492 or encoded by a sequence selected from SEQ ID NOS: 1-491.

For example, hybridization conditions are chosen under which a target oligonucleotide that is perfectly complementary to the oligonucleotide probe hybridizes to the probe with at least about a 5-10× higher signal to noise ratio than for hybridization of the target oligonucleotide to a control nucleic acid, e.g., a nucleic acid that is not a polynucleotide sequence of the invention (e.g., sequences unrelated to any one of SEQ ID NO: 1-SEQ ID NO: 491).

Higher ratios of signal to noise can be achieved by increasing the stringency of the hybridization conditions such that ratios of about 15×, 20×, 30×, 50× or more are obtained. The particular signal will depend on the label used in the relevant assay, e.g., a fluorescent label, a colorimetric label, a radio active label, or the like.

### Probes

Nucleic acids including one or more polynucleotide sequence of the invention are favorably used as probes for the detection of corresponding or related nucleic acids in a variety of contexts, such as the nucleic hybridization experiments discussed above. The probes can be either DNA or RNA molecules, such as restriction fragments of genomic or cloned DNA, cDNAs, amplification products, transcripts, and oligonucleotides, and can vary in length from oligonucleotides as short as about 10 nucleotides in length to chromosomal fragments or cDNAs in excess of 1 kb or more. For example, in some embodiments, a probe of the invention includes a polynucleotide sequence or subsequence selected from among SEQ ID NO: 1 to SEQ ID NO: 491, or sequences complementary thereto. Alternatively, polynucleotide sequences that are variants of one of the above-designated sequences are used as probes. Most typically, such variants include one or a few conservative nucleotide variations. For example, pairs (or sets) of oligonucleotides can be selected, in which the two (or more) polynucleotide sequences are conservative variations of each other, wherein one polynucleotide sequence correspond identically to a first allele or allelic variant and the other(s) correspond identically to additional alleles or allelic variants. Such pairs of oligonucleotide probes are particularly useful, e.g., for allele specific hybridization experiments to detect polymorphic nucleotides. In other applications, probes are selected that are more divergent, that is probes that are at least about 70% (or about 80%, about 90%, about 95%, about 98%, or about 99%) identical are selected.

The probes of the invention, e.g., as exemplified by sequences derived from SEQ ID NO: 1 through SEQ ID NO: 491, can also be used to identify additional useful polynucleotide sequences according to procedures routine in the art. In one set of embodiments, one or more probes, as described above, are utilized to screen libraries of expression products or chromosomal segments (e.g., expression libraries or genomic libraries) to identify clones that include sequences identical to, or with significant sequence similarity to, one or more of SEQ ID NO: 1-491, i.e., allelic variants, homologues or orthologues. In turn, each of these identified sequences can be used to make probes, including pairs or sets of variant probes as described above. It will be understood that in addition to such physical methods as library screening, computer assisted bioinformatic approaches, e.g., BLAST and other sequence homology search algorithms, and the like, can also be used for identifying related polynucleotide sequences. Polynucleotide sequences identified in this manner are also a feature of the invention.

For example, oligonucleotide probes, most typically produced by well known synthetic methods, such as the solid phase phosphoramidite triester method described by Beaucage and Caruthers (1981) Tetrahedron Letts. 22(20):1859-1862, e.g., using an automated synthesizer, as described in Needham-VanDevanter et al. (1984) Nucleic Acids Res., 12:6159-6168. Oligonucleotides can also be custom made and ordered from a variety of commercial sources known to persons of skill. Purification of oligonucleotides, where necessary, is typically performed by either native acrylamide gel electrophoresis or by anion-exchange HPLC as described in Pearson and Regnier (1983) J. Chrom. 255:137-149. The sequence of the synthetic oligonucleotides can be verified using the chemical degradation method of Maxam and Gilbert (1980) in Grossman and Moldave (eds.) Academic Press, New York, Methods in Enzymology 65:499-560. Custom oligos can also easily be ordered from a variety of commercial sources known to persons of skill.

In addition, essentially any nucleic acid can be custom ordered from any of a variety of commercial sources, such as The Midland Certified Reagent Company (on the World Wide Web at mcrc.com), The Great American Gene Company (on the World Wide Web at genco.com), ExpressGen Inc. (on the World Wide Web at expressgen.com), Operon Technologies, Inc. (Alameda, Calif.) and many others. Similarly, peptides and antibodies can be custom ordered from any of a variety of sources, such as PeptidoGenic (available at pkim@ccnet.com), HTI Bio-products, inc. (on the World Wide Web at htibio.com), BMA Biomedicals Ltd (U.K.), Bio.Synthesis, Inc., and many others.

As noted in one embodiment, oligonucleotide probes of the invention include subsequences of SEQ ID NO: 1 through SEQ ID NO: 491, and/or complementary sequences thereof including at least about 10 contiguous nucleotides in length. Commonly, the oligonucleotide probes are at least about 12 contiguous nucleotides in length; usually, the oligonucleotides are at least about 14 contiguous nucleotides in length; frequently, the oligonucleotides are at least about 16 contiguous nucleotides in length, and in many cases the oligonucleotides are at least about 17 or more contiguous nucleotides of at least one sequence selected from SEQ ID NO: 1 to SEQ ID NO: 491. In some cases, the oligonucleotide probes consist of a polynucleotide sequence selected from SEQ ID NO: 1 through SEQ ID NO: 491.

In other circumstances, e.g., relating to functional attributes of cells or organisms expressing the polynucleotides and polypeptides of the invention, probes that are polypeptides, peptides or antibodies are favorably utilized. For example, isolated or recombinant polypeptides, polypeptide fragments and peptides derived from, e.g., SEQ ID NO: 492 and/or encoded by polynucleotide sequences of the invention, e.g., selected from SEQ ID NO: 1 to SEQ ID NO: 491, are favorably used to identify and isolate antibodies or other binding proteins, e.g., from phage display libraries, combinatorial libraries, polyclonal sera, and the like.

Antibodies specific for any a polypeptide sequence or subsequence, e.g., of SEQ ID NO: 492, and/or encoded by polynucleotide sequences of the invention, e.g., selected from SEQ ID NO: 1 to SEQ ID NO: 491, are likewise valuable as probes for evaluating expression products, e.g., from cells or tissues. In addition, antibodies are particularly suitable for evaluating expression of proteins comprising amino acid subsequences, e.g., of SEQ ID NO: 492, or encoded by polynucleotides sequences of the invention, e.g., selected from SEQ ID Nos.1-491, in situ, in a tissue array, in a cell, tissue or organism, e.g., an organism providing an experimental model of breast cancer. Antibodies can be directly labeled with a detectable reagent as described below, or detected indirectly by labeling of a secondary antibody specific for the heavy chain constant region (i.e., isotype) of the specific antibody. Additional details regarding production of specific antibodies are provided below in the section entitled “Antibodies.”

Labeling and Detecting Probes

Numerous methods are available for labeling and detection of the nucleic acid and polypeptide (or peptide or antibody) probes of the invention, these include: 1) Fluorescence (using, e.g., fluorescein, Cy-5, rhodamine or other fluorescent tags); 2) Isotopic methods, e.g., using end-labeling, nick translation, random priming, or PCR to incorporate radioactive isotopes into the probe polynucleotide/oligonucleotide; 3) Chemifluorescence using Alkaline Phosphatase and the substrate AttoPhos (Amersham) or other substrates that produce fluorescent products; 4) Chemi luminescence (using either Horseradish Peroxidase and/or Alkaline Phosphatase with substrates that produce photons as breakdown products, kits providing reagents and protocols are available from such commercial sources as Amersham, Boehringer-Mannheim, and Life Technologies/Gibco BRL); and, 5) Colorimetric methods (again using both Horseradish Peroxidase and Alkaline Phosphatase with substrates that produce a colored precipitate, kits are available from Life Technologies/Gibco BRL, and Boehringer-Mannheim). Other methods for labeling and detection will be readily apparent to one skilled in the art.

More generally, a probe can be labeled with any composition detectable by spectroscopic, photochemical, biochemical, immunochemical, electrical, optical or chemical means. Useful labels in the present invention include spectral labels such as fluorescent dyes (e.g., fluorescein isothiocyanate, Texas red, rhodamine, and the like), radiolabels (e.g., ³H, ¹²⁵I, ³⁵S, ¹⁴C, ³²P, ³³P, etc.), enzymes (e.g., horse-radish peroxidase, alkaline phosphatase, etc.), spectral calorimetric labels such as colloidal gold or colored glass or plastic (e.g. polystyrene, polypropylene, latex, etc.) beads. The label can be coupled directly or indirectly to a component of the detection assay (e.g., a probe, such as an oligonucleotide, isolated DNA, amplicon, restriction fragment, or the like) according to methods well known in the art. As indicated above, a wide variety of labels can be used, with the choice of label depending on sensitivity required, ease of conjugation with the compound, stability requirements, available instrumentation, and disposal provisions. In general, a detector which monitors a probe-target nucleic acid hybridization is adapted to the particular label which is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising a nucleic acid array with particular set of probes bound to the array is digitized for subsequent computer analysis.

Because incorporation of radiolabeled nucleotides into nucleic acids is straightforward, this detection represents one favorable labeling strategy. Exemplar technologies for incorporating radiolabels include end-labeling with a kinase or phoshpatase enzyme, nick translation, incorporation of radio-active nucleotides with a polymerase and many other well-known strategies.

Fluorescent labels are desirable, having the advantage of requiring fewer precautions in handling, and being amenable to high-throughput visualization techniques. Typically, labels are characterized by one or more of the following: high sensitivity, high stability, low background, low environmental sensitivity and high specificity in labeling. Fluorescent moieties, which are incorporated into the labels of the invention, are generally are known, including Texas red, fluorescein isothiocyanate, rhodamine, etc. Many fluorescent tags are commercially available from SIGMA chemical company (Saint Louis, Mo.), Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica- Biochemika Analytika (Fluka Chemie AG, Buchs, Switzerland), and Applied Biosystems (Foster City, Calif.) as well as other commercial sources known to one of skill. Similarly, moieties such as digoxygenin and biotin, which are not themselves fluorescent but are readily used in conjunction with secondary reagents, i.e., anti-digoxygenin antibodies, avidin (or streptavidin), that can be labeled, are suitable as labeling reagents in the context of the probes of the invention.

The label is coupled directly or indirectly to a molecule to be detected (a product, substrate, enzyme, or the like) according to methods well known in the art. As indicated above, a wide variety of labels are used, with the choice of label depending on the sensitivity required, ease of conjugation of the compound, stability requirements, available instrumentation, and disposal provisions. Non-radioactive labels are often attached by indirect means. Generally, a ligand molecule (e.g., biotin) is covalently bound to a nucleic acid such as a probe, primer, amplicon, or the like. The ligand then binds to an anti-ligand (e.g., streptavidin) molecule, which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with labeled, anti-ligands. Alternatively, any haptenic or antigenic compound can be used in combination with an antibody. Labels can also be conjugated directly to signal generating compounds, e.g., by conjugation with an enzyme or fluorophore or chromophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. Means of detecting labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems that are widely available.

It will be appreciated that probe design is influenced by the intended application. For example, where several allele-specific probe-target interactions are to be detected in a single assay, e.g., on a single DNA chip, it is desirable to have similar melting temperatures for all of the probes. Accordingly, the length of the probes are adjusted so that the melting temperatures for all of the probes on the array are closely similar (it will be appreciated that different lengths for different probes may be needed to achieve a particular Tₘ where different probes have different GC contents). Although melting temperature is a primary consideration in probe design, other factors are optionally used to further adjust probe construction, such as selecting against primer self-complementarily and the like.

### Marker Sets

Sets of probes, including multiple nucleic acids with polynucleotide sequences selected from among the polynucleotide sequences of the invention, e.g., SEQ ID NO: 1 through SEQ ID NO: 491, are also a feature of the invention. Such sets of probes are useful as marker sets, e.g., for predicting breast cancer, for predicting at least one characteristic of a breast cell, identifying phenotypes and the like.

Marker sets of the invention favorably include any of the probe sequences described above, such as polynucleotide sequences that hybridize under stringent conditions to any one of SEQ ID NO: 1-SEQ ID NO: 491, sequences that are at least about 70% identical to one or more of SEQ ID NO: 1 through SEQ ID NO: 491, sequences that encode a polypeptide or peptide comprising a subsequence encoded by any one of SEQ ID NO: 1-SEQ ID NO: 491, sequences that are physically linked in the human genome to any one of SEQ ID NO: 1-SEQ ID NO: 491, as well as sequences complementary to any such sequences, or subsequences thereof.

In one embodiment, the marker set of the invention is a plurality of oligonucleotides, e.g., synthetic oligonucleotides produced by the phosporamidite triester synthesis method on an automated synthesizer, as described above. For example, at least two oligonucleotides including a polynucleotide sequence of at least about 10 contiguous nucleotides of a polynucleotide of the invention, e.g., selected from SEQ ID NO: 1 to SEQ ID NO: 491, can be used as a set to predict breast cancer. Frequently, the oligonucleotides selected will be longer than about 10 contiguous nucleotides in length, for example, oligonucleotides of at least about 12, or about 14, or about 16 or about 17, or more contiguous nucleotides are favorably employed in the marker sets of the invention.

While as few as two probes constitute a marker set, it is frequently desirable to employ marker sets with more than two members. Typically, a marker set of the invention has at least 3, often at least about 5 or more members selected from among any of the polynucleotides of the invention. In one favorable embodiment, the marker set includes oligonucleotides corresponding in sequence to at least part of each of SEQ ID NO: 1 through SEQ ID NO: 491. In one embodiment, the marker set is made up of nucleic acids including polynucleotide sequences corresponding to each of SEQ ID NO: 1 through SEQ ID NO: 491. In another embodiment, each member of the marker set comprises at least about 10 contiguous nucleotides, e.g., selected from SEQ ID NO: 1-SEQ ID NO: 491. In other aspects, the plurality of members of the marker set together comprise a plurality of sequences or subsequences selected from a plurality of nucleic acids represented by the polynucleotides of the invention. In another embodiment, the marker set includes a plurality of members, where a majority of members of the marker set together comprise a majority of subsequences from a majority of the polynucleotides of the invention. In one embodiment, the marker sets are made up of expression products such as cDNAs, or amplification products corresponding to cDNA or RNA expression products.

In some applications, the marker set includes labeled nucleic acid probes as described in the preceding section. In other applications, e.g., certain array applications, a labeled nucleic acid sample is hybridized to a set of unlabeled marker nucleic acids.

The marker sets of the invention are frequently employed in the context of a polynucleotide sequence array. Any of the polynucleotide sequences of the invention, as described above, can be logically or physically arrayed to produce an array. For example, nucleic acids, e.g., oligonucleotides, cDNAs, amplicons, or chromosomal segments, can be physically arrayed in a solid phase or liquid phase array. Common solid phase arrays include a variety of solid substrates suitable for attaching nucleic acids in an ordered manner, such as membranes, filters, chips, beads, pins, slides, plates, etc. Common liquid phase arrays include, e.g., arrays of wells (e.g., as in microtiter trays) or containers (e.g., as in arrays of test tubes).

Nucleic acids of the marker sets are immobilized, for example by direct or indirect cross-linking, to the solid support. Essentially any solid support capable of withstanding the reagents and conditions used in the particular detection assay can be utilized. For example, functionalized glass, silicon, silicon dioxide, modified silicon, any of a variety of polymers, such as (poly)tetrafluoroethylene, (poly)vinylidenedifluoride, polystyrene, polycarbonate, or combinations thereof can all serve as the substrate for a solid phase array.

In one embodiment, the array is a “chip” composed, e.g., of one of the above specified materials. Polynucleotide probes, e.g., RNA or DNA, such as cDNA, synthetic oligonucleotides, and the like, as discussed above are adhered to the chip in a logically ordered manner, i.e., in an array. Additional details regarding methods for linking nucleic acids and proteins to a chip substrate, can be found in, e.g., U.S. Pat. No. 5,143,854 “Large Scale Photolithographic Solid Phase Synthesis of Polypeptides and Receptor Binding Screening Thereof” to Pirrung et al., issued, Sep. 1, 1992; U.S. Pat. No. 5,837,832 “Arrays of Nucleic Acid Probes on Biological Chips” to Chee et al., issued Nov. 17, 1998; U.S. Pat. No. 6,087,112 “Arrays with Modified Oligonucleotide and Polynucleotide Compositions” to Dale, issued Jul. 11, 2000; U.S. Pat. No. 5,215,882 “Method of Immobilizing Nucleic Acid on a Solid Substrate for Use in Nucleic Acid Hybridization Assays” to Bahl et al., issued Jun. 1, 1993; U.S. Pat. No. 5,707,807 “Molecular Indexing for Expressed Gene Analysis” to Kato, issued Jan. 13, 1998; U.S. Pat. No. 5,807,522 “Methods for Fabricating Microarrays of Biological Samples” to Brown et al., issued Sep. 15, 1998; U.S. Pat. No. 5,958,342 “Jet Droplet Device” to Gamble et al., issued Sep. 28, 1999; U.S. Pat. No. 5,994,076 “Methods of Assaying Differential Expression” to Chenchik et al., issued Nov. 30, 1999; U.S. Pat. No. 6,004,755 “Quantitative Microarray Hybridization Assays” to Wang, issued Dec. 21, 1999; U.S. Pat. No. 6,048,695 “Chemically Modified Nucleic Acids and Method for Coupling Nucleic Acids to Solid Support” to Bradley et al., issued Apr. 11, 2000; U.S. Pat. No. 6,060,240 “Methods for Measuring Relative Amounts of Nucleic Acids in a Complex Mixture and Retrieval of Specific Sequences Therefrom” to Kamb et al., issued May 9, 2000; U.S. Pat. No. 6,090,556 “Method for Quantitatively Determining the Expression of a Gene” to Kato, issued Jul. 18, 2000; and U.S. Pat. No. 6,040,138 “Expression Monitoring by Hybridization to High Density Oligonucleotide Arrays” to Lockhart et al., issued Mar. 21, 2000.

In addition to being able to design, build and use probe arrays using available techniques, one of skill is also able to order custom-made arrays and array-reading devices from manufacturers specializing in array manufacture. An example, Affymetrix Corp., in Santa Clara, Calif. manufactures DNA VLSIP™ arrays. Another array manufacture is Agilent Technology, Inc.

In addition to marker sets made up of nucleic acid probes described above, marker sets including polypeptide, peptide, and antibody probes as discussed in the section entitled “Labeled probes” are favorably used in certain applications. As discussed above for individual peptide or polypeptide probes, sets of probes including multiple members encoded by or having subsequences encoded by the polynucleotides of the invention, e.g., SEQ ID NOs: 1-491 and/or having the sequence or subsequence of SEQ ID NO.: 492, or antibodies specific to such sequences can be used in liquid phase, or immobilized as described above with respect to nucleic acid markers.

Marker sets of the invention also include marker sets specific for predicting at least one characteristic of a breast cell, e.g., detecting types of breast cancer cells, e.g., ER− breast cancer cells verses ER+ breast cancer cells using probe sequences described above, such as, e.g., sequences selected from SEQ ID NO: 1-SEQ ID NO: 286, sequences that hybridize under stringent conditions to any one of SEQ ID NO: 1-SEQ ID NO: 286, sequences that are at least about 70% identical to any one of SEQ ID NO: 1-SEQ ID NO: 286, sequences that encode a polypeptide or peptide comprising a subsequence encoded by any one of SEQ ID NO: 1-SEQ ID NO: 286, sequences that are physically linked in the human genome to any one of SEQ ID NO: 1-SEQ ID NO: 286, sequences complementary to any such sequences, or subsequences thereof including at least about 10 contiguous nucleotides of, e.g., SEQ ID NO: 1-SEQ ID NO: 286. Other embodiments of these markers sets are as described above. Other characteristics include, e.g., transformation state, invasiveness, stage of progression, a protein expressed, a protein expressed on the surface of the breast cell and the like. The marker set can be used for predicting any characteristic of a breast cell or breast cancer cell that describes the cell, e.g., genotype, phenotype, cell cycle time, type of growth, behavior to certain agents, e.g., hormones, pharmaceutical agent, etc.

### Vectors, Promoters and Expression Systems

The present invention includes recombinant constructs incorporating one or more of the nucleic acid sequences described above. Such constructs include a vector, for example, a plasmid, a cosmid, a phage, a virus, a bacterial artificial chromosome (BAC), a yeast artificial chromosome (YAC), etc., into which one or more of the polynucleotide sequences of the invention, e.g., comprising any of SEQ ID NO: 1-491, or a subsequence thereof, has been inserted, in a forward or reverse orientation. For example, the inserted nucleic acid can include a chromosomal sequence or cDNA including all or part of at least one of the polynucleotide sequences of the invention, e.g., selected from SEQ ID NO: 1-SEQ ID NO: 491, such as a sequence originating on human chromosome 11 or a cDNA corresponding to an mRNA expression product transcribed from a polynucleotide sequence on human chromosome 11. In one embodiment, the construct further comprises regulatory sequences, including, for example, a promoter, operably linked to the sequence. Large numbers of suitable vectors and promoters are known to those of skill in the art, and are commercially available.

The polynucleotides of the present invention can be included in any one of a variety of vectors suitable for generating sense or antisense RNA, and optionally, polypeptide (or peptide) expression products. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others. Any vector that is capable of introducing genetic material into a cell, and, if replication is desired, which is replicable in the relevant host can be used.

In an expression vector, the polynucleotide sequence of interest is physically arranged in proximity and orientation to an appropriate transcription control sequence (promoter, and optionally, one or more enhancers) to direct mRNA synthesis. That is, the polynucleotide sequence of interest is operably linked to an appropriate transcription control sequence. Examples of such promoters include: LTR or SV40 promoter, E. coli lac or trp promoter, phage lambda PL promoter, and other promoters known to control expression of genes in prokaryotic or eukaryotic cells or their viruses. The expression vector also contains a ribosome binding site for translation initiation, and a transcription terminator. The vector optionally includes appropriate sequences for amplifying expression. In addition, the expression vectors optionally comprise one or more selectable marker genes to provide a phenotypic trait for selection of transformed host cells, such as dihydrofolate reductase or neomycin resistance for eukaryotic cell culture, or such as tetracycline or ampicillin resistance in E. coli.

Additional Expression Elements

Where translation of polypeptide encoded by a nucleic acid comprising a polynucleotide sequence of the invention is desired, additional translation specific initiation signals can improve the efficiency of translation. These signals can include, e.g., an ATG initiation codon and adjacent sequences. In some cases, for example, full-length cDNA molecules or chromosomal segments including a coding sequence incorporating, e.g., a polynucleotide sequence of the invention, a translation initiation codon and associated sequence elements are inserted into the appropriate expression vector simultaneously with the polynucleotide sequence of interest. In such cases, additional translational control signals frequently are not required. However, in cases where only a polypeptide coding sequence, or a portion thereof, is inserted, exogenous translational control signals, including an ATG initiation codon is provided for expression of the relevant sequence. The initiation codon is put in the correct reading frame to ensure transcription of the polynucleotide sequence of interest. Exogenous transcriptional elements and initiation codons can be of various origins, both natural and synthetic. The efficiency of expression can be enhanced by the inclusion of enhancers appropriate to the cell system in use (Scharf D et al. (1994) Results Probl Cell Differ 20:125-62; Bittner et al. (1987) Methods in Enzymol 153:516-544).

Expression Hosts

The present invention also relates to host cells which are introduced (transduced, transformed or transfected) with vectors of the invention, and the production of polypeptides of the invention by recombinant techniques. Host cells are genetically engineered (i.e., transduced, transformed or transfected) with a vector, such as an expression vector, of this invention. As described above, the vector can be in the form of a plasmid, a viral particle, a phage, etc. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as COS, CHO, BHK, HEK 293, MCF-7, T-47D, 2329, ZR-75-1, BT-474, SKBR-3, BT-20, MDA-MB-231, CAMA, HCC38, 2336, 2321, 2338, HMEC, MCF-10A, MCF-12A or Bowes melanoma; plant cells, etc.

The engineered host cells can be cultured in conventional nutrient media modified as appropriate for activating promoters, selecting transformants, or amplifying the inserted polynucleotide sequences. The culture conditions, such as temperature, pH and the like, are typically those previously used with the host cell selected for expression, and will be apparent to those skilled in the art and in the references cited herein, including, e.g., Freshney (1994) Culture of Animal Cells, a Manual of Basic Technique, third edition, Wiley-Liss, New York and the references cited therein. Expression products corresponding to the nucleic acids of the invention can also be produced in non-animal cells such as plants, yeast, fungi, bacteria and the like. In addition to Sambrook, Berger and Ausubel, all supra, details regarding cell culture can be found in Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg N.Y.) and Atlas and Parks (eds) The Handbook of Microbiological Media (1993) CRC Press, Boca Raton, Fla.

In bacterial systems, a number of expression vectors can be selected depending upon the use intended for the expressed product. For example, when large quantities of a polypeptide or fragments thereof are needed for the production of antibodies, vectors which direct high-level expression of fusion proteins that are readily purified are favorably employed. Such vectors include, but are not limited to, multifunctional E. coli cloning and expression vectors such as BLUESCRIPT (Stratagene), in which the coding sequence of interest, e.g., sequences comprising SEQ ID NO: 1 through SEQ ID NO: 491, can be ligated into the vector in-frame with sequences for the amino-terminal translation initiating Methionine and the subsequent 7 residues of beta-galactosidase producing a catalytically active beta galactosidase fusion protein; pIN vectors (Van Heeke & Schuster (1989) J Biol Chem 264:5503-5509); pET vectors (Novagen, Madison Wis.); and the like.

Similarly, in the yeast Saccharomyces cerevisiae a number of vectors containing constitutive or inducible promoters such as alpha factor, alcohol oxidase and PGH can be used for production of the desired expression products. For reviews, see Ausubel, supra, and Grant et al., (1987); Methods in Enzymology 153:516-544.

In mammalian host cells, a number of expression systems, such as viral-based systems, can be utilized. In cases where an adenovirus is used as an expression vector, a coding sequence is optionally ligated into an adenovirus transcription/translation complex consisting of the late promoter and tripartite leader sequence. Insertion in a nonessential E1 or E3 region of the viral genome will result in a viable virus capable of expressing the polypeptides of interest in infected host cells (Logan and Shenk (1984) Proc Natl Acad Sci 81:3655-3659). In addition, transcription enhancers, such as the rous sarcoma virus (RSV) enhancer, can be used to increase expression in mammalian host cells.

Transformed or transfected host cells containing the expression vectors described above are also a feature of the invention. The host cell can be an eukaryotic cell, such as a mammalian cell, a yeast cell, or a plant cell, or the host cell can be a prokaryotic cell, such as a bacterial cell. Introduction of the construct into the host cell can be effected by calcium phosphate transfection, DEAE-Dextran mediated transfection, electroporation, or other common techniques (Davis, L., Dibner, M., and Battey, I. (1986) Basic Methods in Molecular Biology).

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing, which cleaves a precursor form into a mature form, of the protein is sometimes important for correct insertion, folding and/or function. Different host cells such as 3T3, COS, CHO, HeLa, BHK, MDCK, 293, WI38, MCF-7, T-47D, 2329, ZR-75-1, BT-474, SKBR-3, BT-20, MDA-MB-231, CAMA, HCC38, 2336, 2321, 2338, HMEC, MCF-10A, MCF-12A, etc. have specific cellular machinery and characteristic mechanisms for such post-translational activities and can be chosen to ensure the correct modification and processing of the introduced, foreign protein.

For long-term, high-yield production of recombinant proteins encoded by or having subsequences encoded by the polynucleotides of the invention, stable expression systems are typically used. For example, cell lines, stably expressing a polypeptide of the invention, are transfected using expression vectors which contain viral origins of replication or endogenous expression elements and a selectable marker gene. Following the introduction of the vector, cells are allowed to grow for 1-2 days in an enriched media before they are switched to selective media. The purpose of the selectable marker is to confer resistance to selection, and its presence allows growth and recovery of cells that successfully express the introduced sequences. For example, resistant clumps of stably transformed cells, e.g., derived from single cell type, can be proliferated using tissue culture techniques appropriate to the cell type.

Host cells transformed with a nucleotide sequence encoding a polypeptide of the invention are optionally cultured under conditions suitable for the expression and recovery of the encoded protein from cell culture. The protein or fragment thereof produced by a recombinant cell can be secreted, membrane-bound, or retained intracellularly, depending on the sequence and/or the vector used.

Polypeptide Production and Recovery

Following transduction of a suitable host cell line or strain and growth of the host cells to an appropriate cell density, the selected promoter is induced by appropriate means (e.g., temperature shift or chemical induction) and cells are cultured for an additional period. The secreted polypeptide product is then recovered from the culture medium. Alternatively, cells can be harvested by centrifugation, disrupted by physical or chemical means, and the resulting crude extract retained for further purification. Eukaryotic or microbial cells employed in expression of proteins can be disrupted by any convenient method, including freeze-thaw cycling, sonication, mechanical disruption, or use of cell lysing agents, or other methods, which are well know to those skilled in the art.

Expressed polypeptides can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the mature protein. Finally, high performance liquid chromatography (HPLC) can be employed in the final purification steps. In addition to the references noted supra, a variety of purification methods are well known in the art, including, e.g., those set forth in Sandana (1997) Bioseparation of Proteins, Academic Press, Inc.; and Bollag et al. (1996) Protein Methods, 2^{nd} Edition Wiley-Liss, New York; Walker (1996) The Protein Protocols Handbook Humana Press, New Jersey, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, New York; Janson and Ryden (1998) Protein Purification: Principles, High Resolution Methods and Applications, Second Edition Wiley-VCH, New York; and Walker (1998) Protein Protocols on CD-ROM Humana Press, New Jersey.

Alternatively, cell-free transcription/translation systems can be employed to produce polypeptides comprising an amino acid sequence or subsequence of, e.g., SEQ ID NO: 492, or encoded by the polynucleotide sequences of the invention. A number of suitable in vitro transcription and translation systems are commercially available. A general guide to in vitro transcription and translation protocols is found in Tymms (1995) In vitro Transcription and Translation Protocols: Methods in Molecular Biology Volume 37, Garland Publishing, New York.

In addition, the polypeptides, or subsequences thereof, e.g., subsequences comprising antigenic peptides, can be produced manually or by using an automated system, by direct peptide synthesis using solid-phase techniques (see, Stewart et al. (1969) Solid-Phase Peptide Synthesis, W H Freeman Co, San Francisco; Merrifield J (1963) J. Am. Chem. Soc. 85:2149-2154). Exemplary automated systems include the Applied Biosystems 431A Peptide Synthesizer (Perkin Elmer, Foster City, Calif.). If desired, subsequences can be chemically synthesized separately, and combined using chemical methods to provide full-length polypeptides.

### Conservatively Modified Variations

The isolated or recombinant polypeptides of the present invention include conservatively modified variations of polypeptides comprising subsequences, e.g., of SEQ ID NO: 492, or encoded by a polynucleotide of the invention, e.g., SEQ ID NO: 1-SEQ ID NO: 491. Such conservatively modified variations comprise substitutions, additions or deletions, which alter, add or delete a single amino acid or a small percentage of amino acids (typically less than about 5%, more typically less than about 4%, about 2%, or about 1%). Typically, substitutions of amino acids are conservative substitutions according to the six substitution groups set forth in Table 1 (supra).

For example, conservatively substituted variation of the polypeptide identified herein as SEQ ID NO: 492 will contain “conservative substitutions”, according to the six groups defined above, in up to 43 residues (i.e., 5% of the amino acids) in the 862 amino acid polypeptide.

For example, if four conservative substitutions were localized in the region corresponding to amino acids 3-27 of SEQ ID NO: 492, examples of conservatively substituted variations of this region,

GQWFYEAKAKRHRDKIHGADIIRAS include:

GQWYYEARAKRHRDKVHGAEIIRAS and

GNWFFEAKAKRHRDKIHGADVIKAS and the like, in accordance with the conservative substitutions listed in Table 1 (in the above example, conservative substitutions are underlined). Listing of a protein sequence herein, in conjunction with the above substitution table, provides an express listing of all conservatively substituted proteins.

The addition of sequences that do not alter the encoded activity of a nucleic acid molecule, such as the addition of a non-functional sequence, is a conservative variation of the basic nucleic acid. For example, the polypeptides of the invention, including conservatively substituted sequences, can be present as part of larger polypeptide sequences such as occur upon the addition of one or more domains for purification of the protein (e.g., poly his segments, FLAG tag segments, green fluorescent protein (GGP) etc.), e.g., where the additional functional domains have little or no effect on the activity of the protein, or where the additional domains can be removed by post synthesis processing steps such as by treatment with a protease.

Modified Amino Acids

Expressed polypeptides of the invention can contain one or more modified amino acid. The presence of modified amino acids can be advantageous in, for example, (a) increasing polypeptide serum half-life, (b) reducing polypeptide antigenicity, (c) increasing polypeptide storage stability. Amino acid(s) are modified, for example, co-translationally or post-translationally during recombinant production (e.g., N-linked glycosylation at N-X-S/T motifs during expression in mammalian cells) or modified by synthetic means (e.g., via PEGylation).

Non-limiting examples of a modified amino acid include a glycosylated amino acid, a sulfated amino acid, a prenlyated (e.g., farnesylated, geranylgeranylated) amino acid, an acetylated amino acid, an acylated amino acid, a PEG-ylated amino acid, a biotinylated amino acid, a carboxylated amino acid, a phosphorylated amino acid, and the like, as well as amino acids modified by conjugation to, e.g., lipid moieties or other organic derivatizing agents. References adequate to guide one of skill in the modification of amino acids are replete throughout the literature. Example protocols are found in Walker (1998) Protein Protocols on CD-ROM Human Press, Towata, N.J.

### Antibodies

The polypeptides of the invention can be used to produce antibodies specific for the polypeptide of, e.g., SEQ ID NO: 492 and/or polypeptides encoded by the polynucleotides of the invention, e.g., SEQ ID NO: 1-SEQ ID NO: 491, and conservative variants thereof. Antibodies specific for the above mentioned polypeptides are useful, e.g., for diagnostic and therapeutic purposes, e.g., related to the activity, distribution, and expression of target polypeptides. For example, antibodies that block receptor binding, are useful for certain therapeutic applications.

Antibodies specific for the polypeptides of the invention can be generated by methods well known in the art. Such antibodies can include, but are not limited to, polyclonal, monoclonal, chimeric, humanized, single chain, Fab fragments and fragments produced by an Fab expression library.

Polypeptides do not require biological activity for antibody production. However, the polypeptide or oligopeptide must be antigenic. Peptides used to induce specific antibodies typically have an amino acid sequence of at least about 4 amino acids, and often at least about 5 or about 10 amino acids. Short stretches of a polypeptide can be fused with another protein, such as keyhole limpet hemocyanin, and antibody produced against the chimeric molecule.

Numerous methods for producing polyclonal and monoclonal antibodies are known to those of skill in the art, and can be adapted to produce antibodies specific for the polypeptides of the invention, e.g., SEQ ID NO: 492 and/or encoded by SEQ ID NO: 1-SEQ ID NO: 491. See, e.g., Coligan (1991) Current Protocols in Immunology Wiley/Greene, New York; Paul (Ed.) (1998) Fundamental Immunology, Fourth Edition, Lippinocott-Raven, Lippincott Williams & Wilkins; Harlow and Lane (1989) Antibodies: A Laboratory Manual Cold Spring Harbor Press, New York; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256: 495-497. Other suitable techniques for antibody preparation include selection of libraries of recombinant antibodies in phage or similar vectors. See, Huse et al. (1989) Science 246: 1275-1281; and Ward, et al. (1989) Nature 341: 544-546. Specific monoclonal and polyclonal antibodies and antisera will usually bind with a K_{D} of, e.g., at least about 0.1 μM, at least about 0.01 μM or better, and, typically and at least about 0.001 μM or better.

For certain therapeutic applications, humanized antibodies are desirable. Detailed methods for preparation of chimeric (humanized) antibodies can be found in U.S. Pat. No. 5,482,856. Additional details on humanization and other antibody production and engineering techniques can be found in Borrebaeck (ed) (1995) Antibody Engineering, 2^{nd} Edition Freeman and Company, New York (Borrebaeck); McCafferty et al. (1996) Antibody Engineering, A Practical Approach IRL at Oxford Press, Oxford, England (McCafferty), and Paul (1995) Antibody Engineering Protocols Humana Press, Towata, N.J. (Paul). Additional details regarding specific procedures can be found, e.g., in Ostberg et al. (1983), Hybridoma 2: 361-367, Ostberg, U.S. Pat. No. 4,634,664, and Engelman et al., U.S. Pat. No. 4,634,666.

Defining Polypeptides by Immunoreactivity

The polypeptides of the invention encoded by the sequence listing herein, as well as novel variants derived therefrom, which are also encompassed within the present invention, provide a variety of structural features which can be recognized, e.g., in immunological assays. The generation of antisera which specifically binds the polypeptides of the invention, as well as the polypeptides which are bound by such antisera, are a feature of the invention.

The invention includes polypeptides that specifically bind to or that are specifically immunoreactive with an antibody or antisera generated against an immunogen comprising an amino acid sequence, e.g., of SEQ ID NO: 492, and/or encoded by one or more polynucleotide sequences of the invention, e.g., selected from one or more of SEQ ID NO: 1 to SEQ ID NO: 491. To eliminate cross-reactivity with non-related polypeptides, the antibody or antisera can be subtracted with unrelated polypeptides or proteins.

In one typical format, the immunoassay uses a polyclonal antiserum which was raised against one or more polypeptide comprising one or more of the sequences corresponding to one or more polypeptides of the invention, such as SEQ ID NO: 492 and/or encoded by one or more of polynucleotide sequences of the invention, e.g., selected from SEQ ID NO: 1-SEQ ID NO: 491, or a subsequence thereof (e.g., a substantial subsequence including at least about 30% of the full length sequence provided). Such an antigenic peptide or polypeptide is referred to as an “immunogenic polypeptide.” The resulting antisera is optionally selected to have low cross-reactivity against unrelated polypeptides, e.g., BSA, and any such cross-reactivity can be removed by immunoabsorbtion with one or more of the unrelated polypeptides, or protein preparations, prior to use of the polyclonal antiserum in the immunoassay.

In order to produce antisera for use in an immunoassay, one or more of the immunogenic polypeptides is produced and purified as described herein. For example, recombinant protein can be produced in a mammalian cell line. An inbred strain of mice (used in this assay because results are more reproducible due to the virtual genetic identity of the mice) is immunized with the immunogenic protein(s) in combination with a standard adjuvant, such as Freund's adjuvant, and a standard mouse immunization protocol (see, Harlow and Lane (1989), supra, for a standard description of antibody generation, immunoassay formats and conditions that can be used to determine specific immunoreactivity). Alternatively, one or more synthetic or recombinant polypeptide derived from the sequences disclosed herein is conjugated to a carrier protein and used as an immunogen.

Polyclonal sera are collected and titered against the immunogenic polypeptide in an immunoassay, for example, a solid phase immunoassay with one or more of the immunogenic proteins immobilized on a solid support. Polyclonal antisera with a titer of 10⁶ or greater are selected, pooled and subtracted with the control unrelated polypeptides to produce subtracted pooled titered polyclonal antisera.

If desired, the subtracted pooled titered polyclonal antisera are tested for cross reactivity against any unrelated polypeptides. Discriminatory binding conditions are determined for the subtracted titered polyclonal antisera which result in at least about a 5-10 fold higher signal to noise ratio for binding of the titered polyclonal antisera to the immunogenic polypeptide of interest as compared to binding to the unrelated polypeptide. That is, the stringency of the binding reaction is adjusted by the addition of non-specific competitors such as albumin or non-fat dry milk, or by adjusting salt conditions, temperature, or the like. These binding conditions are used in subsequent assays for determining whether a test polypeptide is specifically bound by the pooled subtracted polyclonal antisera. In particular, test polypeptides which show at least a 2-5× and preferably 10× or higher signal to noise ratio than the control polypeptides under discriminatory binding conditions, and at least about a ½ signal to noise ratio as compared to the immunogenic polypeptide(s) (and typically 90% or more of the signal to noise ratio shown for the immunogenic peptide), shares substantial structural similarity with the immunogenic polypeptide as compared to unrelated polypeptides, and is, therefore, a polypeptide of the invention.

Such methods are also useful for detecting an unknown test protein or polypeptide, which is also specifically bound by the antisera under conditions as described above. In one format, the immunogenic polypeptide(s) are immobilized to a solid support which is exposed to the subtracted pooled antisera. Test proteins are added to the assay to compete for binding to the pooled subtracted antisera. The ability of the test protein(s) to compete for binding to the pooled subtracted antisera as compared to the immobilized protein(s) is compared to the ability of the immunogenic polypeptide(s) added to the assay to compete for binding (the immunogenic polypeptides compete effectively with the immobilized immunogenic polypeptides for binding to the pooled antisera). The percent cross-reactivity for the test proteins is calculated, using standard calculations.

In a parallel assay, the ability of the control proteins to compete for binding to the pooled subtracted antisera is determined as compared to the ability of the immunogenic polypeptide(s) to compete for binding to the antisera. Again, the percent cross-reactivity for the control polypeptides is calculated, using standard calculations. Where the percent cross-reactivity is at least 5-10× as high for the test polypeptides, the test polypeptides are said to specifically bind the pooled subtracted antisera.

In general, the immunoabsorbed and pooled antisera can be used in a competitive binding immunoassay as described herein to compare any test polypeptide to the immunogenic polypeptide(s). In order to make this comparison, the two polypeptides are each assayed at a wide range of concentrations and the amount of each polypeptide required to inhibit 50% of the binding of the subtracted antisera to the immobilized protein is determined using standard techniques. If the amount of the test polypeptide required is less than twice the amount of the immunogenic polypeptide that is required, then the test polypeptide is said to specifically bind to an antibody generated to the immunogenic protein, provided the amount is at least about 5-10× as high as for a control polypeptide.

As a final determination of specificity, the pooled antisera is optionally fully immunosorbed with the immunogenic polypeptide(s) (rather than the control polypeptides) until little or no binding of the resulting immunogenic polypeptide subtracted pooled antisera to the immunogenic polypeptide(s) used in the immunosorbtion is detectable. This fully immunosorbed antisera is then tested for reactivity with the test polypeptide. If little or no reactivity is observed (i.e., no more than 2× the signal to noise ratio observed for binding of the fully immunosorbed antisera to the immunogenic polypeptide), then the test polypeptide is specifically bound by the antisera elicited by the immunogenic protein.

### Predicting Breast Cancer

The probes and marker sets of the invention are favorably employed in methods for predicting breast cancer in a subject, such as a patient undergoing medical evaluation for risk of breast cancer. Nucleic acids of a marker set or individual probes including one or more polynucleotides of the invention, as described, e.g., in the section entitled “Probes,” are hybridized, e.g., as an array, to a DNA or RNA sample from a subject cell or tissue sample. Upon hybridization of the sample to at least a subset of the probes, a signal is detected corresponding to at least one polymorphic nucleic acid or to expression or activity of an expression product correlatable to breast cancer. When expression is detected, the evaluation can be made on a qualitative basis, that is, detecting whether or not an expression product (or multiple expression products) are expressed in a subject cell or tissue sample. Alternatively, the evaluation can be quantitative, that is, determining whether levels are adequate to provide the desired characteristic.

The subject sample is usually selected for ease of acquisition and to minimize invasiveness of the collection procedure to the subject. Thus, in the context of human subjects, breast tissue samples can be obtained by well-known procedures. In the case of certain experimental applications, e.g., using animal models, breast tissue samples can also be obtained by well-known procedures.

For example, a marker set including a plurality of the polynucleotides of the invention, can be hybridized individually, or as an array, to an RNA or cDNA sample produced, e.g., by a reverse transcription-polymerase chain reaction (RT-PCR), from a subject RNA sample. Typically, prior to hybridization of the probes or array to a subject or “test” sample, the probe or array is validated and/or calibrated by comparing samples obtained from classes of subjects known to differ with respect to their risk or stage of breast cancer. For example, subjects shown, e.g., by unresponsiveness to hormone treatment, to be at enhanced risk of metastatic breast cancer are compared to breast cancer subjects that show response to hormone treatment.

Alternatively, a marker set including a plurality of antibodies, or other binding proteins, specific polypeptide or peptide, e.g., of SEQ ID NO: 492, and/or encoded by a polynucleotide of the invention, e.g., selected from SEQ ID NO: 1-SEQ ID NO: 491, are employed as individual probes or marker sets to evaluate expression of proteins in a cell or tissue sample. In this case, rather than, or in addition to, preparing RNA from a sample, proteins are recovered and exposed to the probe or marker set of antibodies, in liquid phase or with either the target of antibody immobilized on a solid substrate, such as a solid phase array.

Patterns of expression correlatable to breast cancer are detected by hybridization to one or more probes. In some embodiments, a single probe with a high predictive value is favored, e.g., for ease of handling and cost containment. In other embodiments multiple probes, e.g., the entire marker set, or a type of marker set, e.g., identifying a breast cancer cell (either ER+ or ER−) compared to normal mammary epithelia (e.g., SEQ ID NO: 1-492) or identifying a type of breast cancer cell, e.g., ER+ or ER− (e.g., SEQ ID NO: 1-286, 492), are typically used, e.g., to increase sensitivity or diagnostic or prognostic value. Optimal probes and marker sets are readily ascertained on an empirical basis.

Alternatively, an oligonucleotide or polynucleotide probe can detect sequence polymorphisms, rather than expression differences, between subjects in different breast cancer risk classes. Polymorphisms at a nucleotide level can correspond either directly or indirectly to the gene of interest underlying breast cancer susceptibility, and can be detected in any of several ways, for example, as restriction fragment length polymorphisms, by allele specific hybridization, as amplification length polymorphisms, and the like.

For example, oligonucleotide probes including conservative variants of a polynucleotide sequences are selected that correspond to polymorphic variations in a target sequence. For example, a probe pair incorporating a single variant nucleotide can be designed to hybridize under allele specific hybridization conditions to allelic target sequences in which one allele is indicative of breast cancer and the other allele indicates, e.g., a relatively reduced susceptibility. For example, probes sequences are selected from among SEQ ID NO: 1 through SEQ ID NO: 491 (or other polynucleotides of the invention) and variants thereof. In some instances, for example, where the cDNA or chromosomal segment has been sequenced and a particular nucleotide polymorphism is associated with breast cancer susceptibility, the probes are chosen to detect the nucleotide polymorphism, e.g., by allele specific hybridization.

### Modulating Breast Cancer in a Cell or Tissue

The invention also provides experimental and therapeutic methods for modulating breast cancer in vitro and in vivo. Tissue culture and animal models useful for elucidating the molecular mechanisms underlying breast cancer as well as for screening and evaluating potential therapeutic targets are produced by modulating expression or activity of polypeptides (e.g., such as SEQ ID NO: 492 or encoded by the polynucleotides of invention, e.g., SEQ ID NO: 1-SEQ ID NO: 491).

For example, mammalian cells in culture are transfected with a nucleic acid, e.g., comprising a polynucleotide selected from SEQ ID NO: 1 through SEQ ID NO: 491, to produce cells that express a polypeptide involved in the transformation of a normal mammary epithelia cell to a breast cancer cell or from an ER+ breast cancer cell to an ER− breast cancer cell or involved in the type of breast cancer cell, e.g., ER+ breast cancer cell verses an ER− breast cancer cell. It will be understood, that where exogenous polynucleotide sequences are introduced into cells, tissues or organisms, that the polynucleotide sequences can be selected from among SEQ ID NO: 1-491 (or, e.g., among SEQ ID NO: 1-286 for distinguishing the type of breast cancer cell, ER+ verses ER−), sequences that hybridize under stringent conditions to any one of SEQ ID NO: 1-SEQ ID NO: 491, sequences that are at least about 70% identical to any one of SEQ ID NO: 1-SEQ ID NO: 491, sequences that encode a polypeptide or peptide comprising a subsequence encoded by any one of SEQ ID NO: 1-SEQ ID NO: 491, sequences that are physically linked in the human genome to any one of SEQ ID NO: 1-SEQ ID NO: 491, sequences complementary to any such sequences, or subsequences thereof.

In some cases, it is preferable to link the polynucleotide sequence of interest to the regulatory sequences with which it is typically associated in vivo in nature. Alternatively, in cases where constitutive expression at levels that are in excess of those found in nature is desired, exogenous promoters and enhancers can be employed, as described in detail in the section entitled “Vectors, Promoters and Expression Systems.”

Expression and/or activity of the gene or polypeptide can also be modulated in a negative manner, that is, suppressed. For example, knock out mutations can be produced by homologous recombination of an exogenous gene homologue, e.g., bearing stop codon, and/or insertion of, e.g., a selectable marker, that disrupts production of an intact transcript. Alternatively, vectors incorporating the sequence of interest in the antisense orientation can be introduced to suppress translation at a post-transcriptional level.

Alternatively, cell lines that express polypeptides comprising a subsequence encoded by a polynucleotide of the invention into which vectors have been transduced that randomly activate expression of associated endogenous sequences upon integration can be isolated. Such vectors have been described, e.g., by Harrington et al. (2001) “Creation of genome-wide protein expression libraries using random activation of gene expression.” Nature Biotechnology 19: 440-445, which is incorporated herein by reference. Typically, the vector is constructed with a strong exogenous promoter linked to an exon and an unpaired splice donor site. Upon integration into the genome, splicing with a proximal splice-acceptor site occurs, activating expression of a chimeric transcript encoding at least a portion of the endogenous gene. Cells expressing a polypeptide of interest can be selected by well known methods, including those based on phenotypic screening methods, antibody or receptor binding, RNA analytical methods, e.g., RT-PCR, northern analysis, MPSS, and the like. Typically, the screening is performed in a high-throughput format.

In certain embodiments, modulation of expression or activity of the polypeptide encoded by the transfected polynucleotide contributes to a detectable breast cancer phenotype. Thus, in one embodiment, modulation of expression or activity of a polynucleotide corresponding to the breast cancer locus is achieved by inducing or suppressing expression of the polynucleotide or by introducing a mutation that results in an increase or decrease in the activity of the encoded polypeptide.

The above-described methods for producing cell culture model systems can be adapted for use in the screening of therapeutic interventions, e.g., aimed at identifying breast cancer and the type of breast cancer. For example, it is desirable to select promoters and enhancers that are modulated in response to hormones, e.g., estrogen and/or progesterone, or other molecules, such as pharmaceutical agents.

Following treatment with hormones, e.g., estrogen and/or progrestrone, other molecules, such as pharmaceutical agents, e.g., tamoxifen, altered expression or activity can be detected at the RNA or protein level. Detection of altered levels of RNA is most conveniently accomplished by such methods as RT-PCR, MPSS, or northern analysis. Protein expression is conveniently monitored using, e.g., antibody based detection methods, such as ELISA's, immunoprecipitations, or immunohistochemical methods including Western analysis. In each of these protein expression procedures, the sample including the expressed protein of interest is reacted with an antibody (e.g., monoclonal antibody) or antiserum specific for the protein of interest. Methods for generating specific antibodies are well known and further details are provided above in the section entitled “Antibodies.”

The cell culture models can be used to identify pharmaceutical agents capable of favorably regulating the expression or activity of a polypeptide of interest, e.g., a polypeptide comprising an amino acid sequence or subsequence of SEQ ID NO: 492 and/or encoded by a polynucleotide of the invention, in a cell culture system as described above. Most typically, this involves exposing the cells to a chemical or biological composition, e.g., a small organic molecule, or biological macromolecule such as a protein, e.g., an antibody, binding protein, or macromolecular cofactor. Following exposure to the one or more compositions, for example, members of a chemical or biological composition library, such as a combinatorial chemical library, a library of peptide or polypeptide products expressed from a library of nucleic acids, an antibody (or other polypeptide) display library such as a phage display library, etc., modulation of the polypeptide of interest is detected. As discussed above, modulation of the polypeptide can be detected as an alteration in expression at the level of transcription or translation, or as an alteration in the activity of the encoded protein or polypeptide. In some instances, it is desirable to monitor expression or activity of multiple expression products in the same cell, or cell line. The monitored expression products, can be exogenous, i.e., introduced as described above, or endogenous, such as transcripts or polypeptides whose expression or activity is dependent on the amount or activity of a polypeptide of interest.

In cases where the expression or activity of multiple products are of interest, or where the effect of a plurality of different compounds on the expression or activity of one or more expression products, e.g., screening for pharmaceutical agents as described above, the monitoring assay is conveniently performed in an array. For example, cells can be arrayed by aliquoting into the wells of a multiwell plate, e.g., a 96, 384, 1536, or other convenient format selected according to available equipment. The arrayed cells can exposed to members of a composition library, and the cells sampled and monitored by, e.g., FACS, immunohistochemisty, ELISA, etc. Alternatively, nucleic acids or proteins can be prepared from the arrayed cells, in a manual, semi-automatic or automated procedure, and the products arranged in a liquid or solid phase array for evaluation. Additional details regarding arrays are provided above in the section entitled “Marker Sets.” Alternative high throughput processing methods, such as microfluidic devices, are also available, and can favorably be employed in the context of monitoring modulation of expression products.

Typically, when processing and evaluating large numbers of samples, e.g., in a high throughput assay, data relating to expression or activity is recorded in a database, typically the database includes character strings representing the data recorded on a computer or in a computer readable medium.

In addition to tissue culture systems, transgenic animals, most typically non-human mammals, can be produced which have integrated one or more of the polynucleotide sequences of the invention, e.g., comprising a subsequence selected from selected from SEQ ID NO: 1 to SEQ ID NO: 491. In this context, commonly used experimental animals include, e.g., mouse, rat, rabbit (e.g., New Zealand White), dog, pig, sheep, or a non-human primate. In some cases the animal of choice has a naturally occurring or introduced mutation in a gene encoding a protein involved in breast cancer.

Such transgenic animal models are useful, in addition to the cultured cells discussed above, for the evaluation of pharmaceutical agents suitable for the modulation of breast cancer. Transgenic animal models, e.g., expressing a polypeptide comprising a sequence or subsequence of SEQ ID NO: 492 or encoded by a polynucleotide sequence of the invention, e.g., selected from SEQ ID NO: 1-491, are also suitable for evaluating breast cancer. For example, following administration of a hormone, e.g., estrogen and/or progestrone, or other molecules, such as pharmaceutical agents, e.g., tamoxifen, to a transgenic animal expressing a polypeptide of the invention, gene expression is monitored. Monitoring can involve detecting altered expression or activity of an expression product encoded by one or more polynucleotide of the invention as discussed above. Alternatively, standard clinical laboratory methods for detecting and evaluating breast cancer gene expression profiles in the serum can be utilized. Such assays can also be adapted to evaluate cancer cell quantity and composition in breast tissues and/or other organs, e.g., lymph nodes.

### Administration in Patients

In one aspect, the present invention provides for the administration of one or more of the nucleic acids herein, e.g., for gene therapy and/or for the administration of a protein herein as a prophylactic or therapeutic agent to a subject, including, e.g., a mammal, including, e.g., a human, primate, mouse, pig, cow, goat, rabbit, rat, guinea pig, hamster, horse, and/or sheep. In addition, modulators of expression of genes encoding the nucleic acids or proteins herein and/or activity modulators of the proteins herein can be administered to regulate the transformation of normal mammary epithelia to a breast cancer cell or the transformation of ER+ breast cancer cells to ER− breast cancer cells.

Whether the therapeutic agent is a nucleic acid, a protein or a modulator of an activity of a nucleic acid or protein, administration is by any of the routes normally used for introducing a molecule into ultimate contact with blood or tissue cells. Suitable methods of administering compositions in the context of the present invention to a patient are available, and, although more than one route can be used to administer a particular composition, a particular route can provide a more immediate and more effective reaction than another route.

The invention also includes compositions comprising any nucleic acid or any isolated or recombinant polypeptide described above and an excipient, e.g., a pharmaceutically acceptable excipient. Transgenic animals, which include any nucleic acid or polypeptide above, e.g., produced by introduction of the vector, are also a feature of the invention. Methods for treating breast cancer by administering to a patient an effective amount of at least one expression vector and/or an effective amount of at least one isolated or recombinant polypeptide described above are also included in the present invention.

Pharmaceutically acceptable excipents or carriers are determined in part by the particular composition being administered, as well as by the particular method used to administer the composition. Accordingly, there are a wide variety of suitable formulations of pharmaceutical compositions of the present invention.

Formulations suitable for parenteral administration, such as, for example, by intraarticular (in the joints), intravenous, intramuscular, intradermal, subdermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Parenteral administration and intravenous administration are one class of preferred methods of administration. Formulations can be presented in unit-dose or multi-dose sealed containers, such as ampules and vials.

Injection solutions and suspensions can be prepared from sterile powders, granules, and tablets. Cells transduced by expression vectors or gene therapy vectors (e.g., in the context of ex vivo gene therapy) can also be administered intravenously or parenterally as described above.

Formulations suitable for oral administration can consist of (a) liquid solutions, such as an effective amount of the packaged nucleic acid suspended in diluents, such as water, saline, buffered saline, ethanol, glycerol, dextrose, PEG 400 and combinations thereof; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art.

The materials, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules that consist of a combination of materials with a base, including, for example, liquid triglycerides, polyethylene glycols, and paraffin hydrocarbons.

The dose administered to a patient, in the context of the present invention should be sufficient to affect a beneficial therapeutic response in the patient over time. The dose will be determined by the efficacy of the particular composition employed and the condition of the patient, as well as the body weight or surface area of the patient to be treated. The size of the dose also will be determined by the existence, nature, and extent of any adverse side-effects that accompany the administration of a particular composition (e.g., gene therapy vector, transduced cell type, protein or activity modulator) in a particular patient.

In determining an effective amount to be administered in the treatment or prophylaxis of breast cancer or an associated condition, the physician evaluates tumor size and type, vector toxicities, progression of disease, and, e.g., production of antibodies to the therapeutic composition.

For example, in one aspect, the dose equivalent of a naked nucleic acid encoding a nucleic acid herein is from about 0.1 μg to 1 mg for a typical 70 kilogram patient, and doses of vectors which include a gene therapy or expression vector, such as a retroviral particle, are calculated to yield an approximately equivalent amount of a nucleic acid.

In the practice of this invention, compositions can be administered, for example, by intravenous infusion, orally, topically, intraperitoneally, intravesically or intrathecally. The method of administration will often be local, oral, rectal or intravenous, but materials can also be applied in a suitable vehicle for the topical treatment of related conditions. The agents of this invention can supplement treatment of breast cancer related conditions by any known conventional therapy, including pain medications, biologic response modifiers and the like.

For administration, compositions of the present invention can be administered at a rate determined by the LD-50 of composition and the side-effects of the composition at various concentrations, as applied to the mass and overall health of the patient. Administration can be accomplished via single or divided doses.

For ex-vivo therapy, transduced cells are prepared for reinfusion according to established methods. See, Abrahamsen et al. (1991) J. Clin. Apheresis 6:48-53; Carter et al. (1988) J. Clin. Arpheresis 4:113-117; Aebersold et al. (1988), J. Immunol. Methods 112: 1-7; Muul et al. (1987) J. Immunol. Methods 101:171-181 and Carter et al. (1987) Transfusion 27:362-365. After a period of about 2-4 weeks in culture, the cells should number between 1×10⁸ and 1×10¹². In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent.

In one embodiment, in ex vivo methods, one or more cells, or a population of the subject's cells of interest, e.g., tumor cells, tumor tissue sample, blood cells, cells of the breast, are obtained or removed from the subject and contacted with an amount of a molecule of the invention, e.g., nucleic acids or subsequences thereof or isolated or recombinant polypeptides or subsequences thereof or antibodies, that is effective in prophylactically or therapeutically treating breast cancer. The contacted cells are then returned or delivered to the subject to the site from which they were obtained or to another site (e.g., including those defined above) of interest in the subject to be treated. Contacted cells can also be grafted onto a tissue or system site (including all described above) of interest in the subject using standard and well-known grafting techniques or, e.g., delivered to the blood or lymph system using standard delivery or transfusion techniques. In another embodiment, a construct comprising a molecule, e.g., a nucleic acid sequence of the invention, e.g., SEQ ID NO: 1 to SEQ ID NO: 491, that encodes a biologically active peptide that is effective in prophylactically or therapeutically treating breast cancer, is introduced into the one or more cells of interest or a population of cells of interest of the subject. A sufficient amount of the construct and a controlling promotor is used such that uptake of the construct (and promoter) into the cell(s) occurs and sufficient expression of the biologically active peptide produces an amount of the biologically active molecule effective to prophylactically or therapeutically treat breast cancer. Expression of the target nucleic acid can either be induced or occur naturally and a sufficient amount of the molecule is expressed and effective to treat the disease or condition at the site or tissue system.

In another embodiment, the invention provides in vivo methods in which one or more cells or a population of the subject's cells of interest are contacted directly or indirectly with an amount of a molecule(s) of the invention effective in prophylactically or therapeutically treating breast cancer. In direct contact/administration formats, the molecule(s) is typically administered or transferred directly to the cells to be treated or to the tissue site of interest (e.g., breast tissue) by any of a variety of formats, which include injection, e.g., by a needle and/or syringe, vaccine, gene gun delivery, or pushing into a breast tissue. The molecule(s) can be delivered as described above, or placed within a cavity of the body (including, e.g., during surgery).

In in vivo indirect contact/administration formats, the molecule(s) is administered or transferred indirectly to the cells to be treated or to the tissue site of interest, such as, e.g., breast tissue, lymphatic system, or blood cell system, etc, by contacting or administering the molecule(s) of the invention directly to one or more cells or population of cells from which treatment can be facilitated. For example, breast tumor cells within the body of the subject can be treated by contacting cells of the blood or lymphatic system or breast tissue with a sufficient amount of the molecule such that delivery of the molecule to the site of interest (e.g., breast tissue, or mammary cells or blood or lymphatic system within the body) occurs and effective prophylactic or therapeutic treatment results. Such contact, administration, or transfer is typically made by using one or more of the routes or modes of administration described above.

In one embodiment, the invention provides in vivo methods. Typically, one or more cells of interest or a population of subject's cells (e.g., including those cells and cell(s) systems and subjects described above) are transformed in the body of the subject by contacting the cell(s) or population of cells with (or administering or transferring to the cell(s) or population of cells using one or more of the routes or modes of administration described above) a polynucleotide construct comprising a nucleic acid sequence of the invention that encodes a biologically active molecule of interest (e.g., a polynucleotide of the invention) that is effective in prophylactically or therapeutically treating the breast cancer or other condition. Expression of the nucleic acid can be induced or occur naturally such that an amount of the encoded polypeptide expressed is sufficient and effective to treat breast cancer. The polynucleotide construct can include a promoter sequence (e.g., CMV promoter sequence) and optionally, one or more additional nucleotide sequences of the invention, adjuvant, or co-stimulatory molecule, or other polypeptide of interest.

A variety of viral vectors suitable for in vivo transduction and expression in an organism are known. Such vectors include retroviral vectors (see, Miller (1992) Curr. Top. Microbiol. Immunol 158:1-24; Salmons and Gunzburg (1993) Human Gene Therapy 4:129-141; Miller et al. (1994) Methods in Enzymology 217: 581-599), adeno-associated vectors (reviewed in Carter (1992) Curr. Opinion Biotech. 3: 533-539; Muzcyzka (1992) Curr. Top. Microbiol. Immunol. 158: 97-129) and other viral vectors (as generally described in, e.g., Jolly (1994) Cancer Gene Therapy 1:51-64; Latchman (1994) Molec. Biotechnol. 2:179-195; and Johanning et al. (1995) Nucl. Acids Res. 23:1495-1501).

If a patient undergoing infusion of a therapeutic composition develops fevers, chills, or muscle aches, he/she receives the appropriate dose of aspirin, ibuprofen or acetaminophen. Patients who experience reactions to the infusion such as fever, muscle aches, and chills are premedicated 30 minutes prior to the future infusions with either aspirin, acetaminophen, or diphenhydramine. Meperidine is used for more severe chills and muscle aches that do not quickly respond to antipyretics and antihistamines. Cell infusion is slowed or discontinued depending upon the severity of the reaction.

In general, gene therapy provides methods for combating diseases, e.g., breast cancer, and some forms of congenital defects such as enzyme deficiencies. Various textbooks describe gene therapy protocols which can be used with the present invention by introducing nucleic acids, e.g., one or more of SEQ ID NO: 1 to SEQ ID NO: 491, into patient. One example is Robbins (1996) Gene Therapy Protocols, Humana Press, New Jersey, and Joyner (1993) Gene Targeting: A Practical Approach, IRL Press, Oxford, England.

In addition to the references cited above, several approaches for introducing nucleic acids into cells in vivo, ex vivo and in vitro are also described below along with the references cited within. These include liposome based gene delivery (Debs and Zhu (1993) WO 93/24640 and U.S. Pat. No. 5,641,662; Mannino and Gould-Fogerite (1988) BioTechniques 6(7): 682-691; Rose, U.S. Pat. No. 5,279,833; Brigham (1991) WO 91/06309; and Feigner et al. (1987) Proc. Natl. Acad. Sci. USA 84: 7413-7414); Brigham et al. (1989) Am. J. Med Sci., 298:278-281; Nabel et al. (1990) Science, 249:1285-1288; Hazinski et al. (1991) Am. J. Resp. Cell Molec. Biol., 4:206-209; and Wang and Huang (1987) Proc. Natl. Acad. Sci USA, 84:7851-7855); adenoviral vector mediated gene delivery, e.g., to treat cancer (see, e.g., Chen et al. (1994) Proc. Natl. Acad. Sci. USA 91: 3054-3057; Tong et al. (1996) Gynecol. Oncol. 61: 175-179; Clayman et al. (1995) Cancer Res. 5: 1-6; O'Malley et al. (1995) Cancer Res. 55: 1080-1085; Hwang et al. (1995) Am. J. Respir. Cell Mol. Biol. 13: 7-16; Haddada et al. (1995) Curr. Top. Microbiol. Immunol. 199 (Pt. 3): 297-306; Addison et al. (1995) Proc. Nat'l. Acad. Sci USA 92: 8522-8526; Colak et al. (1995) Brain Res 691: 76-82; Crystal (1995) Science 270: 404-410; Elshami et al. (1996) Human Gene Ther. 7: 141-148; Vincent et al. (1996) J. Neurosurg. 85: 648-654). Other delivery systems include replication-defective retroviral vectors harboring therapeutic polynucleotide sequence as part of the retroviral genome, particularly with regard to simple MuLV vectors (Miller et al. (1990) Mol. Cell. Biol. 10:4239 (1990); Kolberg (1992) J. NIH Res. 4:43, and Cornetta et al. (1991) Hum. Gene Ther. 2:215), nucleic acid transport coupled to ligand-specific, cation-based transport systems (Wu and Wu (1988) J. Biol. Chem., 263:14621-14624) and naked DNA expression vectors (Nabel et al. (1990), supra); Wolff et al. (1990) Science, 247:1465-1468). In general, these approaches can be adapted to the invention by incorporating nucleic acids, e.g., one or more of SEQ ID NO: 1 to SEQ ID NO: 491 herein, into the appropriate vectors.

In addition to expression of the nucleic acids of the invention as gene replacement nucleic acids, the nucleic acids are also useful for sense and anti-sense suppression of expression, e.g., to down-regulate expression of a nucleic acid of the invention, once expression of the nucleic acid is no-longer desired in the cell. Similarly, the nucleic acids of the invention, or subsequences or anti-sense sequences thereof, can also be used to block expression of naturally occurring homologous nucleic acids. A variety of sense and anti-sense technologies are known in the art, e.g., as set forth in Lichtenstein and Nellen (1997) Antisense Technology: A Practical Approach IRL Press at Oxford University, Oxford, England, and in Agrawal (1996) Antisense Therepeutics Humana Press, New Jersey, and the references cited therein.

### Kits and Reagents

The present invention is optionally provided to a user as a kit. For example, a kit of the invention contains one or more nucleic acid, polypeptide, antibody, or cell line described herein. Most often, the kit contains a diagnostic nucleic acid or polypeptide, e.g., antibody, probe set, e.g., as a cDNA microarray packaged in a suitable container, or other nucleic acid such as one or more expression vector. The kit typically further comprises, one or more additional reagents, e.g., substrates, labels, primers, for labeling expression products, tubes and/or other accessories, reagents for collecting samples, buffers, hybridization chambers, cover slips, etc. The kit optionally further comprises an instruction set or user manual detailing preferred methods of using the kit components for discovery or application of diagnostic gene sets.

When used according to the instructions, the kit can be used, e.g., for evaluating expression or polymorphisms in a subject sample, i.e., for evaluating breast cancer, for evaluating the type of breast cancer cells, or for evaluating effects of a pharmaceutical agent or hormone intervention on breast cancer progression in a cell or organism.

### Digital Systems

The present invention provides digital systems, e.g., computers, computer readable media and integrated systems comprising character strings corresponding to the sequence information herein for the nucleic acids and isolated or recombinant polypeptides herein, including, e.g., those sequences listed herein and the various silent substitutions and conservative substitutions thereof. Integrated systems can further include, e.g., gene synthesis equipment for making genes corresponding to the character strings.

Various methods known in the art can be used to detect homology or similarity between different character strings, or can be used to perform other desirable functions such as to control output files, provide the basis for making presentations of information including the sequences and the like. Examples include BLAST, discussed supra. Computer systems of the invention can include such programs, e.g., in conjunction with one or more data file or data base comprising a sequence as noted herein.

Thus, different types of homology and similarity of various stringency and length can be detected and recognized in the integrated systems herein. For example, many homology determination methods have been designed for comparative analysis of sequences of biopolymers, for spell-checking in word processing, and for data retrieval from various databases. With an understanding of double-helix pair-wise complement interactions among 4 principal nucleobases in natural polynucleotides, models that simulate annealing of complementary homologous polynucleotide strings can also be used as a foundation of sequence alignment or other operations typically performed on the character strings corresponding to the sequences herein (e.g., word-processing manipulations, construction of figures comprising sequence or subsequence character strings, output tables, etc.).

Thus, standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention by inputting a character string corresponding to one or more polynucleotides and polypeptides of the invention (either nucleic acids or proteins, or both). For example, a system of the invention can include the foregoing software having the appropriate character string information, e.g., used in conjunction with a user interface (e.g., a GUI in a standard operating system such as a Windows, Macintosh or LINUX system) to manipulate strings of characters corresponding to the sequences herein. As noted, specialized alignment programs such as BLAST can also be incorporated into the systems of the invention for alignment of nucleic acids or proteins (or corresponding character strings).

Systems in the present invention typically include a digital computer with data sets entered into the software system comprising any of the sequences herein. The computer can be, e.g., a PC (Intel x86 or Pentium chip compatible DOS™, OS2™ WINDOWS™ WINDOWS NT™, WINDOWS95™, WINDOWS98™ LINUX based machine, a MACINTOSH™, Power PC, or a UNIX based (e.g., SUN™ work station) machine) or other commercially common computer that is known to one of skill. Software for aligning or otherwise manipulating sequences is available, or can easily be constructed by one of skill using a standard programming language such as Visualbasic, PERL, Fortran, Basic, Java, or the like.

Any controller or computer optionally includes a monitor which is often a cathode ray tube (“CRT”) display, a flat panel display (e.g., active matrix liquid crystal display, liquid crystal display), or others. Computer circuitry is often placed in a box which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of sequences to be compared or otherwise manipulated in the relevant computer system.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation of the fluid direction and transport controller to carry out the desired operation.

The software can also include output elements for controlling nucleic acid synthesis (e.g., based upon a sequence or an alignment of a sequences herein), comparisons of samples for differential gene expression or other operations.

In an additional aspect, the present invention provides system kits embodying the methods, composition, systems and apparatus herein. System kits of the invention optionally comprise one or more of the following: (1) an apparatus, system, system component or apparatus component as described herein; (2) instructions for practicing the methods described herein, and/or for operating the apparatus or apparatus components herein and/or for using the compositions herein. In a further aspect, the present invention provides for the use of any apparatus, apparatus component, composition or kit herein, for the practice of any method or assay herein, and/or for the use of any apparatus or kit to practice any assay or method herein.

### Molecular Techniques

In the context of the invention, nucleic acids and/or proteins are manipulated according to well known molecular biology techniques. Detailed protocols for numerous such procedures are described in, e.g., in Ausubel, supra, Sambrook, supra, and Berger, supra.

In addition to the above references, protocols for in vitro amplification techniques, such as the polymerase chain reaction (PCR), the ligase chain reaction (LCR), Qβ-replicase amplification, and other RNA polymerase mediated techniques (e.g., NASBA), useful e.g., for amplifying cDNA probes of the invention, are found in Mullis et al. (1987) U.S. Pat. No. 4,683,202; PCR Protocols A Guide to Methods and Applications (Innis et al. eds) Academic Press Inc. San Diego, Calif. (1990) (“Innis”); Arnheim and Levinson (1990) C&EN 36; The Journal Of NIH Research (1991) 3:81; Kwoh et al. (1989) Proc Natl Acad Sci USA 86, 1173; Guatelli et al. (1990) Proc Natl Acad Sci USA 87:1874; Lomell et al. (1989) J Clin Chem 35:1826; Landegren et al. (1988) Science 241:1077; Van Brunt (1990) Biotechnology 8:291; Wu and Wallace (1989) Gene 4: 560; Barringer et al. (1990) Gene 89:117, and Sooknanan and Malek (1995) Biotechnology 13:563. Additional methods, useful for cloning nucleic acids in the context of the present invention, include Wallace et al. U.S. Pat. No. 5,426,039. Improved methods of amplifying large nucleic acids by PCR are summarized in Cheng et al. (1994) Nature 369:684 and the references therein.

Certain polynucleotides of the invention, e.g., oligonucleotides can be synthesized utilizing various solid-phase strategies involving mononucleotide and/or trinucleotide-based phosphoramidite coupling chemistry. For example, nucleic acid sequences can be synthesized by the sequential addition of activated monomers and/or trimers to an elongating polynucleotide chain. See e.g., Caruthers, M. H. et al. (1992) Meth Enzymol 211:3.

In lieu of synthesizing the desired sequences, essentially any nucleic acid can be custom ordered from any of a variety of commercial sources, such as The Midland Certified Reagent Company (on the World Wide Web at mcrc.com), The Great American Gene Company (on the World Wide Web at genco.com), ExpressGen, Inc. (on the World Wide Web at expressgen.com), Operon Technologies, Inc. (Alameda, Calif.), and many others.

Similarly, commercial sources for nucleic acid and protein microarrays are available, and include, e.g., Affymetrix, Santa Clara, Calif. (on the World Wide Web at affymetrix.com); Agilent, Palo Alto, Calif. (on the World Wide Web at agilent.com); Zyomyx, Hayward, Calif. (on the World Wide Web at zyomyx.com) and Ciphergen Biosciences, Fremont, Calif. (available on the World Wide Web at ciphergen.com).

A variety of techniques can be used to detect differential gene expression and generate the sequence information corresponding to the gene that is differentially expressed. Typically, massively parallel signature sequencing is used; other examples include SAGE data, microarrays and cDNA fragment profiling methods. See, e.g., Brenner et al., (2000), Gene expression analysis by massively parallel signature sequencing (MPSS) on microbead arrays, Nature Biotech., 18:630-634; Tyagi, (2000), Taking a census of mRNA populations with microbeads, Nature Biotech., 18:597-598; Brenner et al., (2000) In vitro cloning of complex mixtures of DNA on microbeads: Physical separation of differentially expressed cDNAs, PNAS USA 97:1665-1670; Okubo et al., (1992), Large scale cDNA sequencing for analysis of quantitative and qualitative aspects of gene expression, Nature Genetics, 2:173-179; Bachem et al., (1996) Visualization of differential gene expression using a novel method of RNA fingerprinting based on AFLP: analysis of gene expression during potato tuber development, Plant J., 9:745-753; Nelson M, et al., (1993) Sequencing two DNA templates in five channels by digital compression, PNAS (US), 90(5): 1647-51; and Shimkets et al., (1999) Gene expression analysis by transcript profiling coupled to database query, Nature Biotechnology, 17:798-803.

Massively parallel signature sequencing (MPSS) is designed for large-scale counting of individual mRNA molecules in a sample. MPSS provides data for all genes in a tissue or cell sample, not just those that have been previously identified and characterized. No prior knowledge of a gene's sequence is required for MPSS; thus, gene expression datasets can be generated from any organism. In addition, MPSS has a high sensitivity level. Anywhere from about 100,000 to about ten million molecules are typically counted in any given sample, so that even genes that are expressed at low levels can be quantified with high accuracy. Typically, an MPSS dataset typically involves greater than, e.g., about 100,000 signature sequences, to about 750,000 signature sequences. Two-flow cells with microbeads initiated with either of two different initiating adaptors can be used for each experiment, e.g., a 2-stepper and 4-stepper as described above. Therefore, datasets containing from about 200,000 to about 1,400,000 signature sequences can be generated for any given sample. The data from multiple MPSS experiments can optionally be combined.

MPSS is a “digital” gene expression tool that counts all mRNA molecules simultaneously. Counting mRNAs with MPSS is based on the ability to uniquely identify every mRNA in a sample. This is done by generating a sequence of 17 or more bases for each mRNA at a specific site upstream from its poly(A) tail (e.g., the last DpnII site in double stranded cDNA). The sequence of 17 or more bases is then used as an mRNA identification “signature.” To measure the level of expression of any given gene in a sample analyzed by MPSS, the total number of signatures for that gene's mRNA are counted.

MPSS signatures for mRNAs in a sample are generated by sequencing double stranded cDNAs fragments cloned on to microbeads using the Lynx Megaclone technology. A clone refers to a single microbead from which 17 or more bases have been sequenced to create a signature sequence tag from an individual cDNA molecule that has been cloned into the Megaclone library. Fragments from 100,000-10,000,000 individual cDNA molecules from a sample are cloned on to 100,000-10,000,000 separate microbeads using, e.g., the procedure described in Brenner et al., supra, PNAS, thereby making a Megaclone library of cloned cDNA fragments.

MPSS and microbead technology is further described in the following patents and references cited within: U.S. Pat. No. 6,306,597 to Macevicz entitled “DNA sequencing by parallel oligonucleotide extensions” issued Oct. 23, 2001; U.S. Pat. No. 6,280,935 to Macevicz entitled “Method of detecting the presence or absence of a plurality of target sequences using oligonucleotide tags” issued Aug. 28, 2001; U.S. Pat. No. 6,265,163 to Albrecht et al., entitled “Solid phase selection of differentially expressed genes” issued Jul. 24, 2001; U.S. Pat. No. 6,235,475 to Brenner et al., entitled “Oligonucleotide tags for sorting and identification” issued May 22, 2001; U.S. Pat. No. 6,228,589 to Brenner entitled “Measurement of gene expression profiles in toxicity determination” issued May 8, 2001; U.S. Pat. No. 6,175,002 to DuBridge et al., entitled “Adaptor-based sequence analysis” issued Jan. 16, 2001; U.S. Pat. No. 6,172,218 to Brenner entitled “Oligonucleotide tags for sorting and identification” issued Jan. 9, 2001; U.S. Pat. No. 6,172,214 to Brenner entitled “Oligonucleotide tags for sorting and identification” issued Jan. 9, 2001; U.S. Pat. No. 6,150,516 to Brenner et al., entitled “Kits for sorting and identifying polynucleotides” issued Nov. 21, 2000; U.S. Pat. No. 6,140,489 to Brenner entitled “Compositions for sorting polynucleotides” issued Oct. 31, 2000; U.S. Pat. No. 6,138,077 to Brenner entitled “Method, apparatus and computer program product for determining a set of non-hybridizing oligonucleotides” issued on Oct. 24, 2000; U.S. Pat. No. 6,013,445 to Albrecht et al., entitled “Massively parallel signature sequencing by ligation of encoded adaptors” issued Jan. 11, 2000; U.S. Pat. No. 5,962,228 to Brenner entitled “DNA extension and analysis with rolling primers” issued Oct. 5, 1999; U.S. Pat. No. 5,888,737 to DuBridge et al., entitled “Adaptor-based sequence analysis” issued Mar. 30, 1999; U.S. Pat. No. 5,780,231 to Brenner entitled “DNA extension and analysis with rolling primers” issued Jul. 14, 1998; U.S. Pat. No. 5,750,341 to Macevicz entitled “DNA sequencing by parallel oligonucleotide extensions” issued May 12, 1998; U.S. Pat. No. 5,747,255 to Brenner entitled “Polynucleotide detection by isothermal amplification using cleavable oligonucleotides” issued May 5, 1998; U.S. Pat. No. 5,969,119 to Macevicz entitled “DNA sequencing by parallel oligonucleotide extensions” issued Oct. 19, 1999; U.S. Pat. No. 5,863,722 to Brenner entitled “Method of sorting polynucleotides” issued Jan. 26, 1999; U.S. Pat. No. 5,846,719 to Brenner et al. entitled “Oligonucleotide tags for sorting and identification” issued Dec. 8, 1998; U.S. Pat. No. 5,763,175 to Brenner entitled “Simultaneous sequencing of tagged polynucleotides” issued Jun. 9, 1998; U.S. Pat. No. 5,695,934 to Brenner entitled “Massively Parallel sequencing of sorted polynucleotides” issued Dec. 9, 1997; U.S. Pat. No. 5,635,400 to Brenner entitled “Minimally cross-hybridizing sets of oligonucleotide tags” issued Jun. 3, 1997; and, U.S. Pat. No. 5,604,097 to Brenner entitled “Methods for sorting polynucleotides using oligonucleotide tags” issued Feb. 19, 1997.

In MPSS, DNA is sequenced through an automated series of adaptor ligations and enzymatic steps. Two, e.g., independent sampling, procedures typically used involve either a 4-stepper or 2-stepper, which differ by using two alternative reading-frame adaptors. For example, in a 4-stepper procedure, the process is initiated by ligating an adaptor molecule to the GATC (DpnII) single-stranded overhangs, and then digesting the samples with BbvI, which is a type IIs restriction enzyme that cuts the DNA at a position 9-13 nucleotides away from the recognition sequence. This produces molecules with a 4 base single stranded overhang immediately adjacent to the DpnII recognition sequence. Another set of adaptors, called encoded adaptors, are hybridized and ligated to the 4 base overhangs on each molecule. The encoded adaptors contain a 4 base single stranded overhang with all possible nucleotide combinations at one end, and a single stranded coded sequence at the other end. One member of the encoded adaptor set will find a partner on the DNA molecules attached to the beads in the flow cell. The exact sequence of each encoded adaptor that hybridizes to the DNA on a microbead is decoded through 16 different sequential hybridization reactions with a set of fluorescent decoder probes. This process yields the first 4 nucleotides at the end of each molecule. To collect additional sequence, the encoded adaptor from the first round is removed by digestion with BbvI, and the process is repeated several times. In the end, a 17 or more -base signature sequence is generated for each bead in the flow-cell. In a 2-stepper, the sequence obtained is in a different reading frame, which is staggered by two bases compared to the 4-stepper.

Specifically, in a 2-stepper protocol, the recognition site for the type IIS restriction enzyme, e.g., BbvI, used to expose the first four nucleotides to identify the signature sequence, is located 11 nucleotides from the GATC site at the end of the adaptor. In the 4-stepper protocol, the recognition site for the type IIS restriction enzyme, e.g., BbvI, used to expose the first four nucleotides to identify the signature sequence, is located 9 nucleotides from the GATC site at the end of the adaptor. The difference between the 2-stepper protocol and the 4-stepper protocol allows the choice of what overhang will be produced after the first restriction enzyme, e.g., BbvI, digestion. The datasets generated with the two different adaptors are different, because a different set of four base-pair overhangs will be generated for each signature sequence depending on whether a 2-stepper or 4-stepper protocol is used. Each exposed four base pair can potentially contain a palindromic structure, e.g., 16 of 256 different possible four base-pair overhangs. There can also be additional biases due to the relative efficiency of individual overhangs in the ligation processes involved during the sequencing cycles. The dataset generated and the biases make the 2-stepper and 4-stepper protocols independent sampling methods.

Ligation-based sequencing is further described in the following patents and references cited within: U.S. Pat. No. 5,714,330 to Brenner et al., entitled “DNA sequencing by stepwise ligation and cleavage” issued Feb. 3, 1998; U.S. Pat. No. 5,599,675 to Brenner entitled “DNA sequencing by stepwise ligation and cleavage” issued Feb. 4, 1997; U.S. Pat. No. 5,831,065 to Brenner entitled “Kits for DNA sequencing by stepwise ligation and cleavage” issued Nov. 3, 1998; U.S. Pat. No. 5,856,093 to Brenner entitled “Method of determining zygosity by ligation and cleavage” issued Jan. 5, 1999; and, U.S. Pat. No. 5,552,278 to Brenner entitled “DNA sequencing by stepwise ligation and cleavage” issued Sep. 3, 1996.

Another technology that can be used is SAGE technology. SAGE is another transcript counting technique that generates a tag sequence for each mRNA. It also generates a digital gene expression profile. SAGE is based on the principles that a short sequence tag derived from a defined position from a mRNA can uniquely identify the transcript and concatenation of the tags allows for high-throughput sequencing. The length of the SAGE tag is about 10 to about 14 nucleotides. The tag sequence is determined using conventional sequencing technologies. See the following publications and references cited within: Velculescu et al., (1995), Serial analysis of gene expression, Science, 270:484-487; and Zhang et al., (1997), Gene expression profiles in normal and cancer cells, Science, 276:1268-1272. To determine expression level of a gene from SAGE technique, the frequency of a sequence tag derived from the corresponding mRNA transcript is measured. As with microarray data described below, adjustments to consider bias and normalization are optionally included in the present invention. See, e.g., Marguiles et al., (2001) Identification and prevention of a GC content bias in SAGE libraries, Nucleic Acid Res., 29(12):E60-0.

Microarrays are also technologies that can be used in the present invention. Typically, a microarray is a solid support that contains a variety of genes. The mRNAs from the sample are then allowed to hybridize to the microarray. Microarrays have the advantage of high throughput analysis of multiple samples. Typically with microarray techniques, some or all of a variety of variables should be considered. These variables include, e.g., that the desired genes are represented on a given array. Second, a microarray exists for the organism of interest. Third, the detection sensitivity is optimized to achieve detection of low expressed genes. Fourth, a sample is compared with a control sample to compensate for several sources of bias and noise in the intensity results. Typically, the experiment is replicated several times to provide a more reliable dataset. Fifth, compensation is made for multiple values for single gene, because multiple values can arise from, e.g., distinct probe sets within different sections within the gene. See Kerr and Churchhill, G. A., (2001), Statistical design and the analysis of gene expression microarray data, Biostatistics, 2:183-201; Wodicka et al., (1997), Genome wide expression monitoring in Saccharomyces cerevisiae, Nature Biotech., 15:1359-1367; Lockhart et al., (1996), Expression monitoring by hybridization to high-density oligonucleotide arrays, Nature Biotech., 14:1675-1680; Aach et al., Systematic management and analysis of yeast gene expression data, Genome Res., 10:431-445 and Wittes and Friedman, (1999) Searching for evidence of altered gene expression: a comment on statistical analysis of microarray data, J. Natl. Cancer Inst., 91:400-401.

More information can be found in the following publications and references cited within: Duggan et al., (1999), Expression profiling using cDNA microarrays, Nature Genetics, 21:10-14; Lipshutz et al., High density synthetic oligonucleotide arrays, Nature Genetics Suppl. 21:20-24; Evertsz et al., (2000), Technology and applications of gene expression microarrays, in Microarray Biochip technology, Schena, M., Ed. BioTechniques Books, Natick, Mass., pp.149-166; Lockhart and Winzeler, (2000), Genomics, gene expression and DNA arrays, Nature, 405:827-836; Zhou et al., (2000), Information processing issues and solutions associated with microarray technology, in Microarray Biochip technology, Schena, M., Ed., BioTechniques Books, Natick, Mass., pp. 167-200; and Hughes et al., (2001), Expression profiling using microarrays fabricated by an ink-jet oligonucleotide synthesizer, Nature Biotech., 19:342-347.

A comparison between two samples can be made in order to determine, e.g., differential expression. A variety of statistical comparison tests can be used, for example, a two-tailed normal approximation test, a chi-squared test, a Fisher exact test, a generalized linear model, Audic and Claverie's Bayesian method and the like. Comparison tests are well-known to one of skill in the art; information on statistical tests can be found in variety of places, such as, textbooks, papers and the World Wide Web. For example, see Fisher and van Belle, (1993) Biostatistics: a Methodology for the Health Science, John Wiley & Sons, New York; Man et al., (2000) POWER SAGE: comparing statistical tests for SAGE experiments, Bioinformatics, 16(11): 953-959; and, Audic and Claverie, (1997) The significance of digital gene expression profiles, Genome Research, 7:986-995. Further details on the use of the two tailed normal approximation test are found in U.S. patent application, concurrently filed on Dec. 10, 2002, LOJAQ docket No. 37-000710US, the contents of which are incorporated by reference.

### EXAMPLES

The following examples are offered to illustrate, but not to limit the claimed invention.

### Example 1

### Identification of Sequence 59-4 That is Differentially Expressed in ER+ Breast Cancer Cells Compared to ER− Breast Cancer Cells

An EST, AW292286 (SEQ ID NO: 1 (“59-4”)), that has never been previously associated with normal mammary gland, breast tumors or breast cancer cell lines, was identified in several breast cancer cell lines (MCF-7, BT-20, MDA-MB-231) using MPSS sequence and Megasort analysis (Lynx Therapeutics, Inc., Hayward, Calif.). The publicly available SAGE databases do not reveal this sequence in normal breast tissue, breast tumors or breast cancer cell lines and this EST is not represented on the Affymetrix U95 GeneChip Set. As a result, 59-4, would not have been discovered by breast cancer researchers using these microarrays. Based on MPSS analysis (28 million clones/million), TaqMan technology and northern analysis, this 59-4 had a low copy number in MCF-7 breast cancer cells. 59-4 encodes a synaptotagmin-like homolog, Slp2a, which has never been reported in the literature as being involved in the etiology or progression of breast cancer or as a potential biomarker distinguishing subsets of ER+ breast cancer cells. Using TaqMan technology, 59-4, was found to be expressed in a subset of ER+ breast cancer cell lines but was absent or significantly downregulated in normal and ER− breast cancer cell lines.

Using Megasort technology, the sequence of 59-4 (SEQ ID NO: 1) was obtained and identified. Specifically, the sequence was obtained and identified by using a bead bed=600,000 beads generated by loading 2 separate libraries, one consisting of cDNA derived from two estrogen receptor negative cell lines (BT-20 and MDA-MB-231) compared to beads loaded with cDNA derived from an estrogen receptor positive cell line (MCF-7). The beads were stripped and subsequently probed with an equal amount of cy5 (ER−) labeled cDNA (5 ug) and FAM (ER+) labeled cDNA (5 ug). 1335 beads were sorted in the cy5 channel (ER−) and 1180 beads were sorted in the FAM channel (ER+). 1000 clones were sequenced from the resulting beads and was analyzed by PCR in each of the 2 different paradigms. 59-4 (SEQ ID NO: 1) was identified 4×from sequencing 1000 clones from the FAM channel.

The 59-4 (SEQ ID NO: 1) sequence was identified with Megasort, genomic mapping, gene specific primers and 5′ RACE:

5′ ATGCTCTTTTCTTATTTAGAAAAGTATTTCTATGTTGCAGATGAACT
GTCTCATTGTGTTGAGCCTGAGCCATCTCAGGTGCCAGGTGGCAGTTCTA
GAGACCGTCAGCAAGGTAAGCCCCCTCCTCTCCCGGCTCTAAAAGCTAAG
ACATCTTCACGTTCTGGTCCATATGCCACTGAGATAAAGAAGTCAACTGA
TGATTCCATATTTAAAGTTCTAGACTGGTTTAACCGAAGTTCTTATTCAG
ATGACAATAAGTCATTCCTCCAACATCCCCGAGGAATAGAGTCCAAAGAA
AAAACAGACTCAAAATCACAGGTTGCTGTTGACTTGGTGACAGATGACAC
TACTTTAAGAGAAAATGGCTCAAAGACCCTATCACCCAGCAAAATTGAAT
TGAAGCCTGTGAGATCTGACTCACCATTCCAAGCAGAGGGAGATATGCTG
GTTTCTGAAAGTTGCCAAGATAATAATGTGAATATCAAATCCAAATTCAT
GAATTTGTCCCAAAAAGGCACCCCAAAGGAAGGCCCAGGTATATTGCAAC
CATTTGAAAGCTATGGCACCCCAAGTCAAGGGAGTAAAAATATGGACTAT
AGCCAAGATTCAAAAAGCCCAGGAAAAGGGAATGGGGCATCTCCTTCAAA
TAGTAACTATTCCTACAGTGTTCTCAAGGAATCTGATGCAGAAAACCAAG
TTCCATGCAACACTAATAATATTGGCAACTTGGGTGAAGAAGAACCCAAG
TTTCATGCTCATGAAGAAAATAGAGGACACTCAGAAGTGAATTTTGACTC
TTCAACAGTTGTCAAAGAACCAGGTTTGAAAGATAACATGAATGCAGAGA
GAAAGAGCAAAGTAGGAAATACCTATATCCTGAAAGCCTCCTTAGAGCCA
GAGAATATTAAGTCAACACCTGGGGTTGCCAACAATGGCTCTCCTTGGAA
GAAGCCTGAGGTCCAATTCCAGCAAGAAGCTGGTGAGGTTCCCAAGAACC
AAGTGCAGAGAGAGAAATACAAAAGAGTGAGTGACAGAATATCCTTTTGG
GAAGGAGAGAAAGCTGGTGCTAAGATAACTCATGAAAAACCCACATCTTC
ATGTAGCCAGGAACAACCTTCTGCTAAAGCATATCAGCCTGTGAAGAAGT
CACAGGGCGTATCATCCATGGACAGTTTATCTACAGACCAGAGTGAATAT
AATCAGGCCATTCCCAAACGAGTGGTCTTAGATGAGGATGATCAAGCATC
CCAGCTCTCCAATTCTTATTCCTCAAATAAATCTAAAGAGACCAAGCCAC
AAATAGCAGGTCCATCCAGATACTATCTTTCAGCTGAGCAATCAGATAAA
GTGTCTCTGTTTCAGAATAAGAAAAATGAGCCTATAAAAAGATCACAAGT
GGCAGACAGTTTGCCTTCTAGAAGAAACATTACTTTACCAGCACTGCAAC
CTCCCTCAAATGTCGGGAGTGAACGACATGCTCCATTGGAGAAAGACAGA
CCTCTAGTTCGTGAATCAAATGCCAACTTTAAAGTTATGTCCCTAAAAGA
AAGAATGGATGAACCCAATGCAGAACAGGTCTATAATCCCTCTCAGTTTG
AGAATTTGAGAAAGTTTTGGGACTTAGAAGCTAATTCAAACAGTAAGGAT
AATGACAAGAATATTACCACCACAAGCCAAAAAAATTCTGCACCTTTTAA
TAGGCAGAAACACAAGGAATTCAGCGACATTAAATTATCAGGTAAAAATA
CCCATGAAGCAGAGGTGCTTCTAAGCCCAAAAAAAGTTATGGCAAGAGAG
GAAATGGAGAAATTAAATTCAAAGGGCATACTCCAGGTGCTACCAGATGA
AATCACATTTCCTTTGAGTCCACTTAGAAAGTATACTTATCAGTTGCCAG
GAAATGAGTCATCAAAGGAAAATGTGGAAAAGAATACGGAAGGGATTGTT
ACTCCAGTGTTTAAGGAAGAAAAGGATTACTCAGAACAAGAGATTCAAGA
ATCCATAATAAAAACCAATGCTTTGTCTAAAGACTGCAAAGACACTTTTA
ATGACAGCTTGCAGAAACTGCTTTCAGAAACCTCAACACCAGCAATTCAA
CCCTCTGGTGGAAAAGTTCATGGAAAACAAGTGCTTGAACCAAGTGTTTC
TGAAAATAGGACATGGCCTCAAAAAACAGATTTTGGTGATACTGAGGAAG
AAGTCAAAGGACCTGAGAAGATCATTAATGAGCATGTTGACAAAACAGTA
GTTCATCCAAAGGTTAAACGGAACTCTTTGACTGCTAGTCTAGACAAACT
CCTGAAGGAAGCAACTGGAACTTCACCCTCTCCCTTGCAAGCCAAGTTGG
CGCCCGTTATCACTGGAACCAACTCTAAGCTGGAAGAGGGGAGATTTTTT
GGAAAAGGGATAGAACAGAGTCACAATACTTCAGCTGATAAGAGAGAAAT
ACTAGCTCCTTTTCCAGTGAGAGATGAAACTTTTGGAAATACAGCTCTCC
TCAAGAAAGCTGAAAGTGGTGAGTGCCAGCTAAGCACACAGAATTTGATT
CAGGTGGCTGCAGAAGATTCTCATCCATTGGATCCAACTTCCCAGCTTTC
CAGAAAGGGTTCTTTTGGGGATGTGGCCAGCCCTCCCCAAGATATGCTTT
TTCCCCAGGGTGCTCATCTTGTTCCCCAGGCTAGGGTACACCCTTCTCAA
ATGGAAATTTCGGAGACTGTAGAGAAAGTCATTCTTCCACCCAGACCTGT
ATTGAATGATGTAAGTGCTGCATTACAGAAGCTGTGTGGAGAAGTATGGT
TAAGTTATCCAGCTGGAAGGGAAGTAGGTCCTGGAGAAGTGAACCCAGAA
TTTCCTGAAGCAGTACAGCCAGTATGTAGCCCCCTAAATCCTCCAGGAGT
GATATCACCATGGGCTACGATGGACACCATAGTTCCAGACAGGAAGGATT
TTTATTCCTCCAATGTAGTTCCTGATAAAACTCATGAAGTTGGATCTTAT
TTAGCTGCCCAAATGTCTCCATCAGACCAGACGCTTAGCTCATTTGCTTC
CATTGTTGCTCAATATGGCAAAGGCCTCCCTCAGGAAGTGGAAGAAATTG
TGAGGGAAACAATTGTTCAACCCAAATCAGAGTTCCTCGAATTCAGTGCT
GGCTTAGAAAAACTACTGAAGGAAGAAACTGAAACCTTCCCCTCAAAATA
TGAAAGTGATACAGGGAATCTTTCTCCATCAAAGTTAATAGGTAGTACAG
AGGAGCCCAGGCGAGCCACTTCTGAATGCCATCCTGAGGAATTAAAAGAA
ACAGTAGAAAAGGCCGAGGCTCCATTAATAACTGAGAGTGCTTTTGATGC
TGGTTTTGAGAAACTTCTTAAAGAAATAACTGAAGCTCCTCCTTATCAGC
CCCAGGTGTCAGTGAGAGAAGAAACTCACGAGAAGGAGTCCTCACAGTCA
GAGCAGACCAGGTTCTTGGGGACAGTGCCCCATTTTTACAGGGCAGCCTC
ACAGACCTCTGAAATGAAGGATAAAAGTAATGGTTTGGAATCTCAAGTCA
ACCAATGTGATAAAATGTTGGGAGGAGACGCACTTGTGACTGATTTATTG
GTAGATTTTTGTGGTTCCAGAAGTGGAGTTGAGATCCCTAGAACCCCACA
ACTTTATGTGGCTCATGAAATAGGGACCATTAAAACTGTAACCCCCCCAG
AGGACAGGGACAGTGAAAGTGGGGTTGCAGGGGGACAAGGGACTCTTCAG
GAACCTGGCTTTGGAGAGGCTTCTGAAGCAATTAGTGTGTCCAGAAATAG
GCAACCCATTCCTCTCCTGATGAACAAAGAAAACTCTACAAAAACAAGTA
AAGTTGAATTGACTCTAGCATCGCCATATATGAAACAAGAGAAAGAGGAA
GAAAAAGAAGGTTTCTCTGAGTCTGATTTTTCAGATGGAAACACCAGTTC
TAATGCAGAGAGCTGGAGAAATCCTTCCAGTGAGCATTTAAAATTTTTTA
ACTCACAGTGAGTCTGCTTTATGGAATGCTGATGTAAAGGATGATTTTTT
TTTAATTGGGATTAATTTGATATTCTTAAATGATGTTAAATTCTTCTTAC
CACTTCTGTGTTCCTGAGACTTTCTTAATTAAAAAAATTATTTATATTTT
TTAATCAAATGCATTACATAATTCTTAAATGATGTTAAATTCTTCTTACC
ACTTCTGTGTTCCTGAGACTTTCTTAATTAAAAAAATTATTTATATTTTT
TAATCAAATGCATTACGTAAAACAGCTTTCCTATAAGGCTGTTTCAGAGT
CTGAGTTGACTTCTCTTTAATCTACCTATAGAACTTTTAGGTTTCAAAAA
ATACTTTTTAAATGACTTTTTGGGTTTGGAAAGTACCTTTAATACATTTA
AGCTAGTTTTCCTCCTGGAAATATTTAGAATTTCTTCCTTAATTGGCAAC
CTTTATAGAAGTCTGGTAAGATTTGTCGCAAAGATGTGCCACAGATGGAC
ACAAATTTCCCATTCGGGAGCAATATCTTACCACAGTGGTGGCTAAATGC
TAGGGACAAAATACAAGGCCGGAACTTTCCTTCCCTCAGATACCTTGTGC
TGTGGTGTTTTGTTGCCACTTTCTCCCTCTCATTTTCAATTATATGCACA
ATCTTCCCTTTCTAGAGTATGACTTTGGCCAGATGACTCACCTGATGCCA
CCTAAGGGCATTGCCTGGCCAGGTACATTTCTCTGGCTCCAGCCTTGGCT
AAGTTGATGACCTGAGTCGATCTCCACATTCATCTACATGAACGTGGGGG
CGTTGGTTTTGGCGGCCAGGCTGTAAAATGTAGGGCTTGTCTCAGTTTGC
TATTTAATCAACATGTGGACATTTTAGCAGAGAAACCCCAGAGCAAATAG
GAATGAGAAGCTACCTGATTAAAATGATGAAATGATAGAGAATGTTTTTT
GGCTGGGACATTTTAACCAAAGTTGCACAACTGATGCTGATTGCCTTCCT
TGTAGTTTAGTAGAATTTGTCATTTGTTTAGCTCCTTTTCGTTCCAGTGA
AAATAGATAAGCTTTACT-3′

The sequence obtained by MPSS is underlined and in bold and the sequence obtained by Megasort follows the MPSS sequence.

MPSS also identified the 59-4 sequence as being differentially expressed in ER+ breast cancer cells lines verses ER− breast cancer cell lines. Using MPSS, beads from different breast cancer cells lines were generated by loading 4 separate cDNA libraries (MCF-7, BT-20, MDA-MB-231 and a mixed cDNA population containing BT-20 and MDA-MB-231) onto the beads. The following number of beads were sequenced using 2-stepper and 4-stepper MPSS methodologies: MCF-7=1.5 million, BT-20=1.2 million, MDA-MB-231=731,282, and ER− (BT-20 & MDA-MB-231)=276,807. The signature representing 59-4: GATCTCCACATTCATCT, was identified in MCF-7 cDNA but not found in any other cDNA sample. From these experiments, the normalized value/million of 59-4 expression in MCF-7 was 28 beads/million. Normal mammary gland was also sequenced from cDNA obtained commercially from Clontech and 1.4 million clones were sequenced. This was considered the “normal” breast sample for comparisons between non-malignant and malignant breast cancer cell lines.

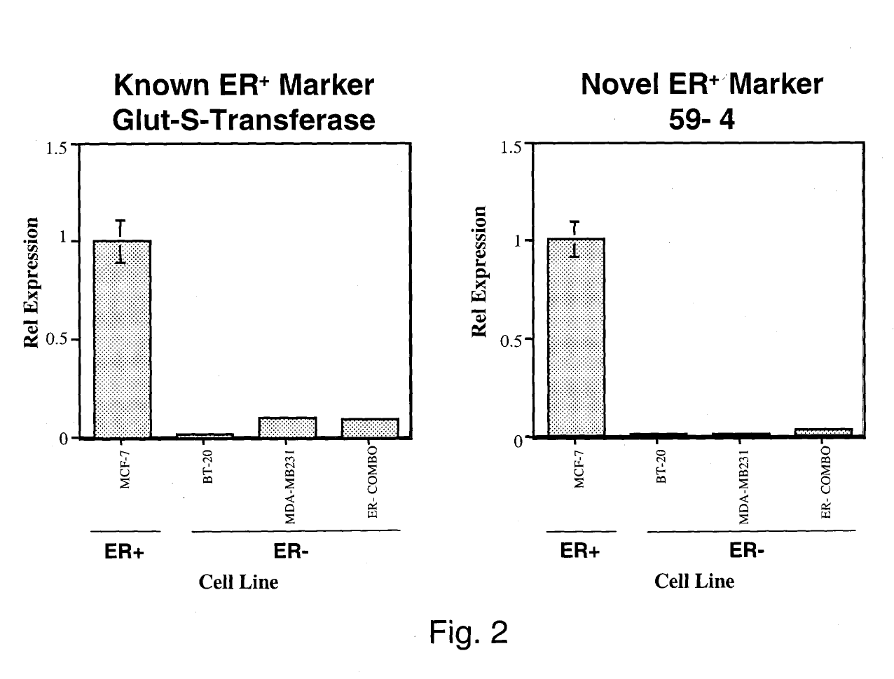

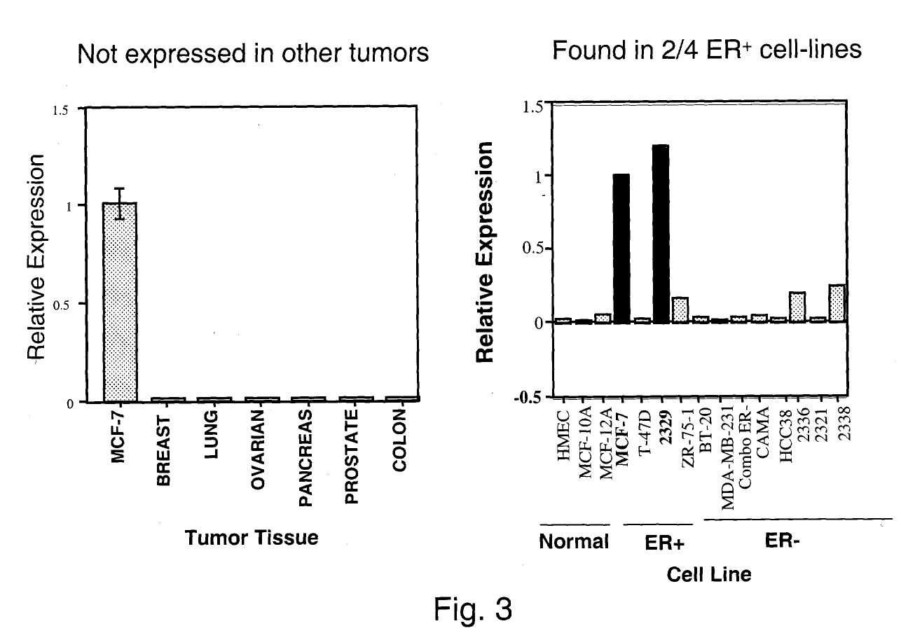

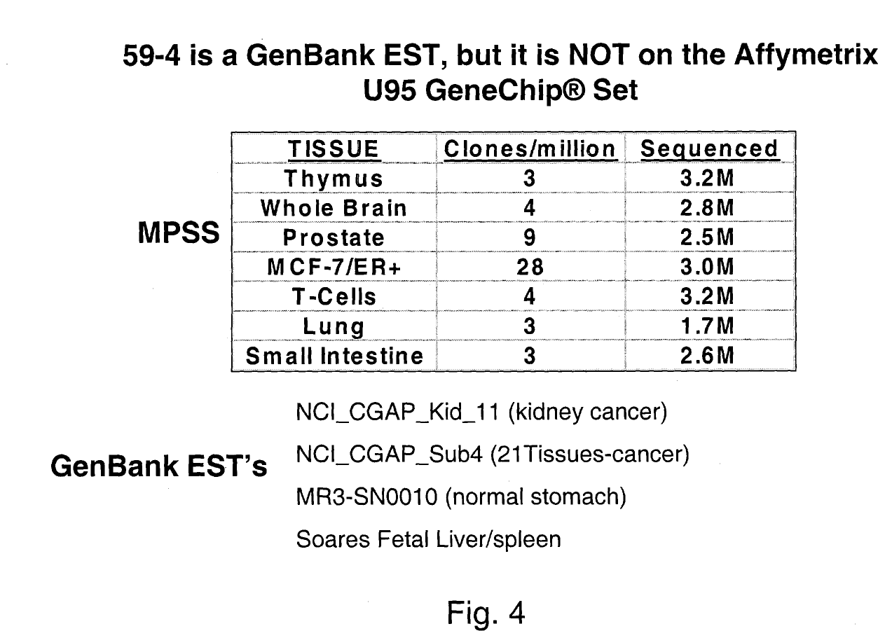

The expression of 59-4 was assayed in 3 normal (HMEC, MCF-10A, MCF-12A), 4 ER+ (MCF-7, T-47D, 2329, ZR-75-1, BT-474, and SKBR-3) and 7 ER− (BT-20, MDA-MB-231, CAMA, HCC38, 2336, 2321, and 2338) cell lines. Expression analysis using quantitative RT-PCR indicated that 59-4 was expressed in 2 out of the 6 estrogen-receptor positive (ER+) breast cancer cells lines, but was absent or significantly down-regulated in 4 normal mammary epithelial cell-lines and 7 estrogen receptor negative (ER−) breast cancer cell lines. Using a set of known genes reported in the literature to be differentially expressed between ER+ and ER− cell lines and primary tumors, 93% of these sequences were identified. Furthermore, 93% of these known genes had the appropriate differential regulation as published in the literature. For example, FIG. 2 illustrates 59-4 expression along with Glutathione-S-Transferase, a known ER+ marker, in ER+ and ER− cell lines. cDNA was prepared from primary tumor tissue from several different tissues, breast, lung, ovarian, pancreas, prostate, and colon and tested for 59-4 expression. No detection of expression of 59-4 was found in these tumors. See FIG. 3 which illustrates 59-4 expression in tumors and cell lines. As a result, 59-4 is not associated with the etiology or progression of this subset of human tumor tissue, but can be specific to a particular stage of breast cancer. The tissue specific expression of 59-4 was examined using a Lynx human tissue database. It was found that 59-4 is also expressed at very low levels in the following normal tissues: thymus, whole brain, prostate, T-cells, lung and small intestine. See FIG. 4 which illustrates tissue specific expression of 59-4. In the human EST database, 59-4 has never been identified in any of these tissues, further demonstrating the sensitivity of the MPSS technology.

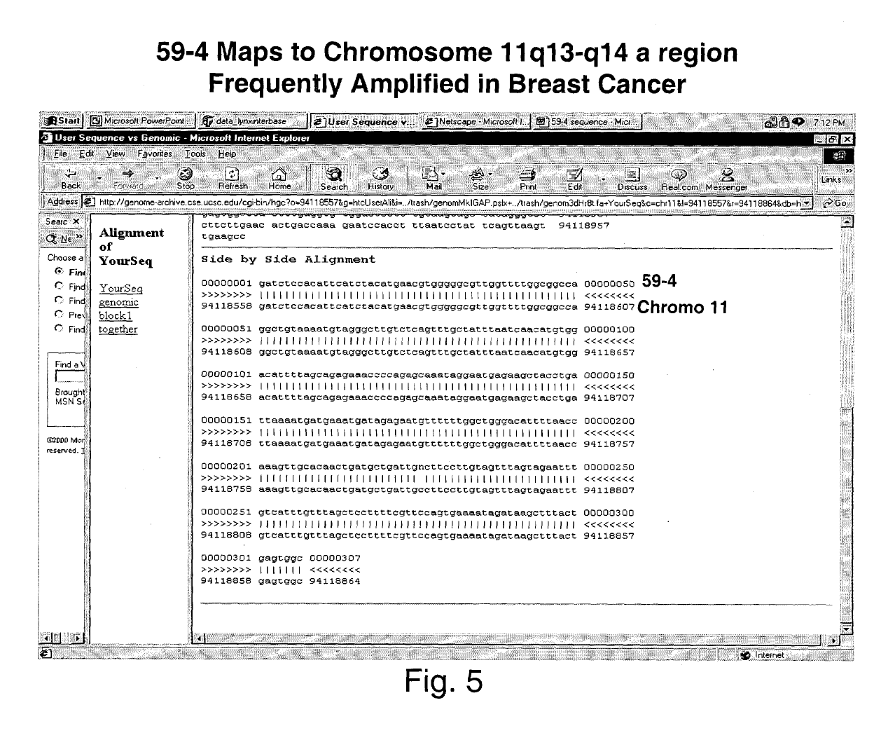

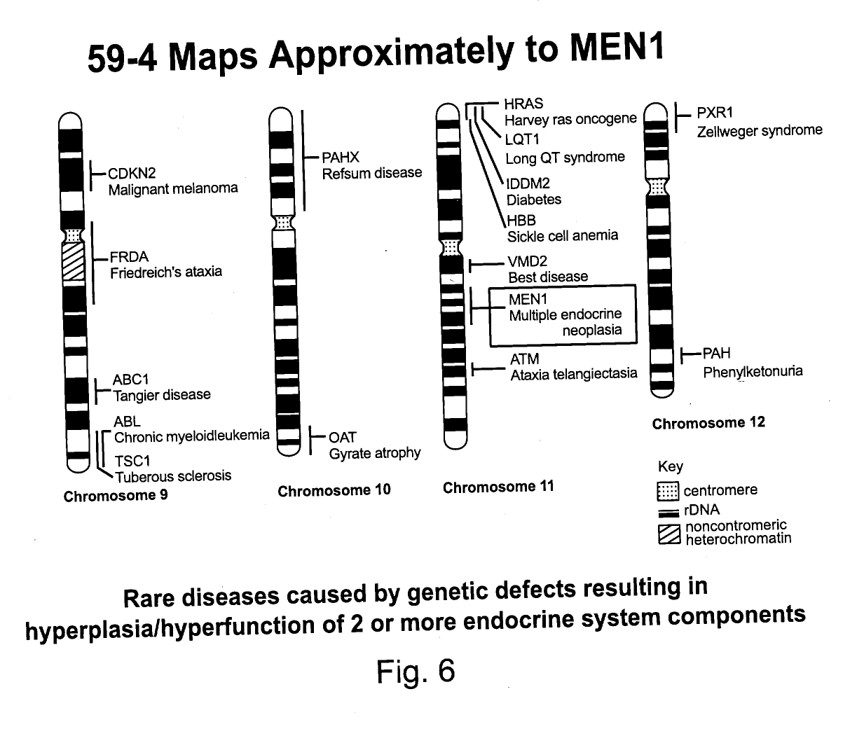

Chromosome 11 contains a region that is commonly amplified in human breast carcinoma. Specifically, the amplification units described on 11q13→q14 contain an area that is amplified in a subset of estrogen receptor positive carcinomas that are prone to metastasis. See, e.g., Bekri et al., (1997) Detailed map of a region commonly amplified at 11q12→q14 in human breast carcinoma, Cytogenet. Cell Genet. 79:125-131. Other chromosomes, such as 17 and 20, are also implicated in containing regions that are amplified in breast cancer tumors. Blast analysis of the 59-4 sequence against the UC Santa Cruz human genome database revealed a match at chromosome 11q13-q14. See FIG. 5 which illustrates the mapping of 59-4 to Chromosome 11q13-q14 and FIG. 6 which illustrates 59-4 mapping to approximately MEN1. A predicted ORF from an mRNA, FLJ20163, was found just 5′ to the 59-4 sequence. Using 5′ RACE with MCF-7 cDNA, the 5′ sequence was extended to within ˜1 kb of the 5′ end of the predicted FLJ20163 ORF. Blast analysis against protein databases revealed homology with a mouse synaptotagmin-like protein slp2a.

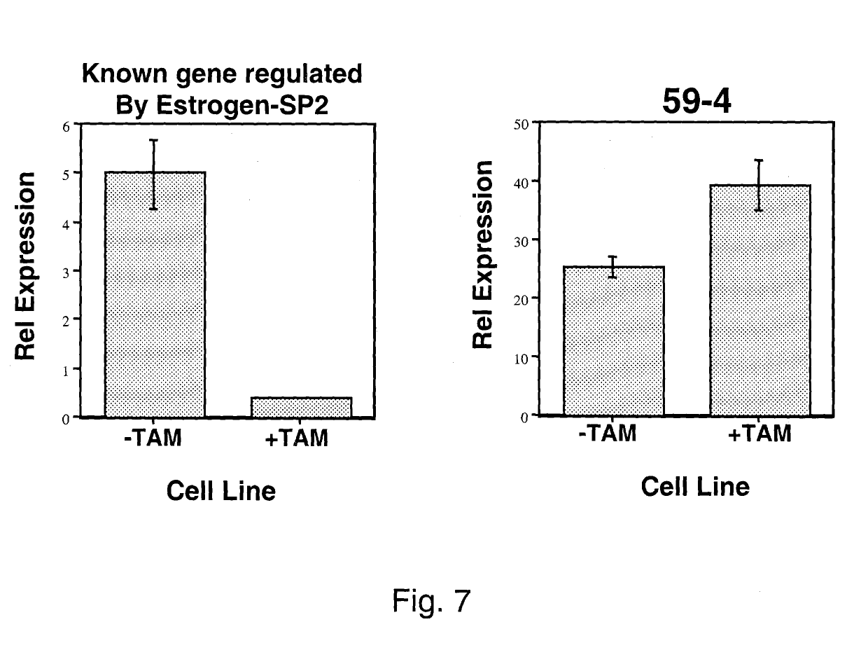

The effect of anti-estrogen Tamoxifen on the expression of 59-4 in MCF-7 cells was examined. ER+ mediates the therapeutic effects of tamoxifen. It has been shown previously that one of the mechanisms of action of Tamoxifen is to act as a competitive inhibitor of estrogen binding to the estrogen receptor. Treatment of MCF-7 cells with 10⁻⁶ M Tamoxifen inhibited the expression of a known estrogen-regulated gene, SP2, however, 59-4 was upregulated ˜1.6 fold by TaqMan analysis. FIG. 7 illustrates that Tamoxifen (TAM) increases the expression of 59-4 in a cell line. The polypeptide encoded by 59-4 is found as SEQ ID NO: 492.

### Example 2

### Identification of Sequences That are Differentially Expressed in ER+ Breast Cancer Cells Compared to ER− Breast Cancer Cells

Hierarchical clustering can be used to group genes based on their similarity of expression. See, e.g., Perou, supra; Eisen et al., (1998), Cluster analysis and display of genome-wide expression patterns, Proc. Natl. Acad. Sci. USA, 95: 14863-14868; and Bertucci et al., (2000), Gene expression profiling of primary breast carcinomas using arrays of candidate genes, Human Molecular Genetics, 9(20): 2981-2991. Patterns of the gene expression can provide molecular portraits of the cells, tissue and/or tumor.

Using a heirararchical clustering scheme, described above, 285 signatures sequences, SEQ ID NO: 2-SEQ ID NO: 286, were identified from a Lynx human tissue database as having a similar expression pattern to 59-4 (SEQ ID NO.: 1). These sequences are: 1) expressed in an estrogen-receptor positive (ER+) breast cancer cell line, MCF-7; 2) absent from several ER− cells lines, e.g., BT-20 and MDA-MB-231, and normal mammary gland tissue (MG); and 3) expressed at very low levels in 21 other human tissues. See SEQ ID Nos: 2-286 and Appendix A for listing of the 285 sequences. Information on the sequences can be found in Appendix B and Appendix C.

### Example 3

### Identification of Sequences That are Breast Cancer Stage Specific Markers

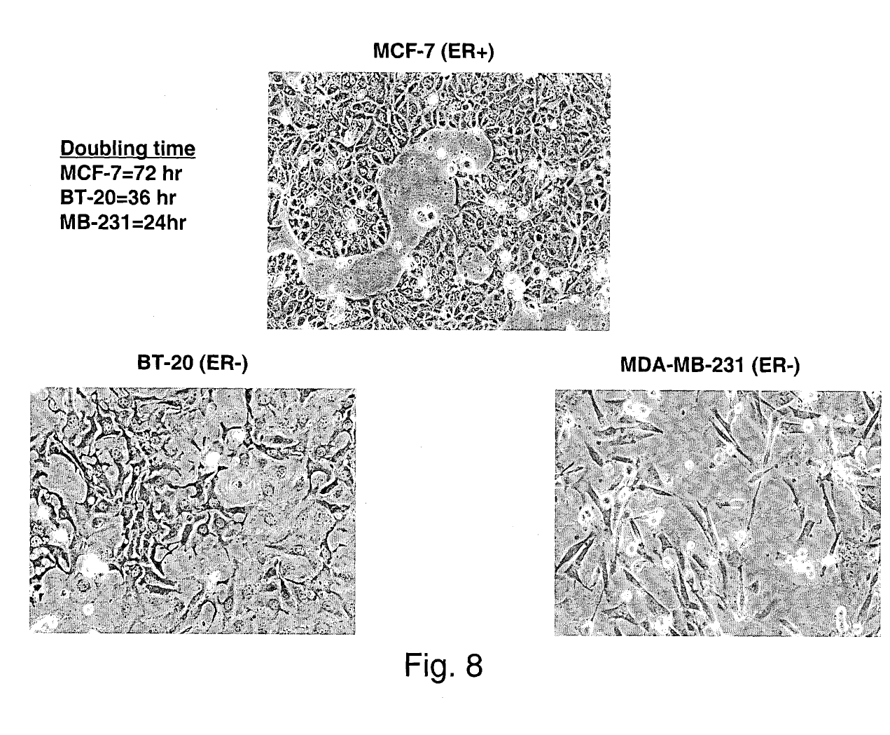

The following pairwise comparisons: MCF-7 vs MG, BT-20 vs MG, MDA-MB-231 vs MG, MCF-7 vs BT-20, MCF-7 vs MDA-MB-231, MDA-MB-231 vs BT-20 were used to find sequences that were differentially expressed in breast cancer cells compared to normal mammary epithelia and/or differentially expressed in different stages of cancer using a statistical analysis of the data. MG represents cells of normal mammary gland tissue. MCF-7 is an ER+ cancer cell lines. BT-20 and MDA-MD-231 are ER− breast cancer cell lines. See FIG. 8 that shows contrast photographs of three different human breast cancer cell lines, MCF-7, BT-20 and MDA-MB-231. Specifically, sequences that were statistically significant, p<0.01 in pairwise comparisons were identified. From this analysis, 12,967 unique signatures were identified within this paradigm in at least 1 of 6 pairwise comparisons. 205 signatures (e.g., SEQ ID NO: 287-491) are provided that were significantly different in all of the pairwise combinations. These signatures along with the ones in Examples 1 and 2 can be used to as biomarkers distinguishing the various phenotypes of breast cancer. The sequences are listed as SEQ ID Nos: 287-491 and also found in Appendix D.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Sequence Listing

| SEQ ID NO: # | CLONE ID | SEQUENCE |
|---|---|---|
| SEQ ID: 1 | 59-4 | 5′ ATGCTCTTTTCTTATTTAGAAAAGTATTTCTATGTTGCAGATG |
| | | AACTGTCTCATTGTGTTGAGCCTGAGCCATCTCAGGTGCCAGGT |
| | | GGCAGTTCTAGAGACCGTCAGCAAGGTAAGCCCCCTCCTCTCCC |
| | | GGCTCTAAAAGCTAAGACATCTTCACGTTCTGGTCCATATGCCA |
| | | CTGAGATAAAGAAGTCAACTGATGATTCCATATTTAAAGTTCTA |
| | | GACTGGTTTAACCGAAGTTCTTATTCAGATGACAATAAGTCATT |
| | | CCTCCAACATCCCCGAGGAATAGAGTCCAAAGAAAAAACAGAC |
| | | TCAAAATCACAGGTTGCTGTTGACTTGGTGACAGATGACACTAC |
| | | TTTAAGAGAAAATGGCTCAAAGACCCTATCACCCAGCAAAATT |
| | | GAATTGAAGCCTGTGAGATCTGACTCACCATTCCAAGCAGAGG |
| | | GAGATATGCTGGTTTCTGAAAGTTGCCAAGATAATAATGTGAAT |
| | | ATCAAATCCAAATTCATGAATTTGTCCCAAAAAGGCACCCCAA |
| | | AGGAAGGCCCAGGTATATTGCAACCATTTGAAAGCTATGGCAC |
| | | CCCAAGTCAAGGGAGTAAAAATATGGACTATAGCCAAGATTCA |
| | | AAAAGCCCAGGAAAAGGGAATGGGGCATCTCCTTCAAATAGTA |
| | | ACTATTCCTACAGTGTTCTCAAGGAATCTGATGCAGAAAACCAA |
| | | GTTCCATGCAACACTAATAATATTGGCAACTTGGGTGAAGAAG |
| | | AACCCAAGTTTCATGCTCATGAAGAAAATAGAGGACACTCAGA |
| | | AGTGAATTTTGACTCTTCAACAGTTGTCAAAGAACCAGGTTTGA |
| | | AAGATAACATGAATGCAGAGAGAAAGAGCAAAGTAGGAAATA |
| | | CCTATATCCTGAAAGCCTCCTTAGAGCCAGAGAATATTAAGTCA |
| | | ACACCTGGGGTTGCCAACAATGGCTCTCCTTGGAAGAAGCCTG |
| | | AGGTCCAATTCCAGCAAGAAGCTGGTGAGGTTCCCAAGAACCA |
| | | AGTGCAGAGAGAGAAATACAAAAGAGTGAGTGACAGAATATC |
| | | CTTTTGGGAAGGAGAGAAAGCTGGTGCTAAGATAACTCATGAA |
| | | AAACCCACATCTTCATGTAGCCAGGAACAACCTTCTGCTAAAGC |
| | | ATATCAGCCTGTGAAGAAGTCACAGGGCGTATCATCCATGGAC |
| | | AGTTTATCTACAGACCAGAGTGAATATAATCAGGCCATTCCCAA |
| | | ACGAGTGGTCTTAGATGAGGATGATCAAGCATCCCAGCTCTCCA |
| | | ATTCTTATTCCTCAAATAAATCTAAAGAGACCAAGCCACAAATA |
| | | GCAGGTCCATCCAGATACTATCTTTCAGCTGAGCAATCAGATAA |
| | | AGTGTCTCTGTTTCAGAATAAGAAAAATGAGCCTATAAAAAGA |
| | | TCACAAGTGGCAGACAGTTTGCCTTCTAGAAGAAACATTACTTT |
| | | ACCAGCACTGCAACCTCCCTCAAATGTCGGGAGTGAACGACAT |
| | | GCTCCATTGGAGAAAGACAGACCTCTAGTTCGTGAATCAAATG |
| | | CCAACTTTAAAGTTATGTCCCTAAAAGAAAGAATGGATGAACC |
| | | CAATGCAGAACAGGTCTATAATCCCTCTCAGTTTGAGAATTTGA |
| | | GAAAGTTTTGGGACTTAGAAGCTAATTCAAACAGTAAGGATAA |
| | | TGACAAGAATATTACCACCACAAGCCAAAAAAATTCTGCACCT |
| | | TTTAATAGGCAGAAACACAAGGAATTCAGCGACATTAAATTAT |
| | | CAGGTAAAAATACCCATGAAGCAGAGGTGCTTCTAAGCCCAAA |
| | | AAAAGTTATGGCAAGAGAGGAAATGGAGAAATTAAATTCAAAG |
| | | GGCATACTCCAGGTGCTACCAGATGAAATCACATTTCCTTTGAG |
| | | TCCACTTAGAAAGTATACTTATCAGTTGCCAGGAAATGAGTCAT |
| | | CAAAGGAAAATGTGGAAAAGAATACGGAAGGGATTGTTACTCC |
| | | AGTGTTTAAGGAAGAAAAGGATTACTCAGAACAAGAGATTCAA |
| | | GAATCCATAATAAAAACCAATGCTTTGTCTAAAGACTGCAAAG |
| | | ACACTTTTAATGACAGCTTGCAGAAACTGCTTTCAGAAACCTCA |
| | | ACACCAGCAATTCAACCCTCTGGTGGAAAAGTTCATGGAAAAC |
| | | AAGTGCTTGAACCAAGTGTTTCTGAAAATAGGACATGGCCTCA |
| | | AAAAACAGATTTTGGTGATACTGAGGAAGAAGTCAAAGGACCT |
| | | GAGAAGATCATTAATGAGCATGTTGACAAAACAGTAGTTCATC |
| | | CAAAGGTTAAACGGAACTCTTTGACTGCTAGTCTAGACAAACTC |
| | | CTGAAGGAAGCAACTGGAACTTCACCCTCTCCCTTGCAAGCCAA |
| | | GTTGGCGCCCGTTATCACTGGAACCAACTCTAAGCTGGAAGAG |
| | | GGGAGATTTTTTGGAAAAGGGATAGAACAGAGTCACAATACTT |
| | | CAGCTGATAAGAGAGAAATACTAGCTCCTTTTCCAGTGAGAGA |
| | | TGAAACTTTTGGAAATACAGCTCTCCTCAAGAAAGCTGAAAGT |
| | | GGTGAGTGCCAGCTAAGCACACAGAATTTGATTCAGGTGGCTG |
| | | CAGAAGATTCTCATCCATTGGATCCAACTTCCCAGCTTTCCAGA |
| | | AAGGGTTCTTTTGGGGATGTGGCCAGCCCTCCCCAAGATATGCT |
| | | TTTTCCCCAGGGTGCTCATCTTGTTCCCCAGGCTAGGGTACACC |
| | | CTTCTCAAATGGAAATTTCGGAGACTGTAGAGAAAGTCATTCTT |
| | | CCACCCAGACCTGTATTGAATGATGTAAGTGCTGCATTACAGAA |
| | | GCTGTGTGGAGAAGTATGGTTAAGTTATCCAGCTGGAAGGGAA |
| | | GTAGGTCCTGGAGAAGTGAACCCAGAATTTCCTGAAGCAGTAC |
| | | AGCCAGTATGTAGCCCCCTAAATCCTCCAGGAGTGATATCACCA |
| | | TGGGCTACGATGGACACCATAGTTCCAGACAGGAAGGATTTTT |
| | | ATTCCTCCAATGTAGTTCCTGATAAAACTCATGAAGTTGGATCT |
| | | TATTTAGCTGCCCAAATGTCTCCATCAGACCAGACGCTTAGCTC |
| | | ATTTGCTTCCATTGTTGCTCAATATGGCAAAGGCCTCCCTCAGG |
| | | AAGTGGAAGAAATTGTGAGGGAAACAATTGTTCAACCCAAATC |
| | | AGAGTTCCTCGAATTCAGTGCTGGCTTAGAAAAACTACTGAAG |
| | | GAAGAAACTGAAACCTTCCCCTCAAAATATGAAAGTGATACAG |
| | | GGAATCTTTCTCCATCAAAGTTAATAGGTAGTACAGAGGAGCCC |
| | | AGGCGAGCCACTTCTGAATGCCATCCTGAGGAATTAAAAGAAA |
| | | CAGTAGAAAAGGCCGAGGCTCCATTAATAACTGAGAGTGCTTT |
| | | TGATGCTGGTTTTGAGAAACTTCTTAAAGAAATAACTGAAGCTC |
| | | CTCCTTATCAGCCCCAGGTGTCAGTGAGAGAAGAAACTCACGA |
| | | GAAGGAGTCCTCACAGTCAGAGCAGACCAGGTTCTTGGGGACA |
| | | GTGCCCCATTTTTACAGGGCAGCCTCACAGACCTCTGAAATGAA |
| | | GGATAAAAGTAATGGTTTGGAATCTCAAGTCAACCAATGTGAT |
| | | AAAATGTTGGGAGGAGACGCACTTGTGACTGATTTATTGGTAG |
| | | ATTTTTGTGGTTCCAGAAGTGGAGTTGAGATCCCTAGAACCCCA |
| | | CAACTTTATGTGGCTCATGAAATAGGGACCATTAAAACTGTAAC |
| | | CCCCCCAGAGGACAGGGACAGTGAAAGTGGGGTTGCAGGGGG |
| | | ACAAGGGACTCTTCAGGAACCTGGCTTTGGAGAGGCTTCTGAA |
| | | GCAATTAGTGTGTCCAGAAATAGGCAACCCATTCCTCTCCTGAT |
| | | GAACAAAGAAAACTCTACAAAAACAAGTAAAGTTGAATTGACT |
| | | CTAGCATCGCCATATATGAAACAAGAGAAAGAGGAAGAAAAA |
| | | GAAGGTTTCTCTGAGTCTGATTTTTCAGATGGAAACACCAGTTC |
| | | TAATGCAGAGAGCTGGAGAAATCCTTCCAGTGAGCATTTAAAA |
| | | TTTTTTAACTCACAGTGAGTCTGCTTTATGGAATGCTGATGTAA |
| | | AGGATGATTTTTTTTTAATTGGGATTAATTTGATATTCTTAAATG |
| | | ATGTTAAATTCTTCTTACCACTTCTGTGTTCCTGAGACTTTCTTA |
| | | ATTAAAAAAATTATTTATATTTTTTAATCAAATGCATTACATAA |
| | | TTCTTAAATGATGTTAAATTCTTCTTACCACTTCTGTGTTCCTGA |
| | | GACTTTCTTAATTAAAAAAATTATTTATATTTTTTAATCAAATGC |
| | | ATTACGTAAAACAGCTTTCCTATAAGGCTGTTTCAGAGTCTGAG |
| | | TTGACTTCTCTTTAATCTACCTATAGAACTTTTAGGTTTCAAAAA |
| | | ATACTTTTTAAATGACTTTTTGGGTTTGGAAAGTACCTTTAATAC |
| | | ATTTAAGCTAGTTTTCCTCCTGGAAATATTTAGAATTTCTTCCTT |
| | | AATTGGCAACCTTTATAGAAGTCTGGTAAGATTTGTCGCAAAGA |
| | | TGTGCCACAGATGGACACAAATTTCCCATTCGGGAGCAATATCT |
| | | TACCACAGTGGTGGCTAAATGCTAGGGACAAAATACAAGGCCG |
| | | GAACTTTCCTTCCCTCAGATACCTTGTGCTGTGGTGTTTTGTTGC |
| | | CACTTTCTCCCTCTCATTTTCAATTATATGCACAATCTTCCCTTT |
| | | CTAGAGTATGACTTTGGCCAGATGACTCACCTGATGCCACCTAA |
| | | GGGCATTGCCTGGCCAGGTACATTTCTCTGGCTCCAGCCTTGGC |
| | | TAAGTTGATGACCTGAGTCGATCTCCACATTCATCTACATGAAC |
| | | GTGGGGGCGTTGGTTTTGGCGGCCAGGCTGTAAAATGTAGGGC |
| | | TTGTCCTCAGTTTGCTATTTAATCAACATGTGGACATTTTAGCAG |
| | | AGAAACCCCAGAGCAAATAGGAATGAGAAGCTACCTGATTAAA |
| | | ATGATGAAATGATAGAGAATGTTTTTTGGCTGGGACATTTTAAC |
| | | CAAAGTTGCACAACTGATGCTGATTGCCTTCCTTGTAGTTTAGT |
| | | AGAATTTGTCATTTGTTTAGCTCCTTTTCGTTCCAGTGAAAATAG |
| | | ATAAGCTTTACT-3′ |
| SEQ ID: 2 | 285-1 | GATCAAAACATAGAAT |
| SEQ ID: 3 | 285-2 | GATCAAAAGTTCTAAT |
| SEQ ID: 4 | 285-3 | GATCAAAACATTCTGCA |
| SEQ ID: 5 | 285-4 | GATCACTCTGTGCACAC |
| SEQ ID: 6 | 285-5 | GATCAAAACTGTCTTCC |
| SEQ ID: 7 | 285-6 | GATCAAACCGAGGCCCA |
| SEQ ID: 8 | 285-7 | GATCAAAGGAAGAAGGC |
| SEQ ID: 9 | 285-8 | GATCAGAAGTATTGCTT |
| SEQ ID: 10 | 285-9 | GATCAAAGGTTGTCAAT |
| SEQ ID: 11 | 285-10 | GATCAAAGTACTCTAGG |
| SEQ ID: 12 | 285-11 | GATCAACTATATGTGAA |
| SEQ ID: 13 | 285-12 | GATCCTAGGATTTTTTT |
| SEQ ID: 14 | 285-13 | GATCATGCTACTATCCT |
| SEQ ID: 15 | 285-14 | GATCAAGTTTAAACAAA |
| SEQ ID: 16 | 285-15 | GATCAAGAATCTTATGT |
| SEQ ID: 17 | 285-16 | GATCTCCACGTTCCTGT |
| SEQ ID: 18 | 285-17 | GATCAAGATATTCTGGT |
| SEQ ID: 19 | 285-18 | GATCCCTCCTGTGATAC |
| SEQ ID: 20 | 285-19 | GATCAGTGAGTGAAGCA |
| SEQ ID: 21 | 285-20 | GATCAGCCTGGGTGGGA |
| SEQ ID: 22 | 285-21 | GATCCCCGATGGTGAGC |
| SEQ ID: 23 | 285-22 | GATCACAGAGCACTGCT |
| SEQ ID: 24 | 285-23 | GATCACCTGCATGGGTC |
| SEQ ID: 25 | 285-24 | GATCACAGCAGGAGCCA |
| SEQ ID: 26 | 285-25 | GATCACTGCGAATTACG |
| SEQ ID: 27 | 285-26 | GATCACCTAACATAACA |
| SEQ ID: 28 | 285-27 | GATCACCTTTTGGAGCA |
| SEQ ID: 29 | 285-28 | GATCAGATGAATCTCAA |
| SEQ ID: 30 | 285-29 | GATCTCTTCGCCCGCAG |
| SEQ ID: 31 | 285-30 | GATCTCTTCGCCCGCAG |
| SEQ ID: 32 | 285-31 | GATCACGCCCCTCTCAC |
| SEQ ID: 33 | 285-32 | GATCATGGAGGACTGGT |
| SEQ ID: 34 | 285-33 | GATCAGCGCCTGGATAT |
| SEQ ID: 35 | 285-34 | GATCACGCCACTGTTCT |
| SEQ ID: 36 | 285-35 | GATCACTGGAGCCCAAA |
| SEQ ID: 37 | 285-36 | GATCAGAAGAGCTGGTG |
| SEQ ID: 38 | 285-37 | GATCAGCACGCCATTCG |
| SEQ ID: 39 | 285-38 | GATCAGCGGTATTGAAG |
| SEQ ID: 40 | 285-39 | GATCAGGAGGCATGAAT |
| SEQ ID: 41 | 285-40 | GATCATGTTATCTATTT |
| SEQ ID: 42 | 285-41 | GATCCAAAGCCCATGTG |
| SEQ ID: 43 | 285-42 | GATCCACACATCTACCT |
| SEQ ID: 44 | 285-43 | GATCCACTCCTGGTGGG |
| SEQ ID: 45 | 285-44 | GATCCAGGCACCCAGCC |
| SEQ ID: 46 | 285-45 | GATCATGTTATCTGGTT |
| SEQ ID: 47 | 285-46 | GATCATTGTTAGCATTT |
| SEQ ID: 48 | 285-47 | GATCCAGTGTATTTTCT |
| SEQ ID: 49 | 285-48 | GATCATTTATGTGTTTT |
| SEQ ID: 50 | 285-49 | GATCCCAAGGTGGCAGG |
| SEQ ID: 51 | 285-50 | GATCCCATTACATTGTA |
| SEQ ID: 52 | 285-51 | GATCACTGCCTCTCCCC |
| SEQ ID: 53 | 285-52 | GATCAGGCGGTGCCAAT |
| SEQ ID: 54 | 285-53 | GATCCATGAGGTTCACC |
| SEQ ID: 55 | 285-54 | GATCATGCGGGTAACCC |
| SEQ ID: 56 | 285-55 | GATCAGACCAGCTGGGG |
| SEQ ID: 57 | 285-56 | GATCTTCAGGGCAACTC |
| SEQ ID: 58 | 285-57 | GATCATCAAGTCCACAA |
| SEQ ID: 59 | 285-58 | GATCAGAGTTCTGCCTG |
| SEQ ID: 60 | 285-59 | GATCATTTCCAGGACAG |
| SEQ ID: 61 | 285-60 | GATCTGTGTAGTCAGGC |
| SEQ ID: 62 | 285-61 | GATCCCCACACACCCAT |
| SEQ ID: 63 | 285-62 | GATCAGGTGGGAAGGTA |
| SEQ ID: 64 | 285-63 | GATCAGTTATCAGGCAA |
| SEQ ID: 65 | 285-64 | GATCCCCCTTCATGTGA |
| SEQ ID: 66 | 285-65 | GATCCGACACGCGATAC |
| SEQ ID: 67 | 285-66 | GATCCCAGGCCCCAGGG |
| SEQ ID: 68 | 285-67 | GATCATAATCAGAGGGC |
| SEQ ID: 69 | 285-68 | GATCCTAATGAACCACT |
| SEQ ID: 70 | 285-69 | GATCCTGGAACCGAAAG |
| SEQ ID: 71 | 285-70 | GATCCCGAGTCCTCGCG |
| SEQ ID: 72 | 285-71 | GATCCACTCAAGAACAG |
| SEQ ID: 73 | 285-72 | GATCGTAGATGAATTTC |
| SEQ ID: 74 | 285-73 | GATCGTTCCATAACATG |
| SEQ ID: 75 | 285-74 | GATCATTAACAACCAGG |
| SEQ ID: 76 | 285-75 | GATCTCAATGGGCTTTT |
| SEQ ID: 77 | 285-76 | GATCACAGAGGCTCTCC |
| SEQ ID: 78 | 285-77 | GATCACCTTTCTAAATA |
| SEQ ID: 79 | 285-78 | GATCCTCGGATGTCATG |
| SEQ ID: 80 | 285-79 | GATCCAACAGATGGCCA |
| SEQ ID: 81 | 285-80 | GATCCGAGTACGGAAGA |
| SEQ ID: 82 | 285-81 | GATCTGCCTTTATTTTG |
| SEQ ID: 83 | 285-82 | GATCCCCTCCCGCCCTT |
| SEQ ID: 84 | 285-83 | GATCCAACTAATTCATT |
| SEQ ID: 85 | 285-84 | GATCCCCACATTCTTTA |
| SEQ ID: 86 | 285-85 | GATCCAAGACAAAGCCA |
| SEQ ID: 87 | 285-86 | GATCAGTTTTCTGTGTT |
| SEQ ID: 88 | 285-87 | GATCATACACATTTCCT |
| SEQ ID: 89 | 285-88 | GATCCCCTTGGGTACTG |
| SEQ ID: 90 | 285-89 | GATCCAATTTTTGTAGT |
| SEQ ID: 91 | 285-90 | GATCGTTCCTATGTGTC |
| SEQ ID: 92 | 285-91 | GATCGCATGTTTTGTTA |
| SEQ ID: 93 | 285-92 | GATCCTGTCTACCTACT |
| SEQ ID: 94 | 285-93 | GATCCGCCTGCCGCTCA |
| SEQ ID: 95 | 285-94 | GATCTCTCTATGTTGAA |
| SEQ ID: 96 | 285-95 | GATCTGTACAAAGTGGT |
| SEQ ID: 97 | 285-96 | GATCATTACGTGTTTAT |
| SEQ ID: 98 | 285-97 | GATCATTTTATAATTTC |
| SEQ ID: 99 | 285-98 | GATCTGCTCCTTCTCCT |
| SEQ ID: 100 | 285-99 | GATCCCGGGAGCGGTGC |
| SEQ ID: 101 | 285-100 | GATCATCATAGTTTTTA |
| SEQ ID: 102 | 285-101 | GATCCTGACACCTGCAG |
| SEQ ID: 103 | 285-102 | GATCCGCGGGCCGAGGG |
| SEQ ID: 104 | 285-103 | GATCCTTCTGCCTCTGT |
| SEQ ID: 105 | 285-104 | GATCTCCTGTGCTCAAA |
| SEQ ID: 106 | 285-105 | GATCTCTCTATGGCCAC |
| SEQ ID: 107 | 285-106 | GATCATCCTTTTTGTCT |
| SEQ ID: 108 | 285-107 | GATCACTGCATTTCCTG |
| SEQ ID: 109 | 285-108 | GATCACTGGAGGGCTAG |
| SEQ ID: 110 | 285-109 | GATCCCCAACCCCCCAA |
| SEQ ID: 111 | 285-110 | GATCCCCAGCCACCCAT |
| SEQ ID: 112 | 285-111 | GATCATTAGGAGAACTG |
| SEQ ID: 113 | 285-112 | GATCGCCAACACATTCG |
| SEQ ID: 114 | 285-113 | GATCCCCTCACCCCCAC |
| SEQ ID: 115 | 285-114 | GATCCTTTTCCTGTTGA |
| SEQ ID: 116 | 285-115 | GATCATTGTTATCATTT |
| SEQ ID: 117 | 285-116 | GATCATTTCATCTATAA |
| SEQ ID: 118 | 285-117 | GATCCCTAGCTGTGCTC |
| SEQ ID: 119 | 285-118 | GATCTAGCACGTCCGCC |
| SEQ ID: 120 | 285-119 | GATCCTGGGGACCCCTA |
| SEQ ID: 121 | 285-120 | GATCTCCTCTTTCATCA |
| SEQ ID: 122 | 285-121 | GATCTGTTTCATTGTGT |
| SEQ ID: 123 | 285-122 | GATCCGTTATGCCACTT |
| SEQ ID: 124 | 285-123 | GATCTCGTCGGTCAGCC |
| SEQ ID: 125 | 285-124 | GATCTGCTTGTCCGCGG |
| SEQ ID: 126 | 285-125 | GATCCGAGCAGCCTTGG |
| SEQ ID: 127 | 285-126 | GATCCTGTGGGGAAGCC |
| SEQ ID: 128 | 285-127 | GATCCTAGGCGTCCTCA |
| SEQ ID: 129 | 285-128 | GATCTGTTTACGGATGA |
| SEQ ID: 130 | 285-129 | GATCCTTTCGTAACTCC |
| SEQ ID: 131 | 285-130 | GATCCAAAGAGTCGCAG |
| SEQ ID: 132 | 285-131 | GATCGTCAGGATGAGTA |
| SEQ ID: 133 | 285-132 | GATCTCGTGGGGTTTCA |
| SEQ ID: 134 | 285-133 | GATCTGAGACTTTCCAT |
| SEQ ID: 135 | 285-134 | GATCCAGGGTCTGTATT |
| SEQ ID: 136 | 285-135 | GATCTTCAGTTATTTTG |
| SEQ ID: 137 | 285-136 | GATCTGCAGATGAATAC |
| SEQ ID: 138 | 285-137 | GATCTCACTCGGTCACC |
| SEQ ID: 139 | 285-138 | GATCCACAGTGTTCAAG |
| SEQ ID: 140 | 285-139 | GATCTTTTTCCTTTTTC |
| SEQ ID: 141 | 285-140 | GATCGCAGTGTGGAAAC |
| SEQ ID: 142 | 285-141 | GATCCAGAGGTGGGTGG |
| SEQ ID: 143 | 285-142 | GATCTGATGATTCCAAT |
| SEQ ID: 144 | 285-143 | GATCCTCCATGGTAGAG |
| SEQ ID: 145 | 285-144 | GATCCTGGGTTGTGCTT |
| SEQ ID: 146 | 285-145 | GATCCTGGGTTGTGCTT |
| SEQ ID: 147 | 285-146 | GATCCTTTTAAAACTGT |
| SEQ ID: 148 | 285-147 | GATCGTGTAATCTAAAA |
| SEQ ID: 149 | 285-148 | GATCGTTGAGGCCGCAG |
| SEQ ID: 150 | 285-149 | GATCTCCTTCACTTCTT |
| SEQ ID: 151 | 285-150 | GATCCAGGATGGCTGAC |
| SEQ ID: 152 | 285-151 | GATCCAGGGGGCATTTA |
| SEQ ID: 153 | 285-152 | GATCCAGGTCTTCAACC |
| SEQ ID: 154 | 285-153 | GATCCAGGTGCTGGAGG |
| SEQ ID: 155 | 285-154 | GATCCATCTCCACAGCC |
| SEQ ID: 156 | 285-155 | GATCCCCACACCAAGCA |
| SEQ ID: 157 | 285-156 | GATCCAGTCATCCAGGC |
| SEQ ID: 158 | 285-157 | GATCCCAGCACCTCCCA |
| SEQ ID: 159 | 285-158 | GATCCCCGGGGGAGAGG |
| SEQ ID: 160 | 285-159 | GATCTCCACAGGAACTG |
| SEQ ID: 161 | 285-160 | GATCCCGTGAATTACGA |
| SEQ ID: 162 | 285-161 | GATCCGGGGAATCACCA |
| SEQ ID: 163 | 285-162 | GATCCTAAGTCAATTAC |
| SEQ ID: 164 | 285-163 | GATCTCTCTAACCTCCC |
| SEQ ID: 165 | 285-164 | GATCCTCACAAAAGTGC |
| SEQ ID: 166 | 285-165 | GATCCTGAGAATGATTT |
| SEQ ID: 167 | 285-166 | GATCCTGGGTGTAAATG |
| SEQ ID: 168 | 285-167 | GATCCTTAGGACACTGT |
| SEQ ID: 169 | 285-168 | GATCCTTGAATTTACTT |
| SEQ ID: 170 | 285-169 | GATCGCCAACCCATTCG |
| SEQ ID: 171 | 285-170 | GATCGCCCCCTACCGCC |
| SEQ ID: 172 | 285-171 | GATCGCTCCACCTGGCT |
| SEQ ID: 173 | 285-172 | GATCCCTTCTCACTGTA |
| SEQ ID: 174 | 285-173 | GATCCCCCGCCCTTCAC |
| SEQ ID: 175 | 285-174 | GATCTAAGAGTTGTAGG |
| SEQ ID: 176 | 285-175 | GATCCTAGTGAAGGAAA |
| SEQ ID: 177 | 285-176 | GATCCTCAAATACAGCA |
| SEQ ID: 178 | 285-177 | GATCCTCTCTTTGGTCT |
| SEQ ID: 179 | 285-178 | GATCTGAAAAATCTCCT |
| SEQ ID: 180 | 285-179 | GATCCGCTGCCGCACCT |
| SEQ ID: 181 | 285-180 | GATCCTGTGACAGAGTG |
| SEQ ID: 182 | 285-181 | GATCGCCACTTACCTCC |
| SEQ ID: 183 | 285-182 | GATCTGGATGTGCTTTA |
| SEQ ID: 184 | 285-183 | GATCGGAGTTTATCGAT |
| SEQ ID: 185 | 285-184 | GATCGGCTTACACAACG |
| SEQ ID: 186 | 285-185 | GATCGTTTTTATTGCCA |
| SEQ ID: 187 | 285-186 | GATCTAAAAAAGTAACT |
| SEQ ID: 188 | 285-187 | GATCTAACTGGTCCTGG |
| SEQ ID: 189 | 285-188 | GATCCGCAGGGTCCGCA |
| SEQ ID: 190 | 285-189 | GATCCGGGGTTTGTCTA |
| SEQ ID: 191 | 285-190 | GATCCTGACCAGCATTT |
| SEQ ID: 192 | 285-191 | GATCTCCGTGGCCTCAC |
| SEQ ID: 193 | 285-192 | GATCTAATCTAATACCA |
| SEQ ID: 194 | 285-193 | GATCTCTCTGCCCTGCA |
| SEQ ID: 195 | 285-194 | GATCTCTTTCTTATAAA |
| SEQ ID: 196 | 285-195 | GATCTGACAAAAGTGGG |
| SEQ ID: 197 | 285-196 | GATCTGGTCACTGTTCC |
| SEQ ID: 198 | 285-197 | GATCCTGGCTGCTTCTT |
| SEQ ID: 199 | 285-198 | GATCTGTTGTATGATTT |
| SEQ ID: 200 | 285-199 | GATCCTCACCCACCCAA |
| SEQ ID: 201 | 285-200 | GATCTCAACATTAGTGA |
| SEQ ID: 202 | 285-201 | GATCTGGTTGGAATGTT |
| SEQ ID: 203 | 285-202 | GATCCTGACCATCCTAG |
| SEQ ID: 204 | 285-203 | GATCCTGTTCTATGAAG |
| SEQ ID: 205 | 285-204 | GATCGCAGCGTATTCTG |
| SEQ ID: 206 | 285-205 | GATCTCCAAAGGAATGG |
| SEQ ID: 207 | 285-206 | GATCTCCAAAGGAATGG |
| SEQ ID: 208 | 285-207 | GATCCCTTGGGGTGTCT |
| SEQ ID: 209 | 285-208 | GATCTACCCGCCTCTAC |
| SEQ ID: 210 | 285-209 | GATCGAGCGCACAGTAG |
| SEQ ID: 211 | 285-210 | GATCGCACGTTTTTTTA |
| SEQ ID: 212 | 285-211 | GATCGTAAACATAATTA |
| SEQ ID: 213 | 285-212 | GATCTGATGGAAATACA |
| SEQ ID: 214 | 285-213 | GATCGTCCTCAGCCGCA |
| SEQ ID: 215 | 285-214 | GATCTAAACCCAGGCAG |
| SEQ ID: 216 | 285-215 | GATCGCCTCCTACCGCC |
| SEQ ID: 217 | 285-216 | GATCGCTCCCAGGGCTC |
| SEQ ID: 218 | 285-217 | GATCGGTTTGCAGATGA |
| SEQ ID: 219 | 285-218 | GATCTTAATGTGCCCGT |
| SEQ ID: 220 | 285-219 | GATCTCCACCTTCTGAT |
| SEQ ID: 221 | 285-220 | GATCTCCTTGTGGGAAT |
| SEQ ID: 222 | 285-221 | GATCTAGCCTCAATGGA |
| SEQ ID: 223 | 285-222 | GATCTTCCTATGAACTA |
| SEQ ID: 224 | 285-223 | GATCTCCTGCCGGTGCT |
| SEQ ID: 225 | 285-224 | GATCTCGGCCTTGTGCT |
| SEQ ID: 226 | 285-225 | GATCTGACATTGCTCAT |
| SEQ ID: 227 | 285-226 | GATCTTTGCTAAATGAG |
| SEQ ID: 228 | 285-227 | GATCTCTTCAGTGTTGC |
| SEQ ID: 229 | 285-228 | GATCTGTGTGGACTGGC |
| SEQ ID: 230 | 285-229 | GATCTCGGCTTGCTGCA |
| SEQ ID: 231 | 285-230 | GATCTGGGCCAGTTTCT |
| SEQ ID: 232 | 285-231 | GATCTGAAGCTCAGTTT |
| SEQ ID: 233 | 285-232 | GATCTGGGCGGCGACCC |
| SEQ ID: 234 | 285-233 | GATCTGTGCCAGCAACT |
| SEQ ID: 235 | 285-234 | GATCTGTCGGAGCAACT |
| SEQ ID: 236 | 285-235 | GATCTGTTTCAAAGTTG |
| SEQ ID: 237 | 285-236 | GATCTTATAAGCTCTGC |
| SEQ ID: 238 | 285-237 | GATCTTATTTTCAAAGA |
| SEQ ID: 239 | 285-238 | GATCTTCAAGTTCTGAA |
| SEQ ID: 240 | 285-239 | GATCTTCTCCAAGAACT |
| SEQ ID: 241 | 285-240 | GATCTCAAAGAAACAAG |
| SEQ ID: 242 | 285-241 | GATCTTCTTCTCCTTTG |
| SEQ ID: 243 | 285-242 | GATCTCAGGAGTGTCCC |
| SEQ ID: 244 | 285-243 | GATCTCCCTGAAGGCCA |
| SEQ ID: 245 | 285-244 | GATCTTTCTTCTCCTTT |
| SEQ ID: 246 | 285-245 | GATCTTTGTTCATTTTC |
| SEQ ID: 247 | 285-246 | GATCTGCCCAGAGTTAT |
| SEQ ID: 248 | 285-247 | GATCTGCGGTACAGACT |
| SEQ ID: 249 | 285-248 | GATCTGCTACAAACCAA |
| SEQ ID: 250 | 285-249 | GATCTGGAAGGAATCGG |
| SEQ ID: 251 | 285-250 | GATCTGGCTCATGGGGA |
| SEQ ID: 252 | 285-251 | GATCTGTTTTCTGCAGG |
| SEQ ID: 253 | 285-252 | GATCTTCAGGGTGGTTA |
| SEQ ID: 254 | 285-253 | GATCTGAACTAATATAA |
| SEQ ID: 255 | 285-254 | GATCAGTGAGTGAAGGA |
| SEQ ID: 256 | 285-255 | GATCAGGTTGTGTCCTC |
| SEQ ID: 257 | 285-256 | GATCAGGTTGTGTCCTC |
| SEQ ID: 258 | 285-257 | GATCTCTCCTTTTCTTC |
| SEQ ID: 259 | 285-258 | GATCTCTTGTATTCCTA |
| SEQ ID: 260 | 285-259 | GATCCCCTCCTGCCCTC |
| SEQ ID: 261 | 285-260 | GATCTATGTATCAAAAA |
| SEQ ID: 262 | 285-261 | GATCATGCCACTACTGC |
| SEQ ID: 263 | 285-262 | GATCCACAGACCACCTT |
| SEQ ID: 264 | 285-263 | GATCTACTGTCATGTGT |
| SEQ ID: 265 | 285-264 | GATCCCCTCCAGCCCTC |
| SEQ ID: 266 | 285-265 | GATCAAAGAGAGAAGGC |
| SEQ ID: 267 | 285-266 | GATCCCCAAACAGTTTC |
| SEQ ID: 268 | 285-267 | GATCCTTAATGTTGTGA |
| SEQ ID: 269 | 285-268 | GATCTGGAGCATGATGG |
| SEQ ID: 270 | 285-269 | GATCAAGACCCTCAACA |
| SEQ ID: 271 | 285-270 | GATCAAGAATCTTGTGT |
| SEQ ID: 272 | 285-271 | GATCCTCCTGGCCCGCC |
| SEQ ID: 273 | 285-272 | GATCACATTTCATATCA |
| SEQ ID: 274 | 285-273 | GATCTGAACCAGAAGCT |
| SEQ ID: 275 | 285-274 | GATCAATCCACTCGTGA |
| SEQ ID: 276 | 285-275 | GATCCCCAGCCACCCAG |
| SEQ ID: 277 | 285-276 | GATCTGGAGCTAGACGG |
| SEQ ID: 278 | 285-277 | GATCACGCGGTCAGGAG |
| SEQ ID: 279 | 285-278 | GATCCGCACCCACCCAC |
| SEQ ID: 280 | 285-279 | GATCTGCTACTAACCAA |
| SEQ ID: 281 | 285-280 | GATCGCCACCTACCTCC |
| SEQ ID: 282 | 285-281 | GATCAGTGTGTGTAGAA |
| SEQ ID: 283 | 285-282 | GATCCGCTGCATATTCA |
| SEQ ID: 284 | 285-283 | GATCCCCTGCCGCCCTC |
| SEQ ID: 285 | 285-284 | GATCTCCACATTCATCT |
| SEQ ID: 286 | 285-285 | GATCTTAAAAATCACCC |
| SEQ ID: 287 | 205-1 | GATCAAAACCCAGCAGA |
| SEQ ID: 288 | 205-2 | GATCAAAATGAAACCTG |
| SEQ ID: 289 | 205-3 | GATCAAACCAAGGCCCA |
| SEQ ID: 290 | 205-4 | GATCAAACTCCCCACCC |
| SEQ ID: 291 | 205-5 | GATCAAACTCCCCACCC |
| SEQ ID: 292 | 205-6 | GATCAAAGAAAGAAGGC |
| SEQ ID: 293 | 205-7 | GATCAAAGACATCCTCA |
| SEQ ID: 294 | 205-8 | GATCAAATAAAGTTATA |
| SEQ ID: 295 | 205-9 | GATCAAATGTGTGGCCT |
| SEQ ID: 296 | 205-10 | GATCAAATTTGAACTTC |
| SEQ ID: 297 | 205-11 | GATCAACAGGCTTACAG |
| SEQ ID: 298 | 205-12 | GATCAACCATCGCTTTA |
| SEQ ID: 299 | 205-13 | GATCAACTGAACCAGTA |
| SEQ ID: 300 | 205-14 | GATCAAGCCTTTCTTTC |
| SEQ ID: 301 | 205-15 | GATCAAGCGTGCTTTCC |
| SEQ ID: 302 | 205-16 | GATCAAGTTCCCGCTGC |
| SEQ ID: 303 | 205-17 | GATCAAGTTTAAATGAC |
| SEQ ID: 304 | 205-18 | GATCAATAAAGTCAGTG |
| SEQ ID: 305 | 205-19 | GATCAATAATAATGAGG |
| SEQ ID: 306 | 205-20 | GATCAATGAAGTGAGAA |
| SEQ ID: 307 | 205-21 | GATCAATGACAGAGCCT |
| SEQ ID: 308 | 205-22 | GATCAATGCCCTCATTA |
| SEQ ID: 309 | 205-23 | GATCACACCACTGCACT |
| SEQ ID: 310 | 205-24 | GATCACAGCCGAAGGAG |
| SEQ ID: 311 | 205-25 | GATCACATAAAACAGAT |
| SEQ ID: 312 | 205-26 | GATCACATTTTCTGTTG |
| SEQ ID: 313 | 205-27 | GATCACCAAACCAGTCC |
| SEQ ID: 314 | 205-28 | GATCACCCATTCCGGGT |
| SEQ ID: 315 | 205-29 | GATCACCCCCTCCCCAA |
| SEQ ID: 316 | 205-30 | GATCACCTCTGAGACCC |
| SEQ ID: 317 | 205-31 | GATCACCTGAGGTCAGG |
| SEQ ID: 318 | 205-32 | GATCACCTGAGGTCGGG |
| SEQ ID: 319 | 205-33 | GATCACCTTGGTGTTCC |
| SEQ ID: 320 | 205-34 | GATCACGCCACTGCACT |
| SEQ ID: 321 | 205-35 | GATCACTGCAGCTTCTA |
| SEQ ID: 322 | 205-36 | GATCACTTAGCAACATG |
| SEQ ID: 323 | 205-37 | GATCACTTGAGCCCAGG |
| SEQ ID: 324 | 205-38 | GATCACTTGAGGTCAGG |
| SEQ ID: 325 | 205-39 | GATCAGAACCTCCAAAT |
| SEQ ID: 326 | 205-40 | GATCAGAATCATGGTCT |
| SEQ ID: 327 | 205-41 | GATCAGCATTGTGACTT |
| SEQ ID: 328 | 205-42 | GATCAGCCCCACCCTGG |
| SEQ ID: 329 | 205-43 | GATCAGCGCTTTACAAA |
| SEQ ID: 330 | 205-44 | GATCAGGACACTTAGCA |
| SEQ ID: 331 | 205-45 | GATCAGTGTTGAAGAAA |
| SEQ ID: 332 | 205-46 | GATCAGTTTGGGAAATG |
| SEQ ID: 333 | 205-47 | GATCAGTTTTTTCACCT |
| SEQ ID: 334 | 205-48 | GATCATAAATATTAATG |
| SEQ ID: 335 | 205-49 | GATCATCAAACTGATAA |
| SEQ ID: 336 | 205-50 | GATCATCCCTTTGGTTA |
| SEQ ID: 337 | 205-51 | GATCATCCTTCCTGGCA |
| SEQ ID: 338 | 205-52 | GATCATCCTTCCTGGCA |
| SEQ ID: 339 | 205-53 | GATCATCTAAACTGAGT |
| SEQ ID: 340 | 205-54 | GATCATGCATTGTTGAG |
| SEQ ID: 341 | 205-55 | GATCATGCTGCCCTGGG |
| SEQ ID: 342 | 205-56 | GATCATGTAGCTGAGAC |
| SEQ ID: 343 | 205-57 | GATCATGTCTTTTCCAT |
| SEQ ID: 344 | 205-58 | GATCATGTTATGATTTG |
| SEQ ID: 345 | 205-59 | GATCATTATTTGGAAAT |
| SEQ ID: 346 | 205-60 | GATCATTCCTTCTGTAG |
| SEQ ID: 347 | 205-61 | GATCATTGTTGAACTTC |
| SEQ ID: 348 | 205-62 | GATCATTTCATATTGCT |
| SEQ ID: 349 | 205-63 | GATCATTTGACAACTGG |
| SEQ ID: 350 | 205-64 | GATCATTTTGGAACCAG |
| SEQ ID: 351 | 205-65 | GATCCAAGTGTTTAACC |
| SEQ ID: 352 | 205-66 | GATCCAATGGAGCCTGG |
| SEQ ID: 353 | 205-67 | GATCCACACGTTCCGGG |
| SEQ ID: 354 | 205-68 | GATCCACATCTCAAAGA |
| SEQ ID: 355 | 205-69 | GATCCACTACCGGAAGA |
| SEQ ID: 356 | 205-70 | GATCCACTAGACAGTTT |
| SEQ ID: 357 | 205-71 | GATCCACTTCTGTGATT |
| SEQ ID: 358 | 205-72 | GATCCAGGGTGTGTGTG |
| SEQ ID: 359 | 205-73 | GATCCAGGTGACTCTGA |
| SEQ ID: 360 | 205-74 | GATCCAGTGTCCATGGA |
| SEQ ID: 361 | 205-75 | GATCCATCGTGATGTCT |
| SEQ ID: 362 | 205-76 | GATCCATCTGCCTTTGT |
| SEQ ID: 363 | 205-77 | GATCCATGAAGGAATCG |
| SEQ ID: 364 | 205-78 | GATCCATGAAGTCATTC |
| SEQ ID: 365 | 205-79 | GATCCATTTCATAAAGT |
| SEQ ID: 366 | 205-80 | GATCCCAATGCACCCAA |
| SEQ ID: 367 | 205-81 | GATCCCAGACTGGTTCA |
| SEQ ID: 368 | 205-82 | GATCCCAGACTGGTTCC |
| SEQ ID: 369 | 205-83 | GATCCCAGCAAGATAAT |
| SEQ ID: 370 | 205-84 | GATCCCAGGGAAATATC |
| SEQ ID: 371 | 205-85 | GATCCCAGGGGCTTATC |
| SEQ ID: 372 | 205-86 | GATCCCAGGGGCTTATC |
| SEQ ID: 373 | 205-87 | GATCCCAGTCTCTGCCA |
| SEQ ID: 374 | 205-88 | GATCCCATTTAATATTT |
| SEQ ID: 375 | 205-89 | GATCCCCACCCACCCAC |
| SEQ ID: 376 | 205-90 | GATCCCCCCACAGCCCC |
| SEQ ID: 377 | 205-91 | GATCCCCGGTGGTTTTG |
| SEQ ID: 378 | 205-92 | GATCCCCTCAGAAGGCA |
| SEQ ID: 379 | 205-93 | GATCCCCTCCCTCCCTC |
| SEQ ID: 380 | 205-94 | GATCCCCTCTGAGTCCT |
| SEQ ID: 381 | 205-95 | GATCCCCTGCCTGGTGC |
| SEQ ID: 382 | 205-96 | GATCCCGCAGGTGGCAC |
| SEQ ID: 383 | 205-97 | GATCCCGCAGGTGGCAC |
| SEQ ID: 384 | 205-98 | GATCCCTAACTGTTCCC |
| SEQ ID: 385 | 205-99 | GATCCCTACTGTTTTCT |
| SEQ ID: 386 | 205-100 | GATCCCTCCCTTACCAC |
| SEQ ID: 387 | 205-101 | GATCCCTCCCTTACCAT |
| SEQ ID: 388 | 205-102 | GATCCCTTTTTTATTTT |
| SEQ ID: 389 | 205-103 | GATCCGAGGAGGCGGAA |
| SEQ ID: 390 | 205-104 | GATCCGCAAGAACAAGT |
| SEQ ID: 391 | 205-105 | GATCCGCCCACCTCGGC |
| SEQ ID: 392 | 205-106 | GATCCGCCCTCGAATGG |
| SEQ ID: 393 | 205-107 | GATCCGGAAATATGGCC |
| SEQ ID: 394 | 205-108 | GATCCGGATTGGTGCCA |
| SEQ ID: 395 | 205-109 | GATCCGGTCTCTCTGCG |
| SEQ ID: 396 | 205-110 | GATCCGGTCTCTGTGCG |
| SEQ ID: 397 | 205-111 | GATCCGTCCCTAACAAA |
| SEQ ID: 398 | 205-112 | GATCCGTCCCTAACAAC |
| SEQ ID: 399 | 205-113 | GATCCGTTCCGTGGTCG |
| SEQ ID: 400 | 205-114 | GATCCTAAGAGGAAAGT |
| SEQ ID: 401 | 205-115 | GATCCTAAGCCATAGAC |
| SEQ ID: 402 | 205-116 | GATCCTACTCTCTTATC |
| SEQ ID: 403 | 205-117 | GATCCTACTCTCTTATT |
| SEQ ID: 404 | 205-118 | GATCCTACTGATGAAAT |
| SEQ ID: 405 | 205-119 | GATCCTCAACCAATAAA |
| SEQ ID: 406 | 205-120 | GATCCTCAGAACTTCTC |
| SEQ ID: 407 | 205-121 | GATCCTCAGCCTCCCAG |
| SEQ ID: 408 | 205-122 | GATCCTGAAGAGATTGA |
| SEQ ID: 409 | 205-123 | GATCCTGACACTAAGGC |
| SEQ ID: 410 | 205-124 | GATCCTGATGCTGCCAG |
| SEQ ID: 411 | 205-125 | GATCCTGCTAAATATTA |
| SEQ ID: 412 | 205-126 | GATCCTGCTGCTGTAAT |
| SEQ ID: 413 | 205-127 | GATCCTGCTGGAAACCA |
| SEQ ID: 414 | 205-128 | GATCCTGGCCATGAATG |
| SEQ ID: 415 | 205-129 | GATCCTGGGGCAACCCA |
| SEQ ID: 416 | 205-130 | GATCCTGTAGTGTTCCT |
| SEQ ID: 417 | 205-131 | GATCCTTACGGAAAAGG |
| SEQ ID: 418 | 205-132 | GATCCTTGACGAGGAGA |
| SEQ ID: 419 | 205-133 | GATCCTTTTATCCTGCT |
| SEQ ID: 420 | 205-134 | GATCGAACATTTCACCT |
| SEQ ID: 421 | 205-135 | GATCGCACCACTGCGTT |
| SEQ ID: 422 | 205-136 | GATCGCAGTTTGGAAAC |
| SEQ ID: 423 | 205-137 | GATCGCCGTTCTGGTAA |
| SEQ ID: 424 | 205-138 | GATCGCGCCACTGCACT |
| SEQ ID: 425 | 205-139 | GATCGCTCACAATGTTT |
| SEQ ID: 426 | 205-140 | GATCGCTCCAATAAACA |
| SEQ ID: 427 | 205-141 | GATCGCTTTCTACACTG |
| SEQ ID: 428 | 205-142 | GATCGGCCACTACCTGG |
| SEQ ID: 429 | 205-143 | GATCGTTAATGCCCTAA |
| SEQ ID: 430 | 205-144 | GATCGTTAATGCCCTTG |
| SEQ ID: 431 | 205-145 | GATCGTTCTTGATTTTG |
| SEQ ID: 432 | 205-146 | GATCGTTTCCAGATGAG |
| SEQ ID: 433 | 205-147 | GATCTAAAGAAATAAAG |
| SEQ ID: 434 | 205-148 | GATCTAAAGATTTCTCT |
| SEQ ID: 435 | 205-149 | GATCTAAGAGTTACCTG |
| SEQ ID: 436 | 205-150 | GATCTAAGCAGCAGGGA |
| SEQ ID: 437 | 205-151 | GATCTAAGTTGCCTACC |
| SEQ ID: 438 | 205-152 | GATCTAATCTGTGCTAC |
| SEQ ID: 439 | 205-153 | GATCTAATGTAAAATCC |
| SEQ ID: 440 | 205-154 | GATCTAATTAAACTAAA |
| SEQ ID: 441 | 205-155 | GATCTACAATGAAGCCC |
| SEQ ID: 442 | 205-156 | GATCTACATACAAACAA |
| SEQ ID: 443 | 205-157 | GATCTAGAAATTGCCCT |
| SEQ ID: 444 | 205-158 | GATCTATCACCTGTCAT |
| SEQ ID: 445 | 205-159 | GATCTATTGAGAGCCCT |
| SEQ ID: 446 | 205-160 | GATCTATTTTTTCTAAA |
| SEQ ID: 447 | 205-161 | GATCTCAAGAGCCCTGT |
| SEQ ID: 448 | 205-162 | GATCTCAATGCCAATCC |
| SEQ ID: 449 | 205-163 | GATCTCAGTTTCCTGGC |
| SEQ ID: 450 | 205-164 | GATCTCCAACCAGGCCA |
| SEQ ID: 451 | 205-165 | GATCTCCGAGTCAGGAC |
| SEQ ID: 452 | 205-166 | GATCTCTGGCAGTGGAG |
| SEQ ID: 453 | 205-167 | GATCTCTTTTTATTTAA |
| SEQ ID: 454 | 205-168 | GATCTGAATAGAGAAAT |
| SEQ ID: 455 | 205-169 | GATCTGACTGCCTCCTC |
| SEQ ID: 456 | 205-170 | GATCTGAGTTCAGAAGG |
| SEQ ID: 457 | 205-171 | GATCTGAGTTCGAGACC |
| SEQ ID: 458 | 205-172 | GATCTGAGTTCAGACCG |
| SEQ ID: 459 | 205-173 | GATCTGAGTTCAGACCT |
| SEQ ID: 460 | 205-174 | GATCTGAGTTCAGACGT |
| SEQ ID: 461 | 205-175 | GATCTGATGTTCCTTTT |
| SEQ ID: 462 | 205-176 | GATCTGATTATTTACTT |
| SEQ ID: 463 | 205-177 | GATCTGCAGATGAAGAC |
| SEQ ID: 464 | 205-178 | GATCTGCATATTTCTGT |
| SEQ ID: 465 | 205-179 | GATCTGCGTGTGTCCAG |
| SEQ ID: 466 | 205-180 | GATCTGCTTCTCCAGTT |
| SEQ ID: 467 | 205-181 | GATCTGCTTCTCCAGTT |
| SEQ ID: 468 | 205-182 | GATCTGGAAGATGAGTG |
| SEQ ID: 469 | 205-183 | GATCTGGATTACTATGT |
| SEQ ID: 470 | 205-184 | GATCTGGCCGTCAGCCG |
| SEQ ID: 471 | 205-185 | GATCTGGCTGAACCAGT |
| SEQ ID: 472 | 205-186 | GATCTGGTATTAGGAAA |
| SEQ ID: 473 | 205-187 | GATCTGGTCTAGTTAAC |
| SEQ ID: 474 | 205-188 | GATCTGTAATAGCATAT |
| SEQ ID: 475 | 205-189 | GATCTGTATCAAGATAA |
| SEQ ID: 476 | 205-190 | GATCTGTGGAGAATGTA |
| SEQ ID: 477 | 205-191 | GATCTGTGGAGAATTTA |
| SEQ ID: 478 | 205-192 | GATCTGTGTTTGCTCTG |
| SEQ ID: 479 | 205-193 | GATCTGTTCAGTGTCAC |
| SEQ ID: 480 | 205-194 | GATCTTAATATATTTGA |
| SEQ ID: 481 | 205-195 | GATCTTATTTGGAATTG |
| SEQ ID: 482 | 205-196 | GATCTTCAAAGGTGGGC |
| SEQ ID: 483 | 205-197 | GATCTTCAAGTGAACAT |
| SEQ ID: 484 | 205-198 | GATCTTCCCATTCACAG |
| SEQ ID: 485 | 205-199 | GATCTTCTGTAAATGGA |
| SEQ ID: 486 | 205-200 | GATCTTCTGTGGTGCTT |
| SEQ ID: 487 | 205-201 | GATCTTCTTTATAATTC |
| SEQ ID: 488 | 205-202 | GATCTTGTTTTTATTGT |
| SEQ ID: 489 | 205-203 | GATCTTTGCTGGCAAGC |
| SEQ ID: 490 | 205-204 | GATCTTTGTACGTAATT |
| SEQ ID: 491 | 205-205 | GATCTTTTCAGAGTGGT |
| SEQ ID: 492 | 59-4 AA | MSGQWFYEAKAKRHRDKIHGADIIRADMRKKRPQIAAEQSKDRENGAKES |
| | | WVNNVNKDAFLPPELAGVVEEPEEDAAPASPSSSVVBPASSVIDMSQENT |
| | | RKPNVSPEKRKNPFNSSKLPEGHSSQQTKNEQSKNGRTGLFQTSKEDELS |
| | | ESKEKSTVADTSIQKLEKSKQTLPGLSNGSQIKAPIPKARKMIYKSTDLN |
| | | KDDNQSFPRQRTDSLKARGAPRGILKRNSSSSSTDSETLRYNHNFEPKSK |
| | | IVSPGLTIHERISEKEHSLEDNSSENSLEPLKHVRFSAVKDELPQSPGLI |
| | | HGREVGEFSVLESDRLKNGMEDAGDTEEFQSDPKPSQYRKPSLFHQSTSS |
| | | PYVSKSETHQPMTSGSFPINGLHSHSEVLTARPQSMENSPTINEPKDKSS |
| | | ELTRLESVLPRSPADELSHCVEPEPSQVPGGSSRDRQQGSEEEPSPVLKT |
| | | LERSAARKMPSKSLEDISSDSSNQAKVDNQPEELVRSAEDDEKPDQKPVT |
| | | NECVPRISTVPTQPDNPFSHPDKLKRMSKSVPAFLQDEVSGSVMSVYSGD |
| | | FGNLEVKGNIQFAIEYVESLKELHVFVAQCKDLAAADVKKQRSDPYVKAY |
| | | LLPDKGKMGKKKTLVVKKTLNPVYNEILRYKIEKQILKTQKLNLSIWHRD |
| | | TFKRNSFLGEVELDLETWDWDNKQNKQLRWYPLKRKTAPVALEAENRGEM |
| | | KLAQYVPEPVPGKKLPTTGEVHIWVKECLDLPLLRGSHLNSFVKCTILP |
| | | DTSRKSRQKTRAVGKTTNPIFNHTMVYDGFRPEDLMEACVELTVWDHYKL |
| | | TNQFLGGLRIGFGTGKSYGTEVDWMDSTSEEVALWEKMVNSPNTWIEATL |
| | | PLRMLLIAKISK |

What is claimed is:

1. A composition comprising at least one expression vector, wherein the at least one expression vector comprises a nucleic acid comprising:
(a) at least one polynucleotide sequence selected from the group consisting of: SEQ ID NO: 1-SEQ ID NO: 491 or a polynucleotide sequence complementary thereto;
(b) at least one polynucleotide sequence comprising a conservative variation of a polynucleotide sequence of (a);
(c) at least one polynucleotide encoding a polypeptide sequence of SEQ ID NO: 492, or conservative variations thereof;
(d) at least one polynucleotide sequence that hybridizes under stringent conditions to a polynucleotide sequence of (a) or (b);
(e) at least one polynucleotide that is at least about 70% identical to a polynucleotide sequence of (a), or (b); or,
(f) at least one polynucleotide sequence comprising at least about 10 contiguous nucleotides of a polynucleotide sequence selected from the group consisting of: SEQ ID NO: 1-SEQ ID NO: 491, or a sequence complementary thereto.

2. The at least one expression vector of claim 1, wherein the at least one expression vector comprises a promoter operably linked to the nucleic acid comprising the polynucleotide of (a), (b), (c), (d), (e) or (f).

3. The at least one expression vector of claim 1, wherein the nucleic acid encodes a polypeptide.

4. The at least one expression vector of claim 1, wherein the nucleic acid encodes a sense or anti sense RNA.

5. A method of treating breast cancer in a patient, the method comprising administering to the patient an effective amount of the at least one expression vector of claim 1.

6. A composition comprising the at least one expression vector of claim 1 and an excipient.

7. The composition of claim 6, wherein the excipient is a pharmaceutically acceptable excipient.

8. A cell comprising the at least one expression vector of claim 1.

9. The cell of claim 8, which cell expresses a polypeptide of SEQ ID NO: 492.

10. An isolated or recombinant polypeptide comprising:
one or more amino acid sequences or subsequences encoded by a nucleic acid comprising:
(a) an amino acid sequence of SEQ ID NO: 492, and conservative variants thereof;
(b) an amino acid sequence encoded by a polynucleotide sequence selected from the group consisting of SEQ ID NO: 1 to SEQ ID NO: 491, and conservative variations thereof;
(c) an amino acid sequence encoded by a polynucleotide sequence that hybridizes under stringent conditions to a polynucleotide sequence selected from the group consisting of SEQ ID NO: 1 to SEQ ID NO: 491
(d) an amino acid sequence encoded by a polynucleotide sequence that is at least about 70% identical to a polynucleotide selected from the group consisting of SEQ ID NO: 1 to SEQ ID NO: 491; or
(e) a polypeptide comprising an amino acid subsequence of (a), (b), (c) or (d).

11. The isolated or recombinant polypeptide of claim 10, comprising a fusion protein.

12. The isolated or recombinant polypeptide of claim 10, comprising a peptide or polypeptide tag.

13. The isolated or recombinant polypeptide of claim 12, wherein the peptide or polypeptide tag comprises a reporter peptide or polypeptide.

14. The isolated or recombinant polypeptide of claim 12, wherein the peptide or polypeptide tag comprises an epitope.

15. The isolated or recombinant polypeptide of claim 12, wherein the peptide or polypeptide tag comprises a localization signal or sequence.

16. A composition comprising the isolated or recombinant polypeptide of claim 10 and an excipient.

17. The composition of claim 16, wherein the excipient is a pharmaceutically acceptable excipient.

18. A method of treating breast cancer in a patient, the method comprising administering to the patient an effective amount of the isolated or recombinant polypeptide of claim 10.

19. An array of polypeptides comprising two or more different polypeptides of claim 10.

20. An antibody specific for an isolated or recombinant polypeptide of claim 10.

21. The antibody of claim 20, wherein the antibody comprises a monoclonal antibody or polyclonal serum.

22. One or more isolated or recombinant polypeptides that bind to the antibody of claim 20.

23. The antibody of claim 20, which antibody is specific for an eptitope comprising a subsequence of a polypeptide of SEQ ID NO: 492.

24. A cell comprising at least one exogenous nucleic acid, which cell expresses a polypeptide of claim 10.

25. A labeled probe comprising a nucleic acid or polypeptide comprising:
(a) at least one polynucleotide sequence selected from the group consisting of: SEQ ID NO: 1-SEQ ID NO: 491 or a polynucleotide sequence complementary thereto;
(b) at least one polynucleotide sequence comprising a conservative variation of a polynucleotide sequence of (a);
(c) at least one polynucleotide encoding a polypeptide sequence of SEQ ID NO: 492, or conservative variations thereof;
(d) at least one polynucleotide sequence that hybridizes under stringent conditions to a polynucleotide sequence of (a) or (b);
(e) at least one polynucleotide that is at least about 70% identical to a polynucleotide sequence of (a), or (b);
(f) at least one polynucleotide sequence comprising at least about 10 contiguous nucleotides of a polynucleotide sequence selected from the group consisting of: SEQ ID NO: 1-SEQ ID NO: 491, or a sequence complementary thereto;
(g) at least one polypeptide or peptide comprising an amino acid sequence of SEQ ID NO: 492 or conservative variations thereof;
(h) at least one polypeptide or peptide comprising an amino acid subsequence of SEQ ID NO: 492, or conservative variants thereof comprising at least six amino acids; or,
(i) at least one antibody specific for a polypeptide or peptide sequence of (g) or (h).

26. The labeled probe of claim 25, comprising a nucleic acid.

27. The labeled probe of claim 25, comprising an oligonucleotide.

28. The labeled probe of claim 25, the polynucleotide sequence of (f) comprises at least about 12 contiguous nucleotides.

29. The labeled probe of claim 25, the polynucleotide sequence of (f) comprises at least about 14 contiguous nucleotides.

30. The labeled probe of claim 25, the polynucleotide sequence of (f) comprises at least about 16 contiguous nucleotides.

31. The labeled probe of claim 25, the polynucleotide sequence of (f) comprises at least about 17 contiguous nucleotides.

32. The labeled probe of claim 25, comprising a peptide.

33. The labeled probe of claim 25, comprising an antigenic peptide.

34. The labeled probe of claim 25, comprising a fusion protein.

35. The labeled probe of claim 25, comprising an epitope tag.

36. The labeled probe of claim 25, comprising an isotopic, fluorescent, fluorogenic or colorimetric label.

37. The labeled probe of claim 25, comprising a DNA or RNA molecule.

38. A labeled probe of claim 25, comprising a cDNA, an amplification product, a transcript, a restriction fragment or an oligonucleotide.

39. A labeled probe of claim 25, comprising an array of probes comprising a plurality of nucleic acids comprising:
(a) two or more polynucleotide sequences selected from SEQ ID NO: 1 to SEQ ID NO: 491, or a subsequence thereof comprising at least about 10 contiguous nucleotides of any of SEQ ID NO: 1 to SEQ ID NO: 491;
(b) two or more polynucleotide sequences complementary to the polynucleotide sequence of (a); or,
(c) two or more polynucleotide sequences that hybridize under stringent conditions to the polynucleotide sequences of (a) or (b).

40. An array of probes according to claim 39, wherein the nucleic acids are logically or physically arrayed.

41. A marker set for predicting at least one characteristic of a breast cancer cell, comprising a plurality of members, which members comprise:
(a) one or more polynucleotide sequence selected from the group consisting of: SEQ ID NO: 1-SEQ ID NO: 491 or a polynucleotide sequence complementary thereto;
(b) one or more polynucleotide sequence comprising a conservative variation of a polynucleotide sequence of (a);
(c) one or more polynucleotide encoding a polypeptide sequence of SEQ ID NO: 492, or conservative variations thereof;
(d) one or more polynucleotide sequence that hybridizes under stringent conditions to a polynucleotide sequence of (a) or (b);
(e) one or more polynucleotide that is at least about 70% identical to a polynucleotide sequence of (a) or (b);
(f) one or more polynucleotide sequence comprising at least about 10 contiguous nucleotides of a polynucleotide sequence selected from the group consisting of: SEQ ID NO: 1-SEQ ID NO: 491, or a sequence complementary thereto;
(g) one or more polypeptide comprising an amino acid sequence or subsequence of SEQ ID NO: 492 or conservative variants thereof comprising at least six amino acids; and/or,
(h) one or more antibodies specific for a polypeptide or peptide sequence of (g) or encoded by (c).

42. The marker set of claim 41, wherein the plurality of members comprise one or more of oligonucleotides, expression products, and amplification products.

43. The marker set of claim 42, wherein the oligonucleotides are synthetic oligonucleotides.

44. The marker set of claim 41, wherein the plurality of members comprise labeled nucleic acid probes.

45. The marker set of claim 41, wherein the plurality of members comprise a plurality of polypeptides or peptides.

46. The marker set of claim 41, wherein the plurality of members comprise a plurality of antibodies.

47. The marker set of claim 41, wherein the plurality of members, comprise nucleic acids and polypeptides.

48. The marker set of claim 41, wherein the plurality of members are logically or physically arrayed.

49. The marker set of claim 48, wherein the array comprises a bead array.

50. The marker set of claim 41, wherein each member of the marker set comprises at least about 10 contiguous nucleotides from at least one of SEQ ID NO: 1-SEQ ID NO: 491.

51. The marker set of claim 41, wherein the plurality of members together comprise a plurality of sequences or subsequences selected from a plurality of nucleic acids represented by SEQ ID NO: 1-SEQ ID NO: 491.

52. The marker set of claim 41, comprising a majority of members that together comprise a majority of subsequences from a majority of SEQ ID NO: 1-SEQ ID NO: 491.

53. The marker set of claim 41, wherein breast cancer is predicted by hybridizing the nucleic acids of the marker set to a DNA or RNA sample from a cell or a tissue, and detecting at least one expressed expression product.

54. An array comprising the marker set of claim 41.

55. The marker set of claim 41, wherein the at least one characteristic of a breast cancer cell is selected from the group consisting of transformation state, invasiveness, stage, a protein expressed, and a protein expressed on the surface of the breast cancer cell.

56. A method for modulating at least one characteristic of a breast cell, the method comprising:
modulating expression or activity of at least one polypeptide encoded by a nucleic acid, the nucleic acid comprising:
(a) at least one polynucleotide sequence selected from the group consisting of: SEQ ID NO: 1-SEQ ID NO: 491 or a sequence complementary thereto;
(b) at least one polynucleotide sequence comprising a conservative variation of a polynucleotide sequence of (a);
(c) at least one polynucleotide encoding a polypeptide sequence of SEQ ID NO: 492, or conservative variations thereof;
(d) at least one polynucleotide sequence that hybridizes under stringent conditions to a polynucleotide sequence of (a) or (b);
(e) at least one polynucleotide that is at least about 70% identical to a polynucleotide sequence of (a), or (b); or,
(f) at least one polynucleotide sequence comprising at least about 10 contiguous nucleotides of a polynucleotide sequence selected from the group consisting of: SEQ ID NO: 1-SEQ ID NO: 491, or a sequence complementary thereto.

57. The method of claim 56, comprising modulating expression by expressing an exogenous nucleic acid comprising a polynucleotide sequence selected from SEQ ID NO: 1 to SEQ ID NO: 491.

58. The method of claim 56, comprising modulating expression in a cell line or non-human mammal.

59. The method of claim 58, wherein the non-human mammal comprises a mouse, a rat, a dog, a rabbit, a pig, a sheep or a non-human primate.

60. The method of claim 56, comprising modulating expression by expressing an antisense RNA or a ribozyme.

61. The method of claim 56, wherein expression is modulated in response to a carcinogenic signal.

62. The method of claim 61, wherein a plurality of expression products are detected.

63. The method of claim 62, wherein the plurality of expression products are detected in an array.

64. The method of claim 63, wherein the array comprises a bead array.

65. The method of claim 63, wherein the array comprises a tissue array.

66. The method of claim 56, further comprising detecting altered expression or activity of an expression product encoded by a nucleic acid comprising a polynucleotide sequence selected from SEQ ID NO: 1 to SEQ ID NO: 491.

67. The method of claim 66, comprising detecting altered expression or activity in response to a carcinogenic signal.

68. The method of claim 66, wherein a data record comprising the altered expression or activity is recorded in a database.

69. The method of claim 68, wherein the database comprises a plurality of character strings recorded on a computer or in a computer readable medium.

70. The method of claim 56, wherein the at least one characteristic of a breast cell is selected from the group consisting of transformation state, invasiveness, stage, a protein expressed, and a protein expressed on the surface of the breast cell.

71. A method for identifying a breast cancer gene, the method comprising:
(i) providing at least one nucleic acid comprising:
(a) at least one polynucleotide sequence selected from the group consisting of: SEQ ID NO: 1-SEQ ID NO: 491, or a polynucleotide sequence complementary thereto;
(b) at least one polynucleotide encoding a polypeptide sequence of SEQ ID NO: 492, or conservative variations thereof;
(c) at least one polynucleotide sequence that hybridizes under stringent conditions to a polynucleotide sequence of (a) or (b);
(d) at least one polynucleotide that is at least about 70% identical to a polynucleotide sequence of (a), or (b);
(e) at least one polynucleotide sequence that hybridizes to a nucleic acid that is physically linked in the human genome to a nucleic acid comprising a polynucleotide sequence of (a), (b), (c) or (d); or,
(f) at least one polynucleotide sequence comprising at least about 10 contiguous nucleotides of a polynucleotide sequence selected from the group consisting of: SEQ ID NO: 1-SEQ ID NO: 491, or a sequence complementary thereto; and,
(ii) identifying at least one nucleic acid corresponding to a breast cancer gene.

72. The method of claim 71, wherein the polynucleotide sequence in (i) is selected from the group consisting of: SEQ ID NO: 1-SEQ ID NO: 491, or a conservative variation thereof.

73. The method of claim 72, wherein the breast cancer gene comprises a locus on human chromosome 11.

74. The method of claim 72, wherein the breast cancer gene comprises a locus that maps to human chromosome 11q13-q14.

75. The method of claim 71, comprising providing at least one expression vector comprising a polynucleotide sequence selected from among the polynucleotide sequences of (a), (b), (c), (d), (e) or (f).

76. The method of claim 71, comprising providing at least one probe comprising a polynucleotide sequence selected from among the polynucleotide sequences of (a), (b), (c), (d), (e) or (f); and,
hybridizing the at least one probe to an expression product of a breast cancer gene.

77. The method of claim 71, wherein providing the at least one nucleic acid comprises amplifying a target sequence comprising a polynucleotide sequence selected from among the polynucleotide sequences of (a), (b), (c), (d), (e) or (f).

78. The method of claim 77, wherein the amplifying comprises a quantitative reverse transcriptase-polymerase chain reaction (RT-PCR).

79. The method of claim 77, comprising identifying a target sequence that is differentially expressed in a transformed breast cell compared to a non-transformed breast cell.

80. The method of claim 79, wherein the transformed breast cell comprises a positive estrogen receptor (ER+) cell.

81. The method of claim 71, further comprising detecting altered expression or activity of a product encoded by the at least one nucleic acid comprising a polynucleotide sequence selected from among the polynucleotide sequences of (a), (b), (c), (d), (e) or (f).

82. The method of claim 81, wherein the altered expression or activity of the product is determined by analysis of massively parallel signature sequence data.

83. The method of claim 81, wherein the altered expression or activity is determined to be differentially expressed to a p<0.01 level of confidence.

84. The method of claim 81, wherein the altered expression or activity is determined to be differentially expressed to a p<0.001 level of confidence.

85. The method of claim 81, comprising detecting altered expression in a transformed cell.

86. The method of claim 81, comprising detecting altered expression in response to a carcinogenic signal.

87. The method of claim 81, comprising detecting altered expression in response to tamoxifen.

88. A method of detecting breast cancer in a subject, the method comprising:
(i) providing a subject cell or tissue sample of nucleic acids; and,
(ii) detecting at least one polymorphic nucleic acid or at least one expression product corresponding to a polynucleotide sequence comprising:
(a) at least one polynucleotide sequence selected from the group consisting of: SEQ ID NO: 1-SEQ ID NO: 491 or a polynucleotide sequence complementary thereto;
(b) at least one polynucleotide sequence comprising a conservative variation of a polynucleotide sequence of (a);
(c) at least one polynucleotide encoding a polypeptide sequence of SEQ ID NO: 492, or conservative variations thereof;
(d) at least one polynucleotide sequence that hybridizes under stringent conditions to a polynucleotide sequence of (a) or (b);
(e) at least one polynucleotide that is at least about 70% identical to a polynucleotide sequence of (a), or (b); or,
(f) at least one polynucleotide sequence comprising at least about 10 contiguous nucleotides of a polynucleotide sequence selected from the group consisting of: SEQ ID NO: 1-SEQ ID NO: 491, or a sequence complementary thereto,
wherein the polymorphic nucleic acid or expression or activity of the expression product is correlatable to breast cancer.

89. The method of claim 88, wherein the expression product comprises an RNA.

90. The method of claim 88, wherein the expression product comprises a protein or polypeptide.

91. The method of claim 85, wherein the detecting step comprises qualitative detection.

92. The method of claim 88, wherein the detecting step comprises quantitative detection.
